(19) United States
(12) Patent Application Publication
(10) Pub. No.: US 2010/0095387 A1
(43) Pub. Date: Apr. 15, 2010

(54) METHODS AND REAGENTS FOR SCREENING NEW DRUGS AND FOR TREATING ION PUMP ASSOCIATED DISORDERS AND DISEASES

(76) Inventors: Martin Smith, Irvine, CA (US); Lutz Hilgenberg, San Juan Capistrano, CA (US)

Correspondence Address: KAUTH , POMEROY , PECK & BAILEY ,LLP, 2875 MICHELLE DRIVE, SUITE 110, IRVINE, CA, 92606, US

(21) Appl. No.: 11/995,609

(22) PCT Filed: Jul. 14, 2006
(86) PCT No.: PCT/US2006/027288

§ 371 Date: Sep. 23, 2009

Related U.S. Application Data
(60) Provisional application No. 60/699,939, filed on Jul. 15, 2005.

(51) Int. Cl. G01N 33/53; A01K 67/027; A61K 38/16; A61P 25/16; A61P 9/12

(52) U.S. Cl. 800/3; 435/7.21; 800/18; 514/12

(57) ABSTRACT

The invention relates to fragments of a mammalian nervous system protein, agrin, and to their use as a screening and therapeutic agents in controlling neural activity associated with the function of the Na⁺/K⁺-ATPase pump's function in neurons. Na⁺/K+-ATPases are the most important active transporters in animal cells, required for maintaining the electrochemical gradient responsible for resting membrane potential and function of other transport proteins. Accordingly, the current invention demonstrates agrin's ability to modulate activity of the α3Na⁺/K⁺-ATPase and suggests a direct role in controlling activity-dependent processes in neurons and other excitable cells, including cardiac muscle fibers, providing a molecular framework for identifying treatments for a variety of disorders characterized by dysregulation of cellular excitability such as epilepsy, nervous tissue trauma and coronary diseases.

### STATEMENT REGARDING FEDERALLY SPONSORED RESEARCH OR DEVELOPMENT

This invention was made with Government support under Grant No. NS33213, awarded by the National Institutes of Health. The Government has certain rights in this invention.

### INTRODUCTION

Agrin, a heparan sulfate proteoglycan, was originally isolated from the electric organs of marine rays based on its ability to induce the formation of high density clusters of acetylcholine receptors (AChR) on the surface of cultured muscle cells (Nitkin et al., 1987). It is present at the earliest nerve-muscle contacts during development (Fallon et al., 1985) and, in mature muscles, is localized to the synaptic basal lamina that lies between the axon terminal and muscle fiber (Reist et al., 1987). Agrin is synthesized by motor neurons and antibodies against agrin block formation of motor neuron-induced clusters of AChR on cultured muscle cells (Reist et al., Neuron 8, 865-868, 1992). When expressed in muscle fibers in vivo, agrin induces formation of ectopic postsynaptic structures (Cohen et al., 1997), whereas mutation of the agrin gene blocks accumulation of AChR at developing neuromuscular junctions (Gautam et al., 1996). Thus, agrin is both sufficient and necessary for differentiation of the postsynaptic apparatus of the neuromuscular junction.

For example, in vertebrate skeletal muscle, agrin activation of the receptor tyrosine kinase MuSK mediates motor neuron induced accumulation of acetylcholine receptors at the developing neuromuscular junction (Sanes, J. R. & Lichtman, J. W., (2001)). Agrin is also expressed in brain where it has been implicated in a wide range of neuronal functions including synapse formation, plasticity, process growth, and calcium homeostasis (Smith, M. A. & Hilgenberg, L. Q., (2002)). Indeed, several lines of evidence suggest that agrin is also important for brain development. It is expressed by all populations of neurons in brain (O'Connor et al., 1994) and concentrated at interneuronal synapses (Mann and Kröger, 1996; Hoover et al., 2003). Moreover, the highest levels of agrin expression in developing brain coincide with periods of synapse formation (Cohen et al., 1997; Li et al., 1997). These observations suggest a function analogous to its role at the neuromuscular junction and, consistent with this hypothesis, synapse formation between cultured hippocampal neurons is disrupted when either agrin expression or function is suppressed (Ferreira, 1999; Böse et al., 2000).

However, the molecular identity of the receptor(s) responsible for agrin's diverse effects in neural tissue is not known. For example, the details of agrin signaling and AChR clustering are largely a mystery, but muscle-specific kinase (MuSK) has been identified as the agrin receptor that initiates this process. MuSK alone does not form a functional agrin receptor; rather, it forms a heterodimeric complex with an as yet to be identified myotube-associated specificity component (MASC) to transduce its signal. Agrin has also been shown to bind a number of other cell surface components (i.e., laminin, integrin, tenascin, α-dystroglycan, etc.) and it likely has other functions within the peripheral nervous system (PNS), but binding MuSK-MASC and stewarding synaptogenesis at the neuromuscular junction appears to be its most important role.

Determining agrin's interaction with MuSK (and other effector molecules) was facilitated by detailed structural analysis of agrin (FIG. 2). Agrin is a ˜400-kD heparan sulfate proteoglycan assembled on an ˜200-kD polypeptide backbone. Conceptually, it can be divided into two parts: nine domains homologous to follistatin and one laminin B-like domain in the N-terminal half of agrin, and four EGF-like domains with three laminin A G-like domains in the C-terminal portion. The N-terminus's tertiary structure is globular, and even though consensus sequences for glycosylation exist throughout the protein, the attachment sites for all heparan sulfate glycosaminoglycan side chains lie in the N-terminal half of the protein. The C-terminal portion connects to the N-terminal half via a central rod and has three globular domains important for receptor binding.

AChR clustering activity resides in the C-terminal half of agrin and further structural analysis of the protein revealed a number agrin isoforms with meaningful exon variations near the signaling domain. These isoforms are expressed differentially in the PNS and central nervous system (CNS) and depend upon cell type and developmental stage. Two transcriptional start sites give rise to long and short agrin isoforms and correspond to secreted and membrane bound permutations of the molecule, respectively. Alternative splicing also occurs at three sites within agrin, but the site closest to the C-terminus (the z site) is most important with respect to in vivo agrin function. Analysis of agrin constructs with different exon configurations at the z site not only demonstrated that AChR clustering resides in the third laminin AG-like domain, but also showed AChR clustering is exquisitely sensitive to z+ splice variants. However, CNS cell populations respond to agrin irrespective of exon configuration at the z site, suggesting novel functions and binding partners for agrin in the CNS.

Since MuSK-MASC binding requires an appropriate z+ agrin isoform and MuSK is not expressed in the mammalian brain, it is highly probable that agrin mediates its effects via a unique agrin receptor present only in the CNS. Accumulating evidence also suggests that agrin's role in the CNS is broader and more far-reaching than its known function in the PNS. For example, agrin alters rates of axonal and dendritic elongation and stimulates differentiation of presynaptic terminals. Functional agrin also plays a role in permeate ion homeostasis. In addition, a mechanism that links agrin to the distribution of Na channels has been proposed and agrin deficient neurons demonstrate altered responses to transient changes in cytoplasmic Ca²⁺.

Agrin's involvement in so many fundamental brain processes suggests the potential therapeutic utility of modulating agrin function. Neurofibrillary tangles, senile plaques and amyloid angiopathy characterize the neuropathology of Alzheimer's Disease. Since agrin binds β-amyloid, is a major component of both tangles and plaques, and accelerates fibril formation, its importance to Alzheimer's disease is clear. Agrin's role as a regulator of neuron growth and synaptic plasticity also posits it as a candidate for involvement in traumatic brain injury (TBI) and epilepsy. Axon and dendrite elongation is at least partially regulated by agrin and presents an attractive therapeutic strategy for rescuing neuronal function following TBI. In cultured neurons, agrin influences synaptic efficacy and neural sensitivity to excitatory neurotransmitters. Moreover, heterozygous agrin-deficient mice demonstrate increased resistance to kainic acid-induced seizures. These findings suggest that blockade or control of agrin function following seizures may have therapeutic value in controlling seizure-induced brain injury and/or dampening the kindling phenomena observed seizure disorders. For a review of the role of agrin in the central nervous system, see, Smith, M. A. and Hilgenberg, L. G. W. (2002), herein incorporated by reference in its entirety.

Recently, using small fragments of agrin as probes, it has been demonstrated that receptors for agrin are concentrated at synapses formed between central nervous system neurons (Hoover, C. L., et. al., (2003)). In the current invention it is shown that this agrin receptor is the α3 subunit of the Na⁺/K⁺-ATPase and that agrin specifically inhibits this pump's function in neurons. Na⁺/K⁺-ATPases are the most important active transporters in animal cells, required for maintaining the electrochemical gradient responsible for resting membrane potential and function of other transport proteins (Kaplan, J. H., (2002)). Accordingly, agrin's ability to modulate activity of the α3Na⁺/K⁺-ATPase suggests a direct role in controlling activity-dependent processes in neurons and provides a molecular framework for agrin function in the central nervous system that leads to a number of different tests and treatments for disorders and diseases related to the function of the Na⁺/K⁺-ATPase pump.

### SUMMARY OF THE INVENTION

The present invention is directed to methods of using fragments of agrin and an agrin receptor, namely the α3 subunit of the Na⁺/K⁺-ATPase, in screening for treatments for and treating ion pump associated disorders.

Thus, in one embodiment the invention is directed to a method for identifying cardiac glycosides or other small molecules useful for treating congestive heart failure that potentially have reduced or no neurological side effects. In one such embodiment the method includes preparing derivatives of a cardiac glycoside that bind to either or both of the α1Na⁺/K⁺-ATPase and α2Na⁺/K⁺-ATPase receptors, but that do not bind to α3Na⁺/K⁺-ATPase receptor.

In another embodiment the invention is directed to a method for screening therapeutic agents useful for treating seizures. In one such embodiment the method includes contacting a potential therapeutic agent to an α3Na⁺/K⁺-ATPase receptor and evaluating the ability of the potential therapeutic agent to potentiate α3Na⁺/K⁺-ATPase activity.

In still another embodiment the invention is directed to a method for screening therapeutic agents potentially useful for treating congestive heart disease. In one such embodiment the method includes identifying those potential therapeutic agents that inhibit α1Na⁺/K⁺-ATPase and/or α2Na⁺/K⁺-ATPase activity but not α3Na⁺/K⁺-ATPase activities.

In yet another embodiment the invention is directed to a method for screening therapeutic agents potentially useful for treating hypertension. In one such embodiment the method includes identifying those potential therapeutic agents that inhibit α1Na⁺/K⁺-ATPase and/or α2Na⁺/K⁺-ATPase activity but not α3Na⁺/K⁺-ATPase activities.

In still yet another embodiment the invention is directed to an ATP1a3^{loxP}/^{loxP} Th^{cre}/^{cre} transgenic mouse in which expression of Cre recombinase results in loss of α3Na⁺/K⁺-ATPase function in dopamine neurons, resulting in abnormal motor performance.

In still yet another embodiment the invention is directed to a method of screening biologically active agents that facilitate improvement in motor performance. In one such embodiment the method includes administering a candidate agent to a transgenic mouse and determining the effect of the agent upon motor performance.

### BRIEF DESCRIPTION OF THE DRAWINGS

These and other features and advantages of the present invention will be better understood by reference to the following detailed description when considered in conjunction with the accompanying drawings.

FIG. 1. Model of agrin function at the synapse. The resting Na⁺/K⁺ electrochemical gradient depends on activity of local α3Na⁺/K⁺-ATPases; some α3Na⁺/K⁺-ATPases are bound to and inhibited by agrin at rest. Further inhibition of α3Na⁺/K⁺-ATPases by agrin results in collapse of the Na⁺ gradient within a diffusion-restricted physiological space leading to slowing or even reversal of the NCX and a rapid rise in cytoplasmic Ca2+ concentration. Depolarization of synaptic membranes associated with the increase in intracellular Na+ concentration following α3Na+/K+-ATPase inhibition also triggers Ca2+ influx through voltage-gated channels. Increased cytoplasmic Ca2+, augmented by Ca2+ induced Ca2+ release from intracellular stores via ryanodine receptors (Hilgenberg and Smith, 2004), activates CaMKII and other Ca2+ effectors known to regulate a variety of synaptic functions such as neurotransmitter release and neurotransmitter receptor turnover. Agrin and the α3Na+/K+-ATPase are shown on both pre- and postsynaptic membranes, however their precise subcellular location remains to be determined.

FIG. 2 is a schematic diagram showing the structure of agrin and agrin deletion constructs. Alternate transcriptional start sites give rise to short and long NH2-terminal (SN, LN) forms of agrin. Agrin's polypeptide chain is characterized by numerous cysteine-rich repeats similar to follistatin (F), laminin B (LB), EGF (E), and laminin A G domains (G). Two serine/threonine-rich regions (S/T), consensus glycosaminoglycans side-chain attachment sites (lollipops), and sites of alternative splicing (X, Y, Z) are also shown. Horizontal bars indicate location of binding sites for various cell surface and ECM molecules. The minimal region required for agrin's AChR clustering activity is also shown. Agrin deletion constructs C-Ag95_{z0/8} and C-AgΔ20 included an NH₂-terminal signal peptide for expression in mammalian cells and 4 amino acid insert at the y site. All deletion constructs included COOH-terminal myc (m) and polyhistidine (H) epitope tags.

FIG. 3 shows induction of expression of c-fos in cultured cortical neurons by C-Ag95_{z8} and C-Ag95_{z0}. (A) 12-d-old cortical cultures were treated for 10 min with either C-Ag95_{z8} or C-Ag95_{z0}, followed by double labeling with antibodies for Fos (fluorescein channel) and either MAP2 or GFAP (rhodamine channel). Cell bodies and nuclei of MAP2-positive neurons were intensely labeled for Fos in cultures treated with either C-Ag95_{z8} or C-Ag95_{z0}. In contrast, only basal levels of Fos expression were observed in GFAP-positive nonneuronal cells. Induction of c-fos was agrin specific in that no detectable increase in Fos was apparent in cultures treated with prostate serum antigen control protein. Bar, 20 μm. (B) Cultures were incubated for 10 min in C-Ag95_{z8} (open circles, broken line) or C-Ag95_{z0} (filled circles, solid line), and levels of Fos expression were determined by in situ enzyme-linked assay as described herein. Both agrin constructs induced essentially identical concentration dependent and saturable increases in c-fos expression. Data were normalized to maximal level of Fos expression in each experiment. Each point represents the mean of triplicate determinations from three independent experiments±SEM. Curves were fit by single-site nonlinear regression model R²≧0.94. Values from mock-treated sister wells have been subtracted.

FIG. 4 is a graph showing that the 20-kD COOH-terminal region of agrin is necessary and sufficient for induction of c-fos. (A) Cortical neurons were treated for 10 min with C-AgΔ20 alone or in the presence of 50 pM C-Ag95_{z8} or C-Ag95_{z0}. Neither a subsaturating (50 pM) nor supersaturating (50 nM) concentration of C-AgΔ20 induced expression of c-fos, nor were they able to modulate the activity of the larger fragments. (B) In contrast, cortical cultures exhibited a concentration-dependent and saturable increase in response to C-Ag20_{z8} (open circles, broken line) or C-Ag20_{z0} (solid circles, solid line) similar to that seen with the C-Ag95_{z8/0} fragments. Data were normalized to the level of Fos expression induced by 50 pM C-Ag95_{z8} alone in A and maximal level of Fos expression in B. Each data point shows the mean of triplicate determinations from three independent experiments±SEM. Curves were fit by single-site nonlinear regression model R²≧0.97. Background levels of Fos expression from mock-treated sister cultures have been subtracted.

FIG. 5 is a graph showing that the 15-kD COOH-terminal region of agrin is a cell-specific competitive inhibitor of C-Ag95_{z8} induction of c-fos. (A) Cortical cultures were incubated in 50 pM C-Ag95_{z8} in the presence of different concentrations of C-Ag15. C-Ag15 inhibited C-Ag95_{z8} induction of c-fos in a concentration-dependent manner well described by a single-site competition model (R²=0.95) with an IC₅₀ of 64.5 pM; close to a 1:1 agonist:antagonist molar ratio. Data were normalized to percentage of Fos expression induced by 50 pM C-Ag95_{z8} alone, and represent the mean of triplicate determinations from three independent experiments±SEM. Background levels of Fos expression have been subtracted. (B) To test the cell specificity of C-Ag15 inhibition, hippocampal (Hip) or cerebellar (Cer) neurons and chick muscle fibers (Mus) were incubated in 50 pM C-Ag95_{z8} alone (open bars) or in the presence (filled bars) of 1 nM C-Ag15. Levels of Fos expression were expressed as fold change over mocktreated sister cultures such that a value of 1 indicates no change. C-Ag15 completely inhibited C-Ag95_{z8}-induced expression of Fos in hippocampal and cerebellar neurons, but not in muscle. (C) The effect of C-Ag15 on C-Ag95_{z8}-induced AChR aggregation was also tested. Cultured chick myotubes were incubated overnight in 50 pM C-Ag95_{z8} alone (−) or in the presence (+) of 1 nM C-Ag15, and AChR clusters were labeled with rhodamine-conjugated α-bungarotoxin. AChR clusters were counted blind with respect to treatment in five random fields/well and expressed as the ratio of clusters in mock-treated sister cultures. Consistent with the results of the Fos expression analysis, C-Ag15 had no effect on C-Ag95_{z8}-induced AChR clustering. Bars in B and C represent the mean±SEM of triplicate wells from four independent experiments.

FIG. 6 is a graph showing that the 20-kD COOH-terminal region of agrin is the minimal fragment sufficient to rescue an agrin-deficient phenotype. (A) 10-d-old wild-type (filled bars) or agrin-deficient (open bars) cultures were grown for 2 d in media supplemented to 5 nM with the indicated agrin fragment. Cultures were challenged for 5 min with 100 μM glutamate as described previously (Hilgenberg et al., 2002), and the levels of Fos expression were determined. Data were normalized to levels of glutamate-induced Fos expression in mocktreated wild-type cultures. Compared with mock, glutamate responses of homozygous agrin-deficient neurons were rescued to near wildtype levels by either the long C-Ag95_{z8/0} or short C-Ag20_{z8/0} fragments (***, P≦0.0005; **, P≦0.002; *, P≦0.02; paired t test). (B) To test the ability of C-Ag15 to antagonize agrin action, 10-d-old agrin-deficient neurons were maintained for 2 d in the presence of 5 nM C-Ag95_{z0} alone or in combination with 10 nM C-Ag15, and neuronal responses to glutamate were determined as above. Treatment with C-Ag15 significantly (*, P≦0.004; paired t test) inhibited rescue of the agrin-deficient response to glutamate by C-Ag95_{z0}.

FIG. 7 is a micromicrophotograph showing agrin receptor expression in nerve cell membranes. Live cortical neurons were incubated in C-Ag20_{z8}, C-Ag20_{z0}, or C-Ag15, either alone or in the presence of mock-conditioned medium or a 500-fold molar excess of the active (Rat C-Ag_{4,8}) or inactive (Rat C-Ag_{0,0}) isoform of rat agrin. Immunostaining with an anti-polyhistidine antibody reveals binding of the short agrin fragments to receptors distributed in numerous small clusters on neuron cell bodies and neurites; patches of agrin receptors outside the focal plane contribute to the diffuse staining evident in some neuron cell bodies. Consistent with a single class of agrin receptors, each fragment shows a similar pattern of binding that can be blocked by either isoform of rat agrin. The ability of rC-Ag to block the short agrin fragments is also strong evidence that binding is specific. In addition, no labeling was observed when the short agrin fragments were omitted or replaced by β-galactosidase (β-Gal) as a control for vector-specific sequences. Bar, 20 μm.

FIG. 8 is a photomicrograph showing agrin and agrin receptors colocalized at synaptic contacts. The subcellular distribution of endogenous agrin and agrin receptors on cultured cortical neurons was determined by labeling with either an anti-agrin serum, RαAg-1, or short agrin fragment, followed by fixation and incubation with an antibody to synaptophysin to identify nerve terminals by labeling synaptic vesicles. Both agrin and the agrin receptor were present at virtually all synaptophysin-positive nerve terminals (arrowheads), evidence that agrin and its receptor are colocalized at synaptic sites. Nerve terminal staining was specific, and was not observed in control cultures labeled with an agrin receptor probe in the absence of the anti-synaptophysin antibody. Bar, 20 μm.

FIG. 9. Agrin binds to and induces tyrosine phosphorylation of a ˜110 kDa protein on neuron surface membranes. a. Immunoblots probed with an antibody to the myc tag on the agrin fragments show agrin adducts from neurons but not non-neuronal cells cross-linked to C-Ag20₈ or C-Ag15. b. Agrin adducts were immunoprecipitated with either an agrin antiserum (Agrin) or anti-phosphotyrosine antibody (PY) and then analyzed by immunoblotting for the myc tag. Cross-linking to C-Ag20₈ or C-Ag15 alone results in the appearance of the appropriately sized bands, but only the 125 kDa band was present when C-Ag20₈ was cross-linked in the presence of C-Ag15. Phosphorylation of the cross-linked complex is induced by C-Ag20₈ but not C-Ag15. Consistent with the competition studies, C-Ag20₈-induced phosphorylation of the agrin adduct is blocked by C-Ag15.

FIG. 10. Agrin binds specifically to the α3Na⁺/K⁺-ATPase on central nervous system neurons. a. Immunoblots of cultured cortical neurons cross-linked to different agrin fragments were probed with monoclonal antibodies against either the α3- or α1Na⁺/K⁺-ATPase. Consistent with the results of the mass spectrometry, only the α3Na⁺/K⁺-ATPase shows the expected increases in molecular weight. b. Cortical neurons were double labelled for α3Na⁺/K⁺-ATPase and C-Ag20₈ binding sites. Consistent with the biochemical analysis, agrin binding sites and α3Na⁺/K⁺-ATPase are colocalized, appearing as small puncta distributed over the surface of the neuron soma and neurites. c. Cortical neurons were double labeled for α3Na⁺/K⁺-ATPase and synaptophysin binding sites. Consistent with the biochemical studies, agrin binding sites show agrin receptors diffusely distributed over the neuronal soma but concentrated at synapses.

FIG. 11. Agrin inhibits α3Na+/K+-ATPase function. a. Pseudocolor images of cells loaded with the Na+ sensitive dye SBFI-AM, before (Control) and 90 s after exposure to C-Ag208. Na+ levels increase in the neurons (arrows) but not in non-neuronal cells (arrowheads) following agrin treatment. Scale bar=20 μm. b. Treatment with C-Ag208 triggers a rapid increase in neuronal intracellular Na+ (solid line, arrow in a) that returns to initial resting level upon being washed into normal saline solution (S). The small response in the non-neuronal cell (broken line, arrowhead in a) is due to fine neurites traversing the sampled region. c. Neuronal Na⁺ levels are unchanged following treatment with C-Ag15 alone, but C-Ag15 blocks the large increase induced by C-Ag200. d. Treatment with a saturating concentration of either C-Ag20 isoform or C-Ag908 all resulted in a significant increase in intracellular Na+ concentration, expressed as a percent of the maximal response to gramicidin, that could be blocked by co-incubation with CAg15. e. The increase in neuronal Na+ levels induced by 3 μM ouabain was significantly reduced by co-incubation with C-Ag15. f. Whole cell current clamp record showing reversible membrane depolarization produced in a neuron by treatment with C-Ag208. g. Treatment with C-Ag15 resulted in a small hyperpolarization and blocked the change in membrane potential induced by C-Ag200. h. Treatment with “active” fragments of agrin causes membrane depolarization whereas the membrane potentials of cells exposed to C-Ag200 or C-Ag208 in the presence of C-Ag15 were indistinguishable from their normal resting membrane potentials obtained before treatment (data for C-Ag200 and C-Ag208 were not different and have been pooled). i. Ouabain induced neuron membrane depolarization is also blocked by C-Ag15. Bars show mean±SEM. *p<0.05, **p<0.01, ***p<0.001; paired Student's t-test.

FIG. 12. Expression of the α3 subunit of the Na+/K+-ATPase in non-neuronal cells confers binding and functional response to agrin. a. Transfected and non-transfected cells were visualized by post-labeling for GFAP. Only EGFP positive cells co-transfected with pEGFP-C1 and pRca3 bind agrin (C-Ag200). Asterisks indicate non-transfected cells. Scale bar=10 μm. b. An immunoblot probed with an antibody to the α3Na+/K+-ATPase. The α3 subunit is expressed in transfected (T) but not sham transfected (S) control cells and can be cross-linked to agrin. c. The DIC image shows a pair of cells in which only the lower cell has been transfected with pRca3, indicated by expression of EGFP. The pseudocolor images show that, compared to saline, treatment with C-Ag200 results in a marked increase in intracellular Na+ concentration in the transfected but not the non-transfected cell. Scale bar=10 μm. d. Bar chart shows mean response to different agrin fragments of non-neuronal cells transfected with either pEGFP-C1 alone (filled bar) or in combination with pRcα3. Responses of pEGFP-C1 transfected cells to C-Ag200 and C-Ag208 have been pooled. ***p<0.001; ANOVA with Bonferroni post-hoc comparison to pEGFP-C1 control. e. pRcα3 expression in non-neuronal cells confers an agrin-inducible reversible membrane depolarization that can be blocked by C-Ag15. f. Nonneuronal cells expressing the α3 subunit of the NA+/K+-ATPASE but not control (non-transfected or pEGFP-C1 transfected) cells are consistently depolarized (***p<0.001; paired Student's t-test) by treatment with agrin.

FIG. 13. Frequency of spontaneous action potentials in cultured neurons is agrin dependent. a. A typical record showing the reversible membrane depolarization and increased frequency of spontaneous action potentials in a neuron treated with C-Ag200. b. Increase in mean frequency of spontaneous action potentials of individual neurons in normal saline followed by C-Ag200 (p<0.02, Wilcoxon signed rank test). c. Bath application of C-Ag15 results in a reversible decrease in the frequency of spontaneous action potentials. Slight hyperpolarization of the resting membrane potential is also apparent. d. The mean frequency of action potentials in individual neurons is consistently reduced by treatment with C-Ag15 (p<0.001, Wilcoxon signed rank test).

FIG. 14. Neuronal activity in vivo is regulated by endogenous agrin-α3Na+/K+-ATPase interactions. a. Detergent solubalized membranes of BS3 cross-linked cultured cortical neurons or cortical slice were immunoprecipitated (IP) with antibodies against either agrin (Ag) or the α3Na+/K+-ATPase (α3) and then immunoblotted for α3Na+/K+-ATPase. BS3 cross-linking of either cultured neurons or cortex results in the formation of ±300 kDa adduct, indicative of the presence of native agrin-α3Na+/K+-ATPase complexes. Endogenous agrin-α3Na+/K+-ATPase interactions can be disrupted by incubation with CAg15, resulting in the appearance of 125 kDa C-Ag15-α3Na+/K+-ATPase band. For comparison, the 110 kDa α3Na+/K+-ATPase visualized by immunoprecipitation and immunoblotting with the same α3Na+/K+-ATPase antibody in the absence of cross-linking is shown. b. A whole cell current clamp record from layer V neuron in a slice of motor cortex. Treatment with agrin results in reversible membrane depolarization and appearance of high frequency action potentials. c. The mean action potential frequency of individual neurons was significantly increased by treatment with C-Ag200 (p<0.01, Wilcoxon signed rank test). d. A typical current clamp record showing action potentials induced by treatment with kainic acid (K). Addition of C-Ag15 results in reversible membrane repolarization and blockade of action potentials. e. C-Ag15 consistently inhibited firing of kainite-induced action potentials in individual neurons (p<0.01, Wilcoxon signed rank test).

FIG. 15. C-Ag15 is neuroprotective for excitotoxic injury. Cultured cortical eurons were treated for 10 minutes with the excitatory neurotransmitter glutamate in the presence of the indicated concentration of C-Ag15 and degree of neuronal injury assessed by monitoring the level of LDH in the growth medium. Data are expressed as a percentage of the LDH in sister cultures treated with glutamate alone. The neuroprotective effect of C-Ag15 is dose-dependent.

FIG. 16. Model of agrin signaling in a cardiac myocyte. The Na+/K+ ion gradient in cardiac myocytes is dependent on the activity of α1-, α2- and α3 subunit containing Na+/K+-ATPAse isoforms. By analogy to agrin function in CNS neurons (see Hilgenberg et al., 2006), agrin binding causes phosphorylation (red ball) and inhibition of the α3Na+/K+-ATPase. Collapse of the Na+ gradient in the diffusion restricted space between the sarcolemma and SR leads to slowing or reversal of NCX1 and rise in cytoplasmic Ca2+. Depolarization of the sarcolemma stimulates Ca2+ influx through voltage-gated L-type channels, augmented by ryanodine receptor mediated Ca2+-induced Ca2+ release from the SR. Even at rest, some α3Na+/K+-ATPase molecules are bound by agrin. Agrin is shown attached to the basal lamina and the α3Na+/K+-ATPase concentrated in and around the T-tubule, however, the subcellular location of these molecules remains to be determined. In this figure α1-, and α2Na+/K+-ATPase have been omitted for clarity.

FIG. 17. Cardiac myocyte contraction is agrin dependent. Cardiac myocytes were prepared from hearts of individual embryos produced by heterozygous pairings. The genotype of cultures was determined by PCR analysis of tissue samples from each embryo. Myocytes were maintained in 199 medium containing 10% FCS as described (Mitcheson et al., 1998). At 5 days in culture, the frequency of spontaneous contractions was determined by counting contractions in myocytes from 5 random fields at 300× magnification for 30 seconds. Contraction frequency is dependent on Agrn gene dosage such that Agrn−/−>Agrn+/−>Agrn+/+. Addition of C-Ag208 to the growth medium rescues the Agrn mutant phenotype whereas C-Ag15 increased the contraction frequency of wild type myocytes to a level similar to Agrn−/− cells. Bars show mean±SEM for a minimum of 2 independent platings from 3 embryos, except for Agrn+/−treated with C-Ag208, which was a single plating of 2 embryos. All data were collected blind with respect to genotype and agrin treatment. *** p≦0.001. ANOVA with Bonferroni post-hoc pair-wise comparison.

FIG. 18. Agrin and the α3Na+/K+-ATPase are co-localized in cardiac myocytes. Four-day-old cultured myocytes, prepared from 18-day-old mouse embryos, were fixed and double labeled with a rabbit anti-agrin antibody and either an anti-α3Na+/K+-ATPase or anti-α1Na+/K+-ATPase mouse monoclonal. Agrin and the α3Na+/K+-ATPase appear to be co-localized in fine puncta broadly distributed over the cell surface. In contrast, the α1Na+/K+-ATPase is concentrated along regions of contact between adjacent cells. No labeling was evident in control cultures in which the primary antibody was omitted (not shown). Scale bar=20 μM.

FIG. 19. Agrin regulates cytoplasmic Na+ and Ca2+ ion concentrations in cardiac myocytes. (A) DIC image shows confluent field of cardiac myocytes at 4 days in culture loaded with Fura-2. Pseudocolor images shows Ca2+ levels in the same cells bathed in saline and 30 s after treatment with C-Ag208. Cells were then returned to normal saline before being treated with 100 μM ouabain to inhibit all Na+/K+-ATPase isoforms. (B) Cytoplasmic Ca2+ and Na+ concentrations are significantly increased in the presence of the active agrin fragment C-Ag208 but not C-Ag15. However, preincubation with a 10 fold excess of C-Ag15 effectively blocks inhibition of the α3Na+/K+-ATPase by C-Ag208. Data are expressed as percent of the maximal response to ouabain. Bar chart summarize results from a minimum of 10 cells from 2 platings.

FIG. 20. Response to agrin is unaffected by mutations that reduce sensitivity to ouabain. When co-transfected with plasmids expressing GFP and ouabain-resistant a3 subunit for the Na+/K+-ATPase, non-neuronal cerebro-cortical cells, which are normally unresponsive to agrin (Hilgenberg et al., 2006) become agrin sensitive. (A) Photomicrographs show, GFP positive non-neuronal cell and pseudocolor images of cytoplasmic Ca2+ concentration in saline and 30 s after treatment with C-Ag208. Chart show time course of the response in the same cell.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

### Publications

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention. Such publications include the following:
Almquist, R. G. et al., J Med Chem (1980) 23:1392-1398.
Altschcul, S. F. et al., J. Mol. Biol. 215:403-410 (1990).
Annunziato, L., Pignataro, G., and Di Renzo, G. F. (2004). Pharmacology of brain Na+/Ca2+ exchanger: from molecular biology to therapeutic perspectives. Pharmacol Rev 56, 633-654.
Ausubel et al., CURRENT PROTOCOLS IN MOLECULAR BIOLOGY (John Wiley & Sons, Inc. 1994-1998).
Avery's Drug Treatment: Principles and Practice of Clinical Pharmacology and Therapeutics, 3rd edition, ADIS Press, LTD.
Barlet-Bas, C., Arystarkhova, E., Cheval, L., Marsy, S., Sweadner, K., Modyanov, N., and Doucet, A. (1993). Are there several isoforms of Na,K-ATPase alpha subunit in the rabbit kidney? J Biol Chem 268, 11512-11515.
Berkow et al., eds., The Merck Manual, 16th edition, Merck and Co., Rahway, N.J. (1992).
Blaustein, M. P., Juhaszova, M., Golovina, V. A., Church, P. J., and Stanley, E. F. (2002) Na/Ca exchanger and PMCA localization in neurons and astrocytes: functional implications. Ann N Y Acad Sci 976, 356-366.
Blaustein, M. P., Zhang, J., Chen, L., and Hamilton, B. P. (2006). How does salt retention raise blood pressure? Am J Physiol Regul Integr Comp Physiol 290, R514-523.
Bodey B., et al., Curr. Pharm. Des. 6:261-76 (2000).
Böse, C. M., Qiu, D., Bergamaschi, A., Gravante, B., Bossi, M., VIIIa, A., Rupp, F., and Malgaroli, A. (2000). Agrin controls synaptic differentiation in hippocampal neurons. J Neurosci 20, 9086-9095.
Bouron, A., and Reuter, H. (1996). A role of intracellular Na+ in the regulation of synaptic transmission and turnover of the vesicular pool in cultured hippocampal cells. Neuron 17, 969-978.
Brines, M. L., Dare, A. O., and de Lanerolle, N. C. (1995). The cardiac glycoside ouabain potentiates excitotoxic injury of adult neurons in rat hippocampus. Neurosci Lett 191, 145-148.
Brines, M. L., and Robbins, R. J. (1993). Cell-type specific expression of Na+, K+-ATPase catalytic subunits in cultured neurons and glia: evidence for polarized distribution in neurons. Brain Res 631, 1-11.
Burgess, R. W., W. C. Skarnes, and J. R. Sanes. 2000. Agrin Isoforms With Distinct Amino Termini Differential Expression, Localization, And Function. J. Cell Biol. 151:41-52.
Burgess, R. W., D. K. Dickman, L. Nunez, D. J. Glass, and J. R. Sanes. 2002. Mapping Sites Responsible For Interactions Of Agrin With Neurons. J. Neurochem. 83:271-284.
Campagna, J. A., Ruegg, M. A., and Bixby, J. L. (1997). Evidence that agrin directly influences presynaptic differentiation at neuromuscular junctions in vitro. Eur J Neurosci 9, 2269-2283.
Campagna, J. A., M. Ruegg, and J. L. Bixby. 1995. Agrin Is A Differentiation-Inducing “Stop Signal” For Motorneurons In Vitro. Neuron. 15:1365-1374.
Campanelli, J. T., S. L. Roberds, K. P. Campbell, and R. H. Scheller. 1994. A Role For Dystrophin-Associated Glycoproteins And Utrophin In Agrin-Induced AChR Clustering. Cell. 77:663-674.
Chen, J. Q., Contreras, R. G., Wang, R., Fernandez, S. V., Shoshani, L., Russo, I. H., Cereijido, M., and Russo, J. (2005). Sodium/potassium ATPase (Na(+), K(+)-ATPase) and ouabain/related cardiac glycosides: a new paradigm for development of anti-breast cancer drugs? Breast Cancer Res Treat, 1-15.
Cohen, I., Rimer, M., Lomo, T., and McMahan, U. J. (1997a). Agrin-induced postsynaptic-like apparatus in skeletal muscle fibers in vivo. Mol Cell Neurosci 9, 237-253.
Cohen, N. A., Kaufmann, W. E., Worley, P. F., and Rupp, F. (1997b). Expression of agrin in the developing and adult rat brain. Neuroscience 76, 581-596.
Coligan et al., CURRENT PROTOCOLS IN PROTEIN SCIENCE (John Wiley & Sons, Inc. 1995-1997).
Cote, P. D., Moukhles, H., Lindenbaum, M., and Carbonetto, S. (1999). Chimaeric mice deficient in dystroglycans develop muscular dystrophy and have disrupted myoneural synapses. Nat Genet. 23, 338-342.
Davidson, D. L., Tsukada, Y., and Barbeau, A. (1978). Ouabain induced seizures: site of production and response to anticonvulsants. Can J Neurol Sci 5, 405-411.
de Carvalho Aguiar, P., Sweadner, K. J., Penniston, J. T., Zaremba, J., Liu, L., Caton, M., Linazasoro, G., Borg, M., Tijssen, M. A., Bressman, S. B., et al. (2004). Mutations in the Na+/K+-ATPase alpha3 gene ATP1A3 are associated with rapid-onset dystonia Parkinsonism. Neuron 43, 169-175.
DeChiara, T. M., Bowen, D. C., Valenzuela, D. M., Simmons, M. V., Poueymirou, W. T., Thomas, S., Kinetz, E., Compton, D. L., Rojas, E., Park, J. S., et al. (1996). The receptor tyrosine kinase MuSK is required for neuromuscular junction formation in vivo. Cell 85, 501-512.
Delamere, N. A., and Tamiya, S. (2004). Expression, regulation and function of Na,K-ATPase in the lens. Prog Retin Eye Res 23, 593-615.
Denzer, A. J., M. Gesemann, B. Schumacher, and M. A. Ruegg. 1995. An Aminoterminal Extension Is Required For The Secretion Of Chick Agrin And Its Binding To Extracellular Matrix. J. Cell Biol. 131:1547-1560.
Denzer, A. J., Brandenberger, R., Gesemann, M., Chiquet, M., and Ruegg, M. A. (1997). Agrin binds to the nerve-muscle basal lamina via laminin. J Cell Biol 137, 671-683.
Dostanic, I., Paul, R. J., Lorenz, J. N., Theriault, S., Van Huysse, J. W., and Lingrel, J. B. (2005). The alpha2-isoform of Na—K-ATPase mediates ouabain-induced hypertension in mice and increased vascular contractility in vitro. Am J Physiol Heart Circ Physiol 288, H477-485.
Ebadi, Pharmacology, Little, Brown and Co., Boston (1985).
Evans et al. (1987) J. Med. Chem. 30: 1229.
Fallon, J. R., Nitkin, R. M., Reist, N. E, Wallace, B. G., and McMahan, U. J. (1985). Acetylcholine receptor-aggregating factor is similar to molecules concentrated at neuromuscular junctions. Nature 315, 571-574.
Fauchere, J. (1986) Adv. Drug Res. 15: 29.
Ferns, M., W. Hoch, J. T. Campanelli, F. Rupp, Z. W. Hall, and R. H. Scheller. 1992. RNA Splicing Regulates Agrin-Mediated Acetylcholine Receptor Clustering Activity On Cultured Myotubes. Neuron. 8:1079-1086.
Ferns, M. J., J. T. Campanelli, W. Hoch, R. H. Scheller, and Z. Hall. 1993. The Ability Of Agrin To Cluster Achrs Depends On Alternative Splicing And On Cell Surface Proteoglycans. Neuron. 11:491-502.
Ferraiolo, B. L., and Pace, D. G. (1979). Digoxin-induced decrease in intraocular pressure in the cat. Eur J Pharmacol 55, 19-22.
Ferreira, A. (1999). Abnormal synapse formation in agrin-depleted hippocampal neurons. J Cell Sci 112, 4729-4738.
Gautam, M., Noakes, P. G., Moscoso, L., Rupp, F., Scheller, R. H., Merlie, J. P., and Sanes, J. R. (1996). Defective neuromuscular synaptogenesis in agrin-deficient mutant mice. Cell 85, 525-536.
Gee, S. H., F. Montanaro, M. H. Lindenbaum, and S. Carbonetto. 1994. Dystroglycan-α, A Dystrophin-Associated Glycoprotein, Is A Functional Agrin Receptor. Cell. 77:675-686.
Gelman, D. M., et al., Genesis 36: 196-202 (2003).
Gesemann, M., A. J. Denzer, and M. A. Ruegg. 1995. Acetylcholine Receptor-Aggregating Activity Of Agrin Isoforms And Mapping Of The Active Site. J. Cell Biol. 128:625-636.
Gesemann, M., V. Cavalli, A. J. Denzer, A. Brancaccio, B. Schumacher, and M. A. Ruegg. 1996. Alternative Splicing Of Agrin Alters Its Binding To Heparin, Dystroglycan, And The Putative Agrin Receptor. Neuron. 16:755-767.
Ghosh, S., Freitag, A. C., Martin-Vasallo, P., and Coca-Prados, M. (1990). Cellular distribution and differential gene expression of the three alpha subunit isoforms of the Na,K-ATPase in the ocular ciliary epithelium. J Biol Chem 265, 2935-2940.
Ghosh, S., Hernando, N., Martin-Alonso, J. M., Martin-Vasallo, P., and Coca-Prados, M. (1991). Expression of multiple Na+,K(+)-ATPase genes reveals a gradient of isoforms along the nonpigmented ciliary epithelium: functional implications in aqueous humor secretion. J Cell Physiol 149, 184-194.
Gibbs, M. E., and Ng, K. T. (1978). Memory formation for an appetitive visual discrimination task in young chicks. Pharmacol Biochem Behav 8, 271-276.
Gingras, J., and M. Ferns. 2001. Expression And Localization Of Agrin During Sympathetic Synapse Formation In Vitro. J. Neurobiol. 48:228-242.
Gingras, J., S. Rassadi, E. Cooper, and M. Ferns. 2002. Agrin Plays An Organizing Role In The Formation Of Sympathetic Synapses. J. Cell Biol. 158:1109-1118.
Glass, D. J., Bowen, D. C., Stitt, T. N., Radziejewski, C., Bruno, J., Ryan, T. E., Gies, D. R., Shah, S., Mattson, K., Burden, S. J., et al. (1996). Agrin acts via a MuSK receptor complex. Cell 85, 513-524.
Glitsch, H. G. (2001). Electrophysiology of the sodium-potassium-ATPase in cardiac cells. Physiol Rev 81, 1791-1826.
Godfrey, E. W., Dietz, M. E., Morstad, A. L., Wallskog, P. A., and Yorde, D. E. (1988). Acetylcholine receptor-aggregating proteins are associated with the extracellular matrix of many tissues in Torpedo. J Cell Biol 106, 1263-1272.
Goodman et al., eds., Goodman and Gilman's The Pharmacological Basis of Therapeutics, 8th edition, Pergamon Press, Inc., Elmsford, N.Y. (1990).
Halloran P. F., et al., Clin. Biochem. 31:353-7 (1998).
Hann, M. M., J Chem Soc Perkin Trans I (1982) 307-314.
Heathcote, R. D., J. M. Ekman, K. P. Campbell, and E. W. Godfrey. 2000. Dystroglycan Overexpression In Vivo Alters Acetylcholine Receptor Aggregation At The Neuromuscular Junction. Dev. Biol. 227:595-605.
Hentikoff and Hentikoff, Proc. Natl. Acad. Sci. USA. 89:10915-10919 (1992).
Herbst, R., and Burden, S. J. (2000). The juxtamembrane region of MuSK has a critical role in agrin-mediated signaling. EMBO J 19, 67-77.
Hilgenberg, L. G., and Smith, M. A. (2004). Agrin signaling in cortical neurons is mediated by a tyrosine kinase-dependent increase in intracellular Ca2+ that engages both CaMKII and MAPK signal pathways. J Neurobiol 61, 289-300.
Hilgenberg, L. G. W., Ho, K. D., Lee, D., O'Dowd, D., and Smith, M. A. (2002). Agrin regulates neuronal responses to excitatory neurotransmitters in vitro and in vivo. Mol Cell Neurosci 19, 97-110.
Hilgenberg, L. G. W., Hoover, C. L., and Smith, M. A. (1999). Evidence of an agrin receptor in cortical neurons. J Neurosci 19, 7384-7393.
Hoch, W. 1999. Formation Of The Neuromuscular Junction. Agrin And Its Unusual Receptors. Eur. J. Biochem. 265:1-10.
Hoch, W., J. T. Campanelli, S. Harrison, and R. H. Scheller. 1994. Structural Domains Of Agrin Required For Clustering Of Nicotinic Acetylcholine Receptors. EMBO J. 13:2814-2821.
Hoch, W., Ferns, M., Campanelli, J. T., Hall, Z. W., and Scheller, R. H. (1993). Developmental regulation of highly active alternatively spliced forms of agrin. Neuron 11, 479-490.
Holladay, M. W. et al., Tetrahedron Lett (1983) 24:4401-4404.
Hoover, C. L., Hilgenberg, L. G. W., and Smith, M. A. (2003). The COOH-terminal domain of agrin signals via a synaptic receptor in CNS neurons. J Cell Biol 161, 923-932.
Hopf, C., and W. Hoch. 1996. Agrin Binding to α-dystroglycan. J. Biol. Chem. 271:5231-5236.
Hruby, V. J., Life Sci (1982) 31:189-199.
Hudson, D. et al., Int J Pept Prot Res (1979) 14:177-185.
Jacobson, C., F. Montanaro, M. Lindenbaum, S. Carbonetto, and M. Ferns. 1998. α-Dystroglycan Functions In Acetylcholine Receptor Aggregation But Is Not A Coreceptor For Agrin-MuSK Signaling. J. Neurosci. 18:6340-6348.
Jacobson, C., P. D. Cote, S. G. Rossi, R. L. Rotundo, and S. Carbonetto. 2001. The Dystroglycan Complex Is Necessary For Stabilization Of Acetylcholine Receptor Clusters At Neuromuscular Junctions And Formation Of The Synaptic Basement Membrane. J. Cell Biol. 152:435-450.
Jennings-White, C. et al., Tetrahedron Lett (1982) 23:2533.
Jeon, D., Yang, Y. M., Jeong, M. J., Philipson, K. D., Rhim, H., and Shin, H. S. (2003). Enhanced learning and memory in mice lacking Na+/Ca2+ exchanger 2. Neuron 38, 965-976.
Ji, R.-R., C. M. Böse, C. Lesuisse, D. Qiu, J. C. Huang, Q. Zhang, and F. Rupp. 1998. Specific Agrin Isoforms Induce cAMP Response Element Binding Protein Phosphorylation In Hippocampal Neurons. J. Neurosci. 18:9695-9702.
Juhaszova, M., and Blaustein, M. P. (1997). Na+ pump low and high ouabain affinity alpha subunit isoforms are differently distributed in cells. Proc Natl Acad Sci USA 94, 1800-1805.
Kaplan, J. H. (2002). Biochemistry of Na,K-ATPase. Annu Rev Biochem 71, 511-535.
Katzung Basic and Clinical Pharmacology, Appleton and Lange, Norwalk, Conn., (1992).
Kohler and Milstein (1975), Nature 256, 495-497.
Koulen, P., L. S. Honig, E. L. Fletcher, and S. Kröger. 1999. Expression, Distribution And Ultrastructural Localization Of The Synapse-Organizing Molecule Agrin In The Mature Avian Retina. Eur. J. Neurosci. 11:4188-4196.
Lévi, S., R. M. Grady, M. D. Henry, K. P. Campbell, J. R. Sanes, and A. M. Craig. 2002. Dystroglycan Is Selectively Associated With Inhibitory GABAergic Synapses But Is Dispensable For Their Differentiation. J. Neurosci. 22:4274-4285.
Li, Z., Hilgenberg, L. G. W., O'Dowd, D. K., and Smith, M. A. (1999). Formation of functional synaptic connections between cultured cortical neurons from agrin-deficient mice. J Neurobiol 39, 547-557.
Li, Z., Massengill, J. L., O'Dowd, D. K., and Smith, M. A. (1997). Agrin gene expression in mouse somatosensory cortical neurons during development in vivo and in cell culture. Neuroscience 79, 191-201.
Lindeberg, J., et al., Genesis 40: 67-73 (2004).
Liu, I. H., Uversky, V. N., Munishkina, L. A., Fink, A. L., Halfter, W., and Cole, G. J. (2005). Agrin binds _-synuclein and modulates _-synuclein fibrillation. Glycobiology 15, 1320-1331.
Mann, S., and Kröger, S. (1996). Agrin is synthesized by retinal cells and colocalizes with gephyrin. Mol Cell Neurosci 8, 1-13.
Mantych, K. B., and A. Ferreira. 2001. Agrin Differentially Regulates The Rates Of Axonal And Dendritic Elongation In Cultured Hippocampal Neurons. J. Neurosci. 21:6802-6809.
Massengill, J. L., et al., J. Neurosci. 17: 3136-3147 (1997).
Morley, J. S., Trends Pharm Sci (1980) pp. 463-468.
Needleman and Wunsch, J. Mol. Biol. 48: 443-453 (1970).
Neumann, F. R., G. Bittcher, M. Annies, B. Shumacher, S. Kröger, and M. A. Ruegg. 2001. An Alternative Amino-Terminus Expressed In The Central Nervous System Converts Agrin To A Type II Transmembrane Protein. Mol. Cell. Neurosci. 17:208-225.
Nitkin, R. M., Smith, M. A., Magill, C., Fallon, J. R., Yao, M. Y.-M., Wallace, B. G., and McMahan, U. J. (1987). Identification of agrin, a synaptic organizing protein from Torpedo electric organ. J Cell Biol 105, 2471-2478.
O'Connor, L. T., Lauterborn, J. C., Gall, C. M., and Smith, M. A. (1994). Localization and alternative splicing of agrin mRNA in adult rat brain: transcripts encoding isoforms that aggregate acetylcholine receptors are not restricted to cholinergic regions. J Neurosci 14, 1141-1152.
O'Connor, L. T., Lauterborn, J. C., Smith, M. A., and Gall, C. M. (1995). Expression of agrin mRNA is altered following seizures in adult rat brain. Brain Res Mol Brain Res 33, 277-287.
Osol et al., eds., Remington's Pharmaceutical Sciences, 17th edition, Mack Publishing Co., Easton, Pa. (1990).
Peters, H. C., et al., Nat. Neurosci. 8: 51-60 (2005).
Raats, C. J., Bakker, M. A., Hoch, W., Tamboer, W. P., Groffen, A. J., van den Heuvel, L. P., Berden, J. H., and van den Born, J. (1998). Differential expression of agrin in renal basement membranes as revealed by domain-specific antibodies. J Biol Chem 273, 17832-17838.
Reist, N. E., Magill, C., and McMahan, U. J. (1987). Agrin-like molecules at synaptic sites in normal, denervated, and damaged skeletal muscles. J Cell Biol 105, 2457-2469.
Reist, N. E., Werle, M. J., and McMahan, U. J. (1992). Agrin released by motor neurons induces the aggregation of acetylcholine receptors at neuromuscular junctions. Neuron 8, 865-868.
Reuter, H. & Porzig, H., Neuron 15, 1077-84 (1995).
Remington's Pharmaceutical Sciences, 16th Edition, Mack, (1980).
Ross, S. T., and Soltesz, I. (2001). Long-term plasticity in interneurons of the dentate gyrus. Proc Natl Acad Sci USA 98, 8874-8879.
Ruegg, M. A., K. W. K. Tsim, S. E. Horton, S. Kröger, and U. J. McMahan. 1992. The Agrin Gene Codes For A Family Of Basal Lamina Proteins That Differ In Function And Distribution. Neuron. 8:691-699.
Rupp, F., T. H. Özçelik, M. Linial, K. Peterson, U. Francke, and R. Scheller. 1992. Structure and Chromosomal Localization Of The Mammalian Agrin Gene. J. Neurosci. 12:3535-3544.
Sambrook, et al., MOLECULAR CLONING. A LABORATORY MANUAL (Cold Spring Harbor Press, 1989).
Sanes, J. R., and Lichtman, J. W. (2001). Induction, assembly, maturation and maintenance of a postsynaptic apparatus. Nat Rev Neurosci 2, 791-805.
Sato, T., Tanaka, K., Ohnishi, Y., Teramoto, T., Irifune, M., and Nishikawa, T. (2004). Effects of steroid hormones on (Na+, K+)-ATPase activity inhibition-induced amnesia on the stepthrough passive avoidance task in gonadectomized mice. Pharmacol Res 49, 151-159.
Serpinskaya, A. S., Feng, G., Sanes, J. R, and Craig, A. M. (1999). Synapse formation by hippocampal neurons from agrin-deficient mice. Dev Biol 205, 65-78.
Spatola, A. F. in “Chemistry and Biochemistry of Amino Acids, Peptides, and Proteins,” B. Weinstein, eds., Marcel Dekker, New York, p. 267 (1983).
Spatola, A. F., Vega Data (March 1983), Vol. 1, Issue 3, “Peptide Backbone Modifications”.
Smith, M. A. & Hilgenberg, L. G., Neuroreport 13, 1485-1495 (2002).
Spatola, A. F. et al., Life Sci (1986) 38:1243-1249 (—CH₂S—).
Sugiyama, J., D. C. Bowen, and Z. W. Hall. 1994. Dystroglycan Binds Nerve And Muscle Agrin. Neuron. 13:103-115.
Szelke, M. et al., European Appln. EP 45665 (1982) CA: 97:39405 (1982) (—CH(OH)CH₂—).
Tian, M., C. Jacobson, S. H. Gee, K. P. Campbell, S. Carbonetto, and M. Jucker. 1996. Dystroglycan In The Cerebellum Is A Laminin Alpha 2-Chain Binding Protein At The Glial-Vascular Interface And Is Expressed In Purkinje Cells. Eur. J. Neurosci. 8:2739-2747.
Vaillend, C., Mason, S. E., Cuttle, M. F., and Alger, B. E. (2002). Mechanisms of neuronal hyperexcitability caused by partial inhibition of Na+-K+-ATPases in the rat CAl hippocampal region. J Neurophysiol 88, 2963-2978.
Valenzuela, D. M., Stitt, T. N., DiStefano, P. S., Rojas, E., Mattsson, K., Compton, D. L., Nunez, L., Park, J. S., Stark, J. L., Gies, D. R., et al. (1995). Receptor tyrosine kinase specific for the skeletal muscle lineage: expression in embryonic muscle, at the neuromuscular junction, and after injury. Neuron 15, 573-584.
Veber and Freidinger (1985) TINS p. 392.
Verbeek, M. M., Otte-Holler, I., van den Born, J., van den Heuvel, L. P., David, G., Wesseling, P., and de Waal, R. M. (1999). Agrin is a major heparan sulfate proteoglycan accumulating in Alzheimer's disease brain. Am J Pathol 155, 2115-2125.
Wang, J., Schwinger, R. H., Frank, K., Muller-Ehmsen, J., Martin-Vasallo, P., Pressley, T. A., Xiang, A., Erdmann, E., and McDonough, A. A. (1996). Regional expression of sodium pump subunits isoforms and Na⁺—Ca⁺⁺ exchanger in the human heart. J Clin Invest 98, 1650-1658.
Williams and Wilkins, Baltimore, Md. (1987).
Xia, S., Liu, L., Feng, C., and Guo, A. (1997). Drug disruption of short-term memory in Drosophila melanogaster. Pharmacol Biochem Behav 58, 727-735.
Xiao, A. Y., Wei, L., Xia, S., Rothman, S., and Yu, S. P. (2002). Ionic mechanism of ouabaininduced concurrent apoptosis and necrosis in individual cultured cortical neurons. J Neurosci 22, 1350-1362.
Zaccaria, M. L., F. Di Tommaso, A. Brancaccio, P. Paggi, and T. C. Petrucci. 2001. Dystroglycan Distribution In Adult Mouse Brain: A Light And Electron Microscopy Study. Neuroscience. 104:311-324.
Zahler, R., Sun, W., Ardito, T., Zhang, Z.-t., Kocsis, J. D., and Kashgarian, M. (1996). The _{—}3 isoform protein of the Na+, K+-ATPase is associated with the sites of cardiac and neuromuscular impulse transmission. Circ Res 78, 870-879.
Zheng, J. Q. (2000). Turning of nerve growth cones induced by localized increases in intracellular calcium ions. Nature 403, 89-93.

### DEFINITIONS

The following terms are used herein:

“Individual” means any living organism, including humans and other mammals, which produce agrin.

“Native agrin” or “agrin” is an ˜400-kD heparan sulfate proteoglycan assembled on an ˜200-kD polypeptide backbone characterized by multiple cysteine-rich domains.

“C-Ag20” or “C-Ag20_{z0/z8}” refers to the 20-kD COOH-terminal fragment of agrin containing the alternatively spliced z site.

“C-Ag20_{z0}” refers to the C-Ag20 isoform having the z0 splice variant. The amino acid sequence of C-Ag20_{z0} is shown as SEQ. ID NO. 1; the nucleic acid sequence encoding C-Ag20_{z0} is shown as SEQ. ID NO. 4.

“C-Ag20_{z8}” refers to the C-Ag20 isoform having the z8 splice variant. The amino acid sequence of C-Ag20_{z8} is shown as SEQ. ID NO. 2; the nucleic acid sequence encoding C-Ag20_{z8} is shown as SEQ. ID NO. 5.

“C-Ag15” refers to the 15-kD COOH-terminal fragment of agrin created by deleting 37 amino acids from the NH₂ terminus of C-Ag20. The amino acid sequence of C-Ag15 is shown as SEQ. ID NO. 3; the nucleic acid sequence encoding C-Ag15 is shown as SEQ. ID NO. 6.

“Homologs” refers to polypeptides in which one or more amino acids have been replaced by different amino acids, such that the resulting polypeptide is at least 75% homologous, and preferably at least 85% homologous, to the basic sequence as, for example, the sequence of agrin, C-Ag20 or C-Ag15, and wherein the variant polypeptide retains the activity of the basic protein, for example, agrin, C-Ag20 or C-Ag15. Homology is defined as the percentage number of amino acids that are identical or constitute conservative substitutions. Conservative substitutions of amino acids are well known in the art. Representative examples are set forth in Table 1.

TABLE 1

| Original Residue | Conservative Substitution(s) |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Homologs of polypeptides may be generated by conventional techniques, including either random or site-directed mutagenesis of DNA encoding the basic polypeptide. The resultant DNA fragments are then cloned into suitable expression hosts such as E. coli or yeast using conventional technology and clones that retain the desired activity are detected. The term “homolog” also includes naturally occurring allelic variants.

“Derivative” refers to a polypeptide that has been derived from the basic sequence by modification, for example by conjugation or complexing with other chemical moieties or by post-translational modification techniques as would be understood in the art. Such derivatives include amino acid deletions and/or additions to polypeptides or variants thereof wherein said derivatives retain activity of the basic protein, for example, agrin, C-Ag20 or C-Ag15. Other derivatives contemplated by the invention include, but are not limited to, modification to side chains, incorporation of unnatural amino acids and/or their derivatives during peptide, polypeptide or protein synthesis and the use of crosslinking agents.

In addition to polypeptides consisting only of naturally-occurring amino acids, the present invention includes peptidomimetics. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986); Veber and Freidinger (1985); and Evans et al. (1987)) and are usually developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), but have one or more peptide liNa+/K+-ATPaseges optionally replaced by a liNa+/K+-ATPasege selected from the group consisting of: —CH₂NH—, —CH₂S—, —CH₂CH₂—, —CH═CH—(cis and trans), —COCH₂—, —CH(OH)CH₂—, and —CH₂SO—, by methods known in the art and further described in the following references: Spatola, A. F. (1983); Spatola, A. F., (March 1983) (general review); Morley, J. S., (general review); Hudson, D. et al., (1979) (—CH₂NH—, —CH₂CH₂—); Spatola, A. F. et al., (1986) (—CH₂S—); Hann, M. M., (1982) (—CH═CH—, cis and trans); Almquist, R. G. et al., (1980) (—COCH₂—); Jennings-White, C. et al., (1982) (—COCH₂—); Szelke, M. et al., (1982) (—CH(OH)CH₂—); Holladay, M. W. et al., (1983) (—C(OH)CH₂—); and Hruby, V. J., (1982) (—CH₂S—). A particularly preferred non-peptide liNa+/K+-ATPasege is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) (e.g., receptor molecules) to which the peptidomimetic binds to produce the therapeutic effect. Derivitization (e.g., labeling) of peptidomimetics should not substantially interfere with the desired biological or pharmacological activity of the peptidomimetic.

“Therapeutic composition” is defined as compounds that have been identified in drug screening assays as eliminating or ameliorating the effects of a disease, such as Parkinson's or a pathology, such as Parkinson's-related pathologies. Any such compounds, such as for example, C-Ag20 or C-Ag15 these compounds can be used as therapeutic agents, provided they are biocompatible with the animals, preferably humans, to whom they are administered. The therapeutic agents of the present invention can be formulated into pharmaceutical compositions by combination with appropriate pharmaceutically acceptable carriers or diluents, and may be formulated into preparations in solid, semisolid, liquid or gaseous forms, such as tablets, capsules, powders, granules, ointments, solutions, suppositories, injections, inhalants, gels, microspheres, and aerosols. Administration of the compounds can be administered in a variety of ways known in the art, as, for example, by oral, buccal, rectal, parenteral, intraperitoneal, intradermal, transdermal, intratracheal, etc., administration.

A “pharmaceutically acceptable carrier” is a solid or liquid filler, diluent or encapsulating substance that may be safely used in systemic administration. Depending upon the particular route of administration, a variety of pharmaceutically acceptable carriers, well known in the art can be used. These carriers include, but are not limited to, sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water. Preservatives and other additives can also be present. For example, antimicrobial, antioxidant, chelating agents, and inert gases can be added (see, generally, Remington's Pharmaceutical Sciences, (1980)).

Where a particular polypeptide or nucleic acid molecule is said to have a specific percent identity or conservation to a reference polypeptide or nucleic acid molecule, the percent identity or conservation can be determined by the algorithm of Myers and Miller, CABIOS (1989), which is embodied in the ALIGN program (version 2.0), or its equivalent, using a gap length penalty of 12 and a gap penalty of 4 where such parameters are required. All other parameters are set to their default positions. Access to ALIGN is readily available. See, e.g., http://www2.igh.cnrs.fr/bin/align-guess.cgi on the internet.

Comparison of the sequence to the databases can be performed using BLAST (Altschcul; S. F. et al., (1990)).

Parameters for polypeptide sequence comparison include the following: (1) Algorithm: Needleman and Wunsch, (1970); (2) Comparison matrix: BLOSSUM62 from Hentikoff and Hentikoff, (1992); (3) Gap Penalty: 12; and (4) Gap Length Penalty: 4. A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: (1) Algorithm: Needleman and Wunsch, J. Mol. Biol. 48:443-453 (1970); (2) Comparison matrix: matches=+10, mismatch=0; (3) Gap Penalty: 50; and (4) Gap Length Penalty: 3. Available as: The “gap” program from Genetics Computer Group, Madison Wis. These are the default parameters for nucleic acid comparisons.

Polypeptides of the invention may be prepared by any suitable procedure known to those of skill in the art. Recombinant polypeptides of the invention may be produced by culturing a host cell transformed with an expression vector containing nucleic acid encoding a polypeptide, fragment, homolog or derivative according to the invention. Recombinant protein may be conveniently prepared by a person skilled in the art using standard protocols as, for example, described in Sambrook, et al., (1989), in particular Sections 16 and 17; Ausubel et al., (1994-1998), in particular Chapters 10 and 16; and Coligan et al., (1995-1997), in particular Chapters 1, 5 and 6. Examples of vectors suitable for expression of recombinant protein include but are not limited to pGEX, pET-9d, pTrxFus or baculovirus (available from Invitrogen). A number of other vectors are available for the production of protein from both full length and partial cDNA and genomic clones, producing both fused or non-fused protein products, depending on the vector used. The resulting proteins are frequently immunologically and functionally similar to the corresponding endogenous proteins.

The obtained polypeptide is purified by methods known in the art. The degree of purification varies depending on the use of the polypeptide. For use in eliciting polyclonal antibodies, for example, the degree of purity may not need to be very high. However, as in some cases impurities may cause adverse reactions, purity of 90-95% is typically preferred and in some instances even required. For the preparation of a therapeutic composition, however, the degree of purity must be high, as is known in the art.

The present invention provides for the administration of a therapeutic composition comprising C-Ag20 or C-Ag15, or homologs, derivatives or peptidomimetics thereof, to an individual diagnosed with epilepsy, traumatic injury or other pathologies of the brain, such as Parkinson's or Parkinson's-related pathologies. In a preferred embodiment of the present invention, therapeutic compositions comprising C-Ag15, or homologs, derivatives or peptidomimetics thereof, are administered to inhibit agrin function to thereby control seizures associated with epilepsy, traumatic brain injury, and other disorders of the central nervous system in which agrin is shown to affect biological activity. In another preferred embodiment of the present invention, therapeutic compositions comprising C-Ag20, or homologs, derivatives or peptidomimetics thereof, are administered to rescue an agrin-deficient phenotype.

It is understood that the dosage of the therapeutic composition of the present invention administered in vivo or in vitro will be dependent upon the age, sex, health, and weight of the recipient, kind of concurrent treatment, if any, frequency of treatment, and the nature of the pharmaceutical effect desired. The most preferred dosage will be tailored to the individual subject, as is understood and determinable by one skilled in the relevant arts. See, e.g., Berkow et al., eds., (1992); Goodman et al., eds., (1990); Avery's Drug Treatment: Principles and Practice of Clinical Pharmacology and Therapeutics, (1987); Ebadi, (1985); Osol et al., eds., (1990); Katzung Basic and Clinical Pharmacology, (1992).

The term “therapeutically effective amount” as used herein, means that amount of C-Ag20 or C-Ag15 that elicits the biological or medicinal response in a tissue system, animal or human that is being sought by a researcher, veterinarian, medical doctor or other clinician, which includes alleviation of the symptoms of the disease or disorder being treated and may vary from about 0.01-100 wt. %.

The total dose required for each treatment can be administered by multiple doses or in a single dose. The diagnostic/pharmaceutical compound or composition can be administered alone or in conjunction with other diagnostics and/or pharmaceuticals directed to the pathology, or directed to other symptoms of the pathology.

As discussed above, the therapeutic composition of the invention may be administered by any of the conventional routes of administration, including oral, rectal, parenteral, sublingual, buccal, intravenous, intra-articular, intramuscular, intra-dermal, subcutaneous, inhalational, intraocular, intraperitoneal, intracerebroventricular, transdermal and the like, or as described in U.S. Pat. No. 5,693,607, the entire contents of which is hereby incorporated by reference. Also, the therapeutic composition of the invention may be in any of several conventional dosage forms, including, but not limited to, tablets, dispersions, suspensions, injections, solutions, capsules, suppositories, aerosols, and transdermal patches.

The invention also includes recombinant DNA vectors containing a gene encoding agrin, or fragments or variants thereof, preferably vectors that target neuronal cells, as, for example, by targeting overexpressed cell surface receptors.

The invention also contemplates polyclonal, monoclonal and humanized antibodies against the aforementioned agrin polypeptides, fragments, homologs and derivatives.

Methods of producing polyclonal antibodies are well known to those skilled in the art. Exemplary protocols which may be used are described for example in Coligan et al., (1991) which is incorporated herein by reference, and Ausubel et al., (1994-1998), in particular Section III of Chapter 11.

Alternatively, monoclonal antibodies may be produced using the standard method as, for example, described in an article by Kohler and Milstein (1975) which is herein incorporated by reference.

According to the method of the current invention, large amounts of recombinant agrin, or derivatives, homologs or fragments thereof, are produced by scale up processes in commercial plants which enables production of a corresponding large quantity of antibodies. The antibodies to recombinant expressed protein can also be produced according to the invention using the standard method available for production of the antibodies to native protein.

The antibodies of the invention may be used for affinity chromatography in isolating natural or recombinant agrin polypeptides or fragments thereof. The antibodies can also be used to screen expression libraries for variant polypeptides of agrin. Preferably, the antibodies of the invention can be administered to individuals diagnosed with epilepsy, traumatic injuries and other pathologies of the brain. Preferably, humanized antibodies (XENOMOUSE®, Abgenix, Inc., Fremont, Calif.; Bodey B., et al., (2000); Halloran P. F., et al., (1998)) are administered as therapeutic agents to treat epilepsy, traumatic injuries and other pathologies of the central nervous system.

Antibodies may be administered as described above for therapeutic compositions. Preferably, therapeutic antibodies are administered either subcutaneously or by intravenous

Those of skill will readily appreciate that dose levels can vary as a function of the specific therapeutic agents, the severity of the symptoms and the susceptibility of the subject to side effects. Preferred dosages for a given therapeutic agent are readily determinable by those of skill in the art by a variety of means. A preferred means is to measure the physiological potency of a given therapeutic agent

General Overview

Agrin has been implicated in a wide range of functions in central and peripheral neurons including organization of pre- and postsynaptic specializations, process growth, calcium homeostasis, and now neuronal activity. However, a general mechanism of agrin action has been elusive, in large part due to a lack of knowledge concerning the identity of the receptor(s) on neurons that bind(s) agrin. The current invention is based on the discovery that agrin acts as an endogenous ouabain-like molecule targeted specifically to the α3Na+/K+-ATPase, a member of the Na+/K+-ATPase family selectively expressed in neurons. Among other things, Na+/K+-ATPases are responsible for maintaining the Na+/K+ ion gradient that underlies the membrane potential and provides the driving force for a variety of secondary cellular processes necessary for normal cell function. The current invention provides screening tools and treatments for a wide range of disorders resulting from improper NA+/K+-ATPase pump function, based on the discovery that many of agrin's effects in neurons are driven by local and/or global changes in the Na+/K+ ion gradient.

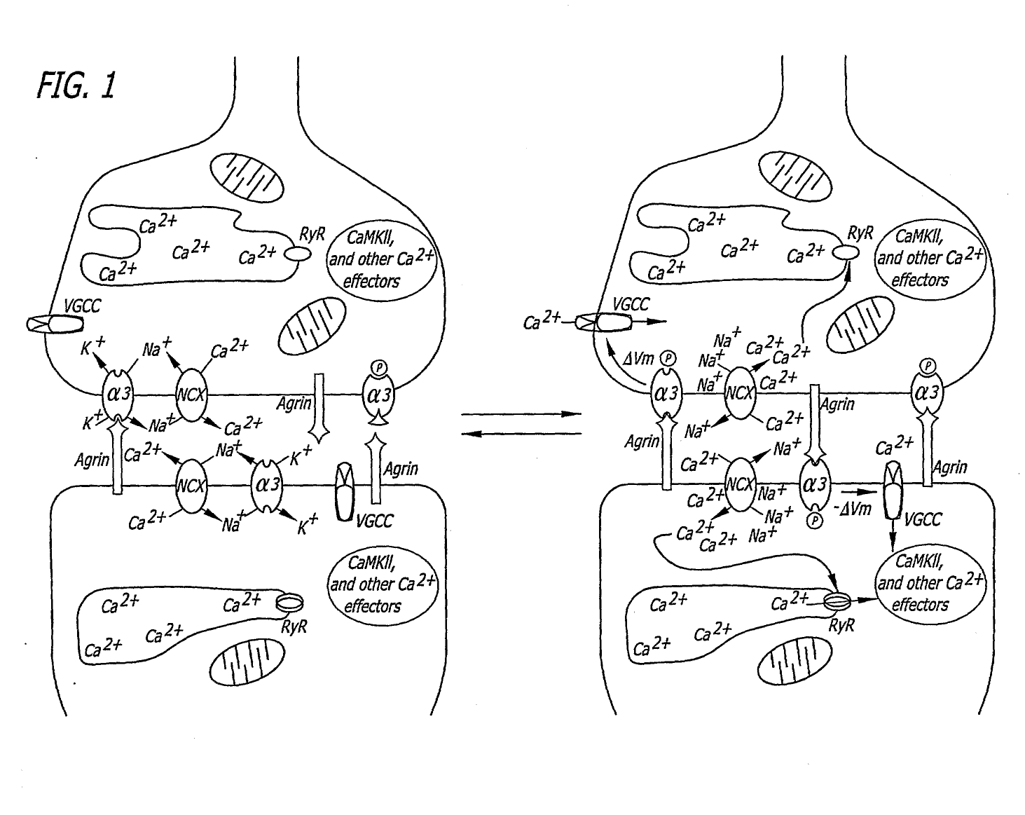

For example, an early response to agrin is an increase in cytoplasmic Ca2+, a composite of Ca2+ release from intracellular stores and influx through voltage-gated channels (Hilgenberg and Smith, 2004). The finding that agrin antagonizes the α3Na+/K+-ATPase provides a simple explanation for these observations (see model, FIG. 1). It is well known that the plasma membrane sodium/calcium exchanger (NCX) plays a key role in Ca2+ homeostasis. However, because of its dependence on the Na+ ion gradient, activity of the NCX is largely governed by the Na+/K+-ATPase: under normal conditions the pump operates in forward mode, transporting Ca2+ ions out of the cell, but the direction of transport reverses as the Na+ ion gradient declines (Annunziato et al., 2004). In neurons, the α3Na+/K+-ATPase and NCX colocalize within plasma membrane domains, juxtaposed by elements of the endoplasmic reticulum, creating a diffusion restricted cytoplasmic space that enhances the functional liNa+/K+-ATPasege between them (Blaustein et al., 2002). Thus, one component of the agrin induced increase in intracellular Ca2+ is due to changes in NCX activity driven by inhibition of the α3Na+/K+-ATPase. The results of the current invention shows the opening of voltage-gated Ca2+ channels in response to membrane depolarization associated with the agrin-induced decline in α3Na+/K+-ATPase activity, providing the possibility of creating screening and treatment techniques for disorders based on disorders in these channels.

The present invention further identifies a role for agrin in regulating neuronal activity. For example, inventors show surprisingly that action potential frequencies were dramatically increased by exogenous agrin; more importantly, C-Ag15, an agrin fragment that acts as an agrin antagonist, is shown to disrupt native agrin-α3Na+/K+-ATPase interactions, blocking spontaneous action potentials in both cultured neurons and acute slice preparations. Accordingly, it is shown that agrin behaves as an endogenous ouabain-like molecule. In turn, mechanisms of ouabain-induced hyperexcitabilty have been studied in hippocampal neurons where changes in both intrinsic membrane properties and synaptic transmission are important (Vaillend et al., 2002). For example, neurotransmitter release and/or spike threshold are both dependent on membrane potential; and so the functional coupling between the α3Na+/K+-ATPase and NCX, which plays a role in vesicle cycling and neurotransmitter release (Bouron and Reuter, 1996), is also important.

Consistent with the latter possibility, suppression of agrin expression in cultured hippocampal neurons is associated with a decrease in synaptic vesicle cycling (Böse et al., 2000). Not surprisingly, behavioral studies have shown that ouabain inhibits memory formation (Gibbs and Ng, 1978; Xia et al., 1997; Sato et al., 2004) while a decline in Na+/K+-ATPase activity is responsible for a form of long-term plasticity in hippocampal interneurons (Ross and Soltesz, 2001). Agrin expression is activity dependent (O'Connor et al., 1995) and it would appear that agrin regulation of the α3Na+/K+-ATPase plays a role in synaptic plasticity. Given the functional link between the α3Na+/K+-ATPase and NCX, studies showing enhanced learning and memory in mice lacking NCX2 (Jeon et al., 2003) support this hypothesis. Accordingly, the current invention also provides screening and treatment methods for addressing disorders associated with synaptic plasticity.

Dysfunction of the α3Na+/K+-ATPase has also been strongly linked with pathological changes in the brain. Intraventricular infusion of ouabain causes seizures (Davidson et al., 1978) and loss of α3Na+/K+-ATPase activity potentiates excitotoxic injury and neuronal cell death (Brines et al., 1995; Xiao et al., 2002). In addition, mutation of the α3Na+/K+-ATPase has been shown to be responsible for rapid-onset dystonia parkinsonism, an autosomal dominant movement disorder in human (de Carvalho Aguiar et al., 2004). Paralleling these observations, heterozygous agrin deficient mice are less sensitive to kainic acid than their wild type siblings, while agrin-deficient neurons exhibit decreased responses to excitatory stimuli and resistance to excitotoxic insult (Hilgenberg et al., 2002). Interestingly, these effects of mutating the agrin gene are predicted by the current model: decreased agrin expression, functionally equivalent to treatment with C-Ag15, translates into an increase in total α3Na+/K+-ATPase activity that lowers neuronal activity while enhancing the ability to buffer potentially damaging increases in intracellular Ca2+ ions by sustaining activity of the NCX. Together, these studies indicate that the dysregulation of agrin expression via the inventive methods will have a significant impact on brain function.

It is noteworthy that agrin is concentrated in both amyloid plaques and tangles characteristic of Alzheimer's disease (Verbeek et al., 1999) and Leowy bodies found in Parkinson's disease (Liu et al., 2005) and may, therefore, contribute to the etiology of these diseases. The ability of C-Ag15 to relieve inhibition of the α3Na+/K+-ATPase by endogenous agrin suggests it will be a useful starting point for the development of therapeutic agents that might alleviate or reverse the progress of these and other diseases of the CNS.

Finally, agrin was originally identified at the neuromuscular junction where it mediates the motor neuron induced accumulation of AChR in the postsynaptic muscle fiber membrane. Curiously, agrin molecules present at the junction are functionally heterogeneous and distinct in cellular origin: alternatively spliced z+ isoforms, equivalent to C-Ag908 and C-Ag208, have high AChR clustering activity and originate from motor neurons; z0 agrin, like C-Ag200, has no AChR clustering activity and is synthesized by muscle (Sanes and Lichtman, 2001). Although the function of z0 agrin is unclear, its location and origin are consistent with a role as a retrograde signal agent. The fact that the α3Na+/K+-ATPase is expressed on motor neuron axon terminals (Zahler et al., 1996) indicates it is a target for muscle agrin. Possible roles for this retrograde signal would include tuning neurotransmitter release to the muscle fiber's action potential threshold or matching growth of the axon terminal to the muscle fiber. In turn, guidance of developing axons is known to depend on translation of local cues to changes in intracellular Ca2+ within the growth cone (Zheng, 2000), which are also sites of α3Na+/K+-ATPase concentration (Brines and Robbins, 1993). Thus, agrin regulation of α3Na+/K+-ATPase provides an opportunity to address disorders where motor neurons overgrow their target muscle, such as has been studied in agrin mutant mice (Gautam et al., 1996), and where z0 agrin inhibits growth and stimulates axon terminal differentiation in cultured neurons (Campagna et al., 1997).

Finally, because of its positive inotropic effect, another major therapeutic use of ouabain and related compounds is in the treatment of congestive heart failure. Although cardiac muscle expresses multiple Na+/K+-ATPase isoforms, the low effective dose of ouabain suggests its therapeutic effects are mediated by the high affinity α2- and α3Na+/K+-ATPases (Glitsch, 2001). Agrin, which acts as an endogenous ouabain, is also expressed in heart (Godfrey et al., 1988; Hoch et al., 1993), indicating that it may also be useful in improved therapies for cardiac disease.

Materials and Methods: Tissue and Cell Cultures.

Mouse cortical cultures were prepared from newborn or 1-d-old ICR strain mice (Harlan) as described previously (Hilgenberg et al., 1999). For the first 24 h after plating, cells were maintained in neural basal medium (NBM) plus B27 supplements (Invitrogen), and in nonneuronal cell-conditioned NBM (cNBM) plus B27 thereafter, at 37° C. in humidified 5% CO₂ atmosphere. To further reduce proliferation of nonneuronal cells, cultures on glass coverslips used for histology were treated with 5 μM 5-fluoro-2-deoxyuridine (Sigma-Aldrich) 3-4 d after plating. Hippocampal and cerebellar cultures were prepared in a similar manner. Experiments were performed on 10-14-d-old cultures. Agrin-deficient neuron cultures were prepared from cortices of embryonic d 18-19 fetuses resulting from matings between mice heterozygous for a mutation in the agrin gene (Gautam et al., 1996). Cultures were prepared and genotyped as described previously (Li et al., 1999), and were maintained as above.

Chick muscle cultures were prepared from pectoral muscles of 10-11-d-old White Leghorn chick embryos as described previously (Hilgenberg et al., 1999). Experiments were performed on 4-6-d-old cultures.

Primary cultures of mouse 1-2 day postnatal cortical neurons, were prepared as described (Li, Z., et al., (1999)). Biochemical studies were performed on cells grown in 100 mm culture dishes. For Na⁺ imaging, neurons were plated on glass windowed 35 mm culture dishes (Matteck Corp.). Immunohistological studies and electrophysiological recordings were carried out on cells grown on glass coverslips. Experiments were performed between 10-16 days in culture. All handling and treatment of animals complied with the guidelines of the Institutional Animal Care and Use Committee of the University of California at Irvine.

Materials and Methods: Expression Constructs.

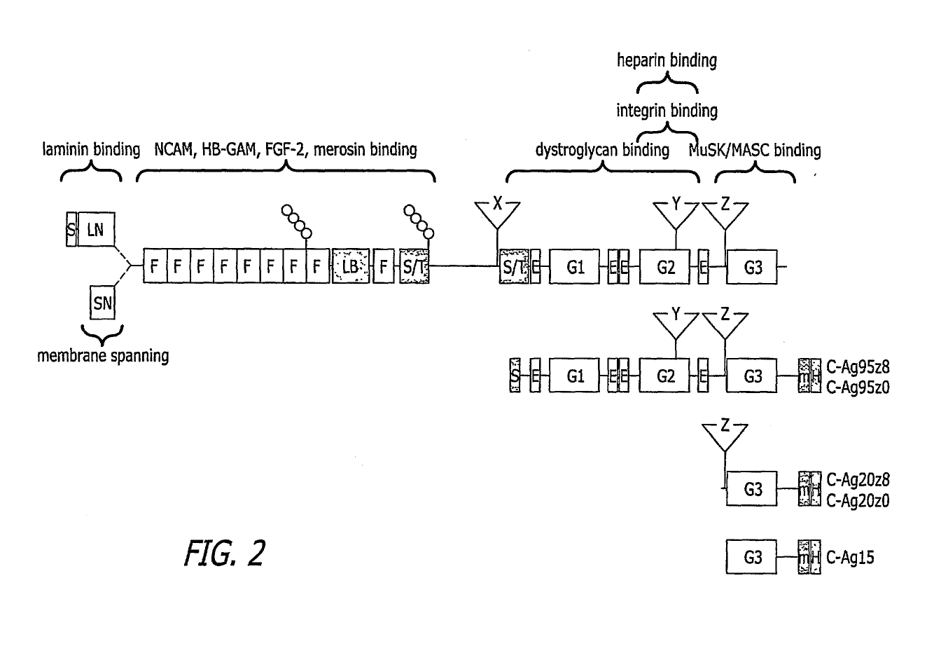

Parent constructs, C-Ag95_{z0} and C-Ag95_{z8} (FIG. 2), encoding the soluble 95-kD COOH terminus of mouse agrin, were generated from cDNA prepared by RT-PCR of adult mouse cortex RNA using the F95/R95 primer pair (see Table 2) subcloned into the pGEM-T (Promega) shuttle vector and transformed into JM109-competent bacteria. Individual ampicillin-resistant colonies were picked, and C-Ag95_{z0}, and C-Ag95_{z8}-containing clones were identified by PCR analysis using primers F24/B2 flanking the z site. After double digestion of the BamHI and EcoRI linker sites contained within the F95 and R95 primers, agrin inserts were gel purified and ligated into the pSecTag2B expression vector (Invitrogen) in frame with a COOH terminal myc epitope and 6× polyhistidine tag.

C-AgΔ20 lacking the 20-kD COOH-terminal region of C-Ag_{z0/8} was prepared by PCR amplification of the C-Ag95_{z8} pGEM-T template using F95 and the reverse primer RΔ20 that includes a 3′ EcoRV site. DNA from the PCR reaction was digested with BamHI and EcoRV, gel purified, and ligated into pSecTag2B.

A similar strategy was used to generate C-Ag20_{z0/8} and C-Ag15 constructs by PCR amplification of the appropriate pGEM-T C-Ag_{z0/8} template using either F20 (for C-Ag20_{z0/8}) or F15 (for C-Ag15) in combination with R20. However, for more efficient expression, aliquots of the PCR reaction were ligated into the inducible bacterial expression vector pTrcHis2 (Invitrogen).

TABLE 2

| | | | Nucleo- |
|---|---|---|---|
| Construct | Primer | Sequence | tide |
| C-Ag_{y4z0/8} | F95 | TAGGATCCACCGCCAGTATTGA | 1104-1121 |
| | | CCGA | |
| | R95 | TAGATATCAGAGTGGGGCAGGG | 6678-6663 |
| | | TCTT | |
| C-AgΔ20 | RΔ20 | TAGATATCGTCCGCCCATCAAA | 4585-4568 |
| | | GGCC | |
| C-Ag20_{z0/8} | F20 | AATGGATCCTCGGACCTACATC | 4581-4593 |
| | | G | |
| | R20 | TTCGAATTCAGAGTGGGGCAGG | 6679-6666 |
| | | G | |
| C-Ag15 | F15 | AATGGATCCGTGGATTGGCAAG | 5750-5764 |
| | | GC | |

Materials and Methods: Expression and Quantitation of Recombinant Agrin.

pSecTag2B agrin vector DNA encoding either C-Ag_{z0/8} or C-AgΔ20 was transfected into HEK 293T cells using LipofectAMINE™ (Invitrogen) according to the manufacturer's directions. Controls were either sham transfected with LipofectAMINE™ alone or with control vector encoding prostate-specific antigen (pSecTag2-PSA). Agrin constructs in the pTrcHis2 expression vector were maintained in the JM109 bacteria. The plasmid pTrcHis2-lacZ encoding β-galactosidase was expressed as a control.

Polyhistidine-tagged agrin fragments were purified from conditioned media and bacterial extracts using the Talon™ (CLONTECH Laboratories, Inc.) metal affinity resin eluted with 200-500 mM imidazole (Sigma-Aldrich) according to the manufacturer's instructions. The identity of the isolated fragments was confirmed by immunoblot analysis using RαAg-1, a rabbit antiserum raised against a synthetic peptide corresponding to amino acids 1862-1895 conserved in all isoforms of mouse agrin. For some experiments, the elution buffer was removed by dialysis against PBS or 20 mM Tris and 250 mM NaCl, pH 8.0.

The molar concentration of each fragment was determined by comparison to a C-Ag95_{z0} standard prepared as follows: HEK 293T cells were transfected with pSecTag2B-C-Ag_{z0}, and were then transferred to 80% methionine-free DME containing 100 μCi/ml [³⁵S]methionine. C-Ag95_{z0} present in the medium was purified over a Talon™ metal affinity resin column, and the apparent molar concentration was determined by counting aliquots of the column eluate in a scintillation counter (model LS7500; Beckman Coulter). A small correction factor was applied to account for the fraction of counts incorporated into C-Ag95_{z0} (≧90%) versus total counts determined by phosphorimager analysis (Molecular Dynamics, Inc.) of an aliquot of the eluate separated on an 8% SDS-PAGE gel.

The concentration of the other agrin fragments was determined by comparison to a ³⁵S-labeled C-Ag95_{z0} standard in immunoblots probed with a mouse anti-myc antibody (Invitrogen) and ¹²⁵I-labeled antimouse second antibody (Amersham Biosciences). The amount of ¹²⁵I bound to both the standard and unknown was determined by phosphorimager analysis and, after correcting for the contribution of the [³⁵S]methionine in the standard, the concentration of the unknown was determined from the standard curve.

Soluble 95-kD COOH-terminal fragments of rat agrin (rC-Ag_{y4z8/y0z0}) were harvested in media conditioned by transiently transfected COS-7 cells (Hilgenberg et al., 1999) and dialyzed against PBS. The concentration of the rC-Ag was estimated by comparison to a mouse agrin standard in the c-fos induction assay and by immunoblot analysis with RαAg-1.

Materials and Methods: Biochemistry.

Membrane impermeant chemical cross-linking agents BS³ and DMA were used to stabilize the bond between agrin and its binding sites on cell surface membranes. Cultured neurons were washed briefly in PBS containing 10 mM EDTA followed by preincubation with one or more agrin fragment in PBS²⁺ for 30 minutes at room temperature and then cooled on ice prior to addition of a 10× solution of cross-linking agent to a final concentration of 0.1 mM. The cross-linking reaction was allowed to proceed for 30 minutes after which any unreacted cross-linker was quenched and removed by washing with ice cold PBS²⁺ containing 50 mM ethanolamine. For immunoblot analysis, cells were scraped into ice-cold TI buffer (20 mM Tris, pH 7.4; 10 mM EDTA; protease inhibitors (Sigma, P8340)) then Dounce homogenized and membrane fraction recovered by centrifugation. For the immunoprecipitation studies, cells were scraped and homogenized in TI buffer containing 150 mM NaCl and 0.5% Triton X-100. Cell extracts were cleared by centrifugation and aliquots of the detergent soluble fraction incubated with the appropriate antibody at 4° C. overnight. Antigen-antibody complexes were precipitated with either protein A or protein G and resuspended in SDS-PAGE sample buffer for immunoblot analysis.

Materials and Methods: Quantitative Analysis of Fos Expression.

Fos expression in cortical cultures was measured by in situ enzyme-linked immunoassay (Hilgenberg et al., 1999). In brief, 11-14-d-old neuronal cultures were treated for 10 min with agrin or other agent diluted in NBM or PBS, then washed in cNBM and returned to the incubator for 2 h. Cultures were rinsed in PBS, fixed in ice cold 4% PFA, and blocked in PBS containing 0.1% Triton X-100 and 4% BSA (PBSTB) before being incubated in a primary rabbit antibody against Fos (Ab-2; Oncogene Research Products) and secondary goat antibody against mouse conjugated to alkaline phosphatase (Southern Biotechnology Associates, Inc.). The level of Fos expression was determined by monitoring conversion of p-nitrophenyl phosphate to a soluble yellow reaction product at 405 nm.

Materials and Methods: AChR Clustering Assay.

4-6-d-old myotubes were treated with agrin overnight followed by incubation with 20 nM rhodamine-conjugated α-bungarotoxin (Molecular Probes, Inc.) in culture medium for 1 h at 37° C. Cells were then fixed in 4%® PFA in PBS, washed in PBS, and viewed at 200× under epifluorescent illumination on a microscope (Optiphot-2; Nikon). For each well, the mean number of AChR clusters/field was determined from counts obtained from five random fields. All counts were performed blind with respect to treatment. To facilitate comparison between experiments, the number of AChR clusters/field was normalized to the cluster density of control cultures treated with vehicle alone.

Materials and Methods: Fos Immunohistochemistry.

10-14-d-old cortical neurons, grown on glass coverslips, were treated with agrin, fixed, and blocked as for the Fos in situ enzyme-linked immunoassay. Cells were double labeled overnight at 4° C. with Ab-2 (1:200) together with either a mouse monoclonal antibody against MAP2 (SMI-52; 1:400; Sternberger Monoclonals), or GFAP (G-A-5; 1:1,000; Sigma-Aldrich) in PBSTB to identify neurons or glial cells, respectively. Bound antibodies were visualized by incubation for 2 h at RT in a mixture of fluorescein-conjugated goat antirabbit and Texas red-labeled goat antimouse secondary antibodies (Vector Laboratories) diluted 1:200 in PBSTB. Coverslips were washed in PBS, mounted in Fluoromount™ (Southern Biotechnology Associates, Inc.), and examined using epifluorescent illumination.

Materials and Methods: Agrin Immunohistochemistry.

Live cortical cultures on glass coverslips were incubated for 30 min at 37° C. in R□Ag-1 diluted 1:500 in NBM. Cultures were then washed in NBM, fixed and blocked as described for the Fos in situ enzyme-linked immunoassay, and were then washed and incubated overnight at 4° C. with the anti-synaptophysin antibody, SVP38 (Sigma-Aldrich), diluted 1:400 in PBS containing 4% BSA (PBSB). Primary antibodies were visualized by double labeling with fluorescein-conjugated goat anti-rabbit and Texas red-labeled anti-mouse antibodies as above.

Materials and Methods: Agrin Receptor Immunohistochemistry.

The distribution of agrin receptors was studied using agrin deletion fragments as affinity probes. Neurons, plated on glass coverslips, were washed briefly (1-2 min) in cold PBS containing 10 mM EDTA followed by a second wash in cold PBS alone before incubation for 15 min at 4° C. with 1 pM recombinant mouse agrin in NBM. In some experiments, labeling with mouse agrin was performed in the presence of various concentrations of rat rC-Ag95y4_{z8} or rC-Agy_{0z0}. Control cultures were treated with vector control protein or an equivalent volume of vehicle in which the recombinant agrin was dissolved. Cells were then washed in cold NBM, fixed for 40 min on ice in 4% PFA in PBS, then washed in PBS and blocked for 1 h in PBSB. Recombinant agrin was detected through the COOH-terminal 6× polyhistidine tag using an anti-his antibody (1:500; Invitrogen). In experiments where cells were double labeled for synaptophysin, the anti-synaptophysin:antiserum (1:400) was also added at this step. Incubations were performed overnight at 4° C. in PBSB, followed by labeling with fluorescein conjugated goat anti-mouse and Texas red labeled anti-rabbit antibodies as described in the previous paragraphs. In some experiments, the amount of mouse agrin bound was estimated by digital photomicrography using the public domain NIH Image program (http://rsb.info.nih.gov/nih-image/).

Materials and Methods: α3Na⁺/K⁺-ATPase Immunohistochemistry.

Neuronal and non-neuronal cells were identified by double staining with a mouse antibody directed against MAP-2 (SMI-52, Sternberger Monoclonals) and a rabbit antibody against GFAP (DAKO) as described (Hilgenberg, L. G. W., et. al., (1999)). Agrin binding sites and α3Na⁺/K⁺-ATPase were visualized by a modification of the method described by Hoover et al. (Hoover, et. al., (2003)). Briefly, neurons were washed for 5 minutes in cold phosphate buffered saline (PBS) containing 10 mM EDTA followed by incubation in a saturating concentration of C-Ag20₈ in PBS containing 1.8 mM Ca²⁺ (PBS²⁺) for 15 minutes. Cells were then washed in cold PBS²⁺, fixed in 4% paraformaldehyde in PBS for 40 minutes and blocked for 1 hour in PBS containing 4% bovine serum albumin followed by incubation with a rabbit antibody to the COOH-terminal polyhistidine tag on C-Ag20₈ (Affinity BioReagents) and mAb XVIF9-G10 to α3Na⁺/K⁺-ATPase. Bound C-Ag20₈ and XVIF9-G10 was visualized using Alexa Fluor- and fluorescein-conjugated anti-rabbit and anti-mouse antibodies, respectively (Molecular Probes).

Materials and Methods: Na⁺ Imaging.

Intracellular Na⁺ was monitored by ratiometric imaging of the membrane permeant sodium binding fluorescent dye SBFI-AM (Molecular Probes) by essentially the same methods described for Fura-2 imaging of agrin-induced changes in neuronal Ca²⁺ (Hilgenberg, et. al., (2004)). For quantitative analyses, responses of individual cells were normalized to their maximal response to treatment with 5 μM gramicidin, a potent ionophore. All experiments were performed in the presence of a cocktail of voltage- and ligand gated channel blockers containing 1 μM tetrodotoxin (TTX), 10 μM bicuculline methchloride (BMC), 100 μM d-tubocurare (dTbC), 50 μM DL-2-amino-5-phosphonovaleric acid (APV), and 10 μM 6-cyano-7-nitroquinoxaline-2,3-dione (CNQX).

Materials and Methods: Electrophysiology.

Neuron membrane potential was measured using whole cell current clamp recording techniques. Records were obtained in a bathing solution of Hepes buffered saline (HBS; 120 mM NaCl, 5.4 mM KCl, 0.8 mM MgCl₂, 1.8 mM CaCl₂, 15 mM glucose, 20 mM Hepes, pH7.4) containing TTX, BMC, dTbC, APV, and CNQX. Patch pipettes, filled with a solution containing 120 mM K-gluconate, 20 mM NaCl, 0.1 mM CaCl₂, 2.0 mM MgCl₂, 1.1 mM EGTA, 4 mM ATP, and 10 mM HEPES, had a tip resistance of 3-5 Ma Data were acquired using a List EPC7 amplifier and Digidata 1320A. D-A converter controlled by pClamp 8 (Axon Instruments) software and analyzed using Clampfit 9 (Axon Instruments). All recordings were made at room temperature. Only neurons with stable pre-treatment resting potentials of −55 mV or less that exhibited a reversible response to agrin or ouabain were accepted for analysis.

Materials and Methods: Methods of Making Transgenic Animals.

Transgenic animals can be produced by any suitable method known in the art, such as manipulation of embryos, embryonic stem cells, etc. Transgenic animals may be made through homologous recombination, where the endogenous locus is altered. Alternatively, a nucleic acid construct is randomly integrated into the genome. Vectors for stable integration include plasmids, retroviruses and other animal viruses, YACs, and the like.

Specific methods of preparing the transgenic animals of the invention as described herein. However, numerous methods for preparing transgenic animals are now known and others will likely be developed. See, e.g., U.S. Pat. Nos. 6,252,131, 6,455,757, 6,028,245, and 5,766,879, all incorporated herein by reference. Any method that produces a transgenic animal in which expression of agrin or the α3Na⁺/K⁺ATPase is disrupted or enhanced in neurons or other cells is suitable for use in the practice of the present invention.

Materials and Methods: Drug Screening Assays.

The transgenic animals described herein may be used to identify compounds useful in the treatment of Parkinson's disease and/or Parkinson's-related pathologies. For example, transgenic animals of the present invention may be treated with various candidate compounds and the resulting effect, if any, on motor performance evaluated. Preferably, the compounds screened are suitable for use in humans.

Drug screening assays in general suitable for use with transgenic animals are known. See, for example, U.S. Pat. Nos. 6,028,245 and 6,455,757, incorporated herein by reference. Immunoblot analyses, expression studies, measurement of agrin and agrin proteolytic fragments by ELISA, immunocytochemical and histological analysis and behavioral analyses suitable for use with the transgenic animal of the present invention are described herein. However, it will be understood by one of skill in the art that many other assays may also be used. The subject animals may be used by themselves, or in combination with control animals. For example, in one embodiment, the screen using the transgenic animals of the invention can employ any phenomena associated with Parkinson's disease or Parkinson's-related pathologies that can be readily assessed in an animal model.

### EXEMPLARY EMBODIMENTS

### Example 1

### Agrin Signaling in Neurons is Independent of Splicing at the z Site

Initial characterization of the agrin signal transduction pathway in CNS neurons demonstrated its inability to discriminate between “active” e and “inactive” f agrin isoforms (Hilgenberg et al., 1999). However, these works used alternatively spliced variants of the 95-kD COON-terminal region of rat agrin (rC-Ag_{z0/8}), and were limited by the fact that only indirect estimates of agrin concentration could be made, leaving open the possibility that some difference in the specific activities of alternatively spliced isoforms might have gone undetected. To address this issue directly, new 95-10 mouse agrin constructs (FIG. 2; C-Ag95_{z0/8}) were assembled in the pSecTag2 expression vector (Invitrogen), incorporating COOH-terminal myc and polyhistidine epitope tags, permitting purification and detection of the expressed protein and accurate concentration measurement to be made. (see Materials and Methods, above). Because the vast majority of agrin molecules expressed in brain include the 4 amino acid exon at the y site (Hoch et al., 1993; Li et al., 1997), all agrin constructs included the y4 exon, and only the properties of z-site variants were examined.

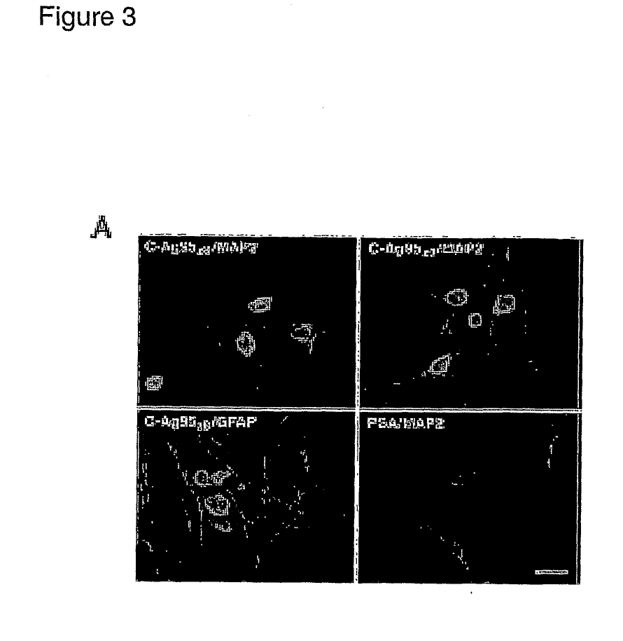

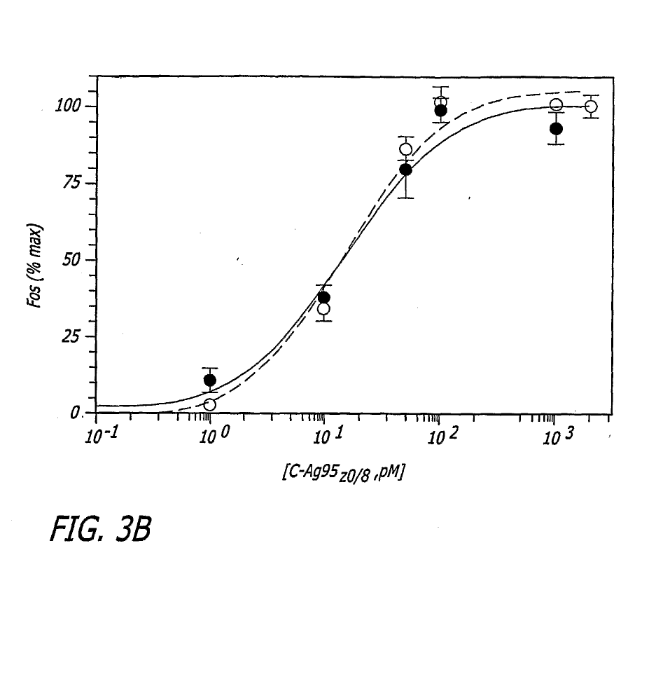

Rat rC-Agz0 and rC-Agz8 induce a neuron-specific increase in Fos expression (Hilgenberg et al., 1999). To confirm the properties of the corresponding mouse constructs, 12-d-old cortical cultures were treated with 1 nM purified mouse C-Ag95z0 or C-Ag95z8, and were then double labeled with antibodies against Fos and either microtubule-associated protein 2 (MAP2) or glial fibrillary acidic protein (GFAP) to identify neurons and glial cells, respectively. Consistent with previous results, treatment with either C-Ag95z0 or C-Ag95z8 caused a marked increase in Fos expression in neurons, but not nonneuronal cells (FIG. 3). Although differences in the level of Fos expression between neurons were apparent, virtually all neurons (>90%) responded to the C-Ag95z0/8 treatment. In contrast, treatment with a similar concentration of prostate serum antigen control protein expressed in the same vector had no effect on Fos levels in either neurons or glia (FIG. 3). In light of these results, it appears that neither the myc epitope nor polyhistidine tags induce c-fos, nor do they affect the ability of the C-Ag95z0/8 sequences to do so.

Next, the specific activity of each z-site isoform was determined using an in situ enzyme-linked immunoassay (Hilgenberg et al., 1999) to examine the concentration dependence of c-fos induction by C-Ag95z0 and C-Ag95z8. As shown in FIG. 2B, both agrin isoforms induced c-fos in a concentration-dependent and saturable fashion. Fos expression curves were well fit by a single-site nonlinear regression model (R2≧0.94) predicting EC50 values of 11.93±0.44 pM for C-Ag95z0 and 12.67±0.58 pM for C-Ag95z8 (mean±SEM). Similar EC50 values have also been reported for agrin induced clustering of AChR in muscle, but in contrast to the >1,000-fold difference in AChR clustering activity between isoforms (Ferns et al., 1992; Ruegg et al., 1992; Gesemann et al., 1995), the c-fos-inducing activity of C-Ag95z8 and C-Ag95z0 in neurons is the same.

### Example 2

### The 20-kD COOH-Terminal Portion of Agrin is Necessary and Sufficient for Signaling in Neurons

As a first step in localizing the structural domains responsible for signaling in neurons, the knowledge gained from previous structural analyses of agrin function in muscle (Hoch et al., 1994; Gesemann et al., 1995, 1996) was used and C-Ag95_{z0/8} was divided into two fragments. The first, an 75-kD NH 2-terminal fragment (FIG. 2; C-AgΔ20), which includes the 4 amino acid exon at the y site, has been shown to mediate agrin binding to α-dystroglycan (Gesemann et al., 1996; Hopf and Hoch, 1996). The remaining 20-kD COOH-terminal fragment contains the alternatively spliced z site (FIG. 2; C-Ag20_{z0} or C-Ag20_{z8}), and is homologous to the minimal agrin fragment able to induce clustering of AChR in cultured muscle cells (Gesemann et al., 1995).

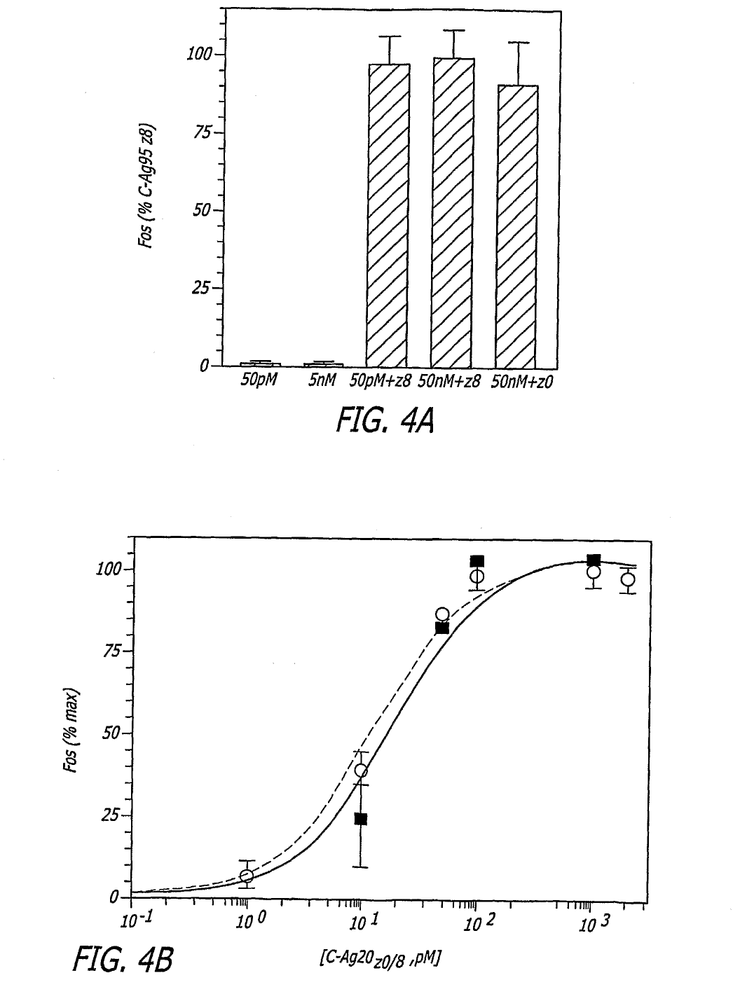

Treatment with C-AgΔ20, at a concentration equivalent to either a near saturating (50 pM) or supersaturating (5 nM) amount of C-Ag95_{z0/8}, had no effect on Fos expression in cortical cultures, suggesting that the active domain is not present within the C-AgΔ20 region (FIG. 4A). Agrin binding to α-dystroglycan has been shown to modulate agrin induced AChR clustering in muscle, and the possibility of a similar function for α-dystroglycan signaling in neurons was considered. However, coincubation with either an equal or 100-fold molar excess of C-AgΔ20 had no effect on Fos expression induced by either the C-Ag95_{z0} or C-Ag95_{z8} isoforms (FIG. 4A). Together, these data suggest that domains present within C-AgΔ20 are neither required for, nor modulate, agrin induction of c-fos in neurons.

The C-Ag20 fragments were both potent inducers of c-fos. As with C-Ag95_{z0/8}, c-fos induction by C-Ag20_{z0} (SEQ ID NO. 1) and C-Ag20_{z8} (SEQ ID NO. 2) was concentration-dependent and saturable (FIG. 4B). In fact, the EC₅₀ values obtained for the 20-10 fragments (C-Ag20_{z0}, 13.33±0.26 pM; C-Ag20_{z8}, 11.25±2.88 pM) were indistinguishable from each other and from those of the C-Ag95_{z0/8} isoforms. In light of these observations, it can be concluded that the structural domains that mediate agrin induction of c-fos are contained within the C-Ag20_{z0} fragment.

### Example 3

### Sequences Flanking the z Site are Critical for Agrin Signaling

Agrin's AChR clustering activity is regulated by alternative splicing at the z site.

However, the observation that a peptide of the 8 amino acid alternatively spliced insert is itself inactive (Gesemann et al., 1995) suggests that domains important for agrin's bioactivity in muscle include not only the z site, but also amino acids that flank it. Because inclusion of alternatively spliced exons at the z site has no effect on agrin activity in neurons, the role of sequences surrounding the z site were determined. To address this question, 37 amino acids were deleted from the NH₂ terminus of C-Ag20_{z0} to the border of the G3 domain, giving rise to a 15-kD COOH-terminal fragment, C-Ag15 (FIG. 2; SEQ. ID NO. 3).

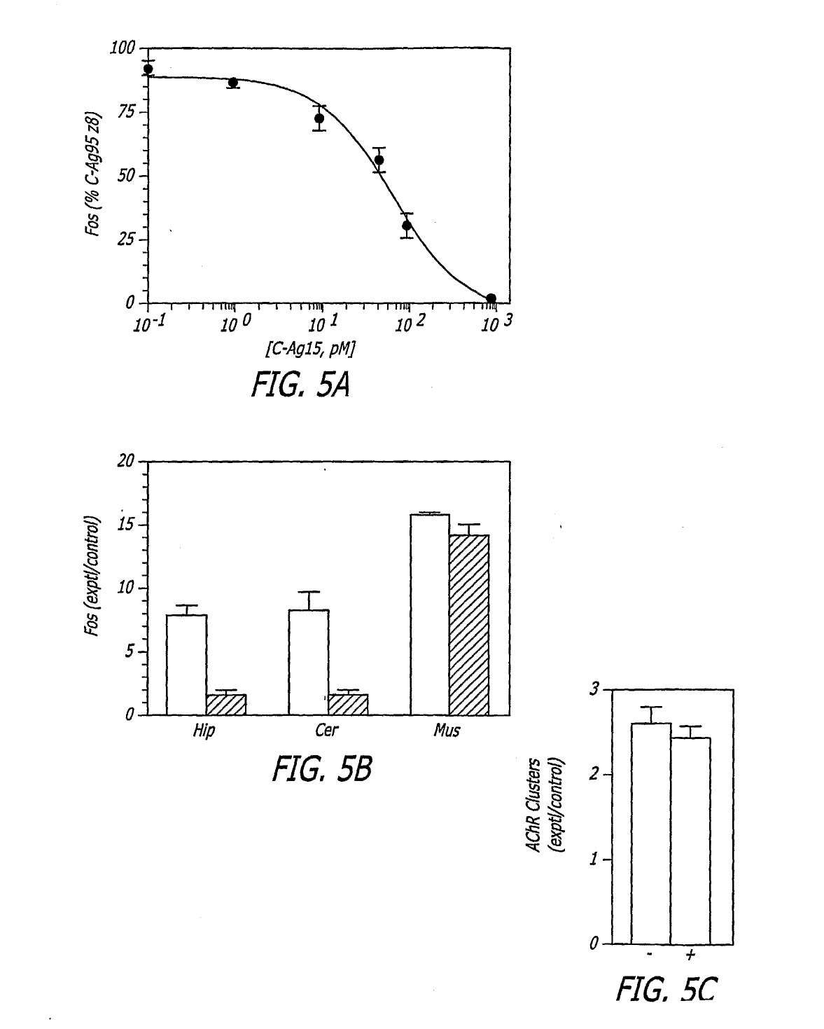

Treatment with C-Ag15 alone had no effect on the levels of Fos expression, even when present at a concentration fivefold above saturation for C-Ag20_{z0/8} (unpublished data). However, when added together with C-Ag95_{z8}, C-Ag15 appeared to inhibit the c-fos-inducing activity of the larger agrin fragment. To examine this effect in more detail, C-Ag15 dose-response studies were performed in the presence of a near saturating (50 pM) concentration of C-Ag95_{z8}. Increasing concentrations of C-Ag15 inhibited the c-fos-inducing activity of C-Ag95_{z8} (FIG. 5A). The curve was well fit by a nonlinear regression model for single-site competition (R²=0.95) predicting an IC₅₀ of 64.45±10.94 pM and close to a 1:1 agonist:antagonist molar ratio. Similar results were also obtained for competition against 50 pM C-Ag95_{z0} and the C-Ag20_{z0/8} isoforms (unpublished data).

To learn whether inhibition of agrin signaling by C-Ag15 might extend to other neurons or even muscle, mouse hippocampal and cerebellar neurons or chick skeletal myotubes were incubated with 50 pM C-Ag95_{z8} alone or in the presence of 1 nM C-Ag15, a concentration that blocks activity in cortical neurons. Treatment with C-Ag95_{z8} triggered a robust increase in Fos levels in both populations of neurons and in muscle compared with control sister cultures receiving vehicle (FIG. 5B). However, co-incubation with C-Ag15 inhibited Fos expression in the neurons, but had no effect on muscle. Because agrin's bioactivity in muscle is normally measured in terms of its AChR clustering activity, the ability of C-Ag15 to antagonize agrin-induced clustering of AChR was also examined. Chick myotubes were incubated overnight with 50 pM C-Ag95_{z8} alone or with 1 nM C-Ag15, AChR labeled with rhodamine-conjugated α-bungarotoxin, and the number of AChR clusters were determined. In line with the results of the Fos assay, C-Ag15 had no effect on the AChR clustering activity of C-Ag95_{z8} (FIG. 5C). Based on the results of these works, it appears that C-Ag15 is a specific antagonist for the neuronal receptor for agrin.

### Example 4

### C-Ag20z0/8 Rescues an Agrin-Deficient Neuronal Phenotype

Although induction of c-fos is a convenient reporter facilitating biochemical characterization of agrin signaling, its significance in terms of agrin function in CNS neurons is unknown. Recently, evidence has been provided that agrin plays an important role in neural differentiation by showing that agrin-deficient neurons exhibit reduced responses to glutamate both in cell culture and in vivo (Hilgenberg et al., 2002). Therefore, to learn more about the role of the 20-kD COOH-terminal region in agrin function, the ability of the C-Ag20 isoforms to rescue the agrin deficiency was tested.

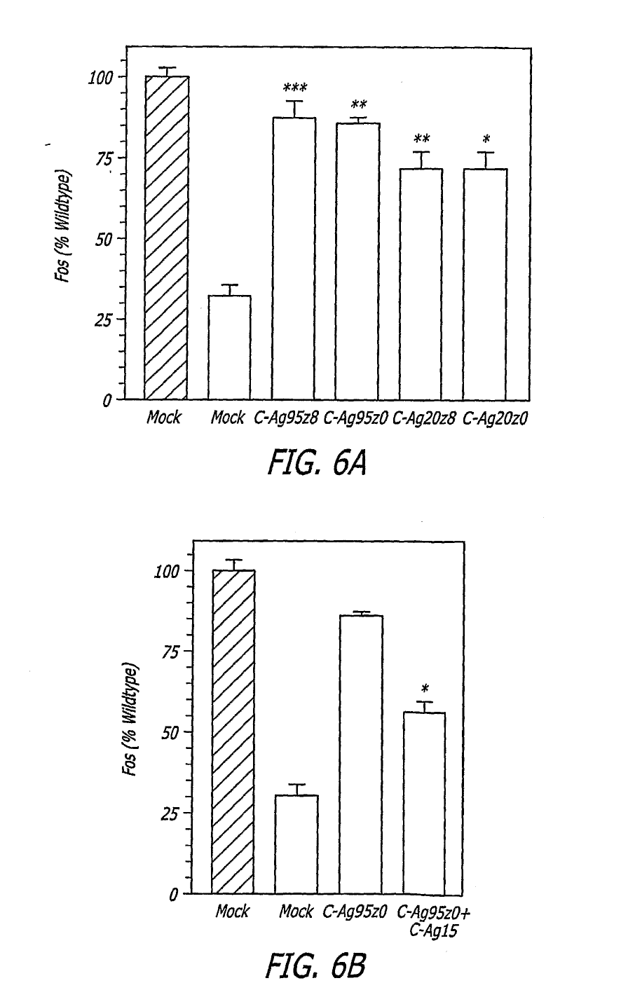

When grown in normal media, levels of glutamate-induced Fos expression in agrin-deficient neurons were reduced by ˜70% compared with wild type. However, consistent with these earlier findings, supplementing the growth media for 2 d with a saturating concentration of either C-Ag95_{z8} or C-Ag95_{z0} significantly increased the response of agrin-deficient neurons close to wild-type levels (FIG. 6A). Similar results were obtained when agrin-deficient neurons were grown in the presence of the C-Ag20_{z0/8} fragments. Compared with agrin-deficient neurons in vehicle-supplemented control media, treatment with either 5 nM C-Ag20_{z8} or C-Ag20_{z0} resulted in a significant 2.2-fold increase in glutamate response. Thus, not only do the C-Ag20_{z0/8} isoforms exhibit full activity in terms of their ability to induce c-fos, they are also competent to reverse the physiological deficit resulting from loss of expression of full length agrin in the agrin-deficient cultures.

Despite its apparent effectiveness as an antagonist of agrin-induced expression of c-fos, growth in media supplemented with up to 10 nM C-Ag15 had no effect on glutamate-induced expression of c-fos in either wild-type or heterozygous agrin-deficient neurons (unpublished data). This suggests that C-Ag15 is unable to block signaling by native agrin. However, the concentration of endogenous agrin in the cultures is unknown, and may exceed the maximal concentration of C-Ag15, especially if agrin is present as high density clusters on neuronal surface membranes (see below). As an alternative approach to examining the functional properties of the C-Ag15 fragment, its ability to inhibit rescue of the agrin-deficient phenotype by C-Ag95_{z0/8} was tested. Inclusion of 10 nM C-Ag15 in the growth medium significantly reduced the efficacy of 5 nM C-Ag95_{z0}, blocking slightly more than half (54%) of the rescue normally achieved by C-Ag95_{z0} alone (FIG. 6B). Similar results were also seen in single experiments using either C-Ag95_{z8} or C-Ag20_{z8} (unpublished data). Based on these observations it is concluded that, under the appropriate conditions, C-Ag15 can inhibit agrin function in CNS neurons.

### Example 5

### Agrin Receptors are Concentrated at Neuron-Neuron Synapses

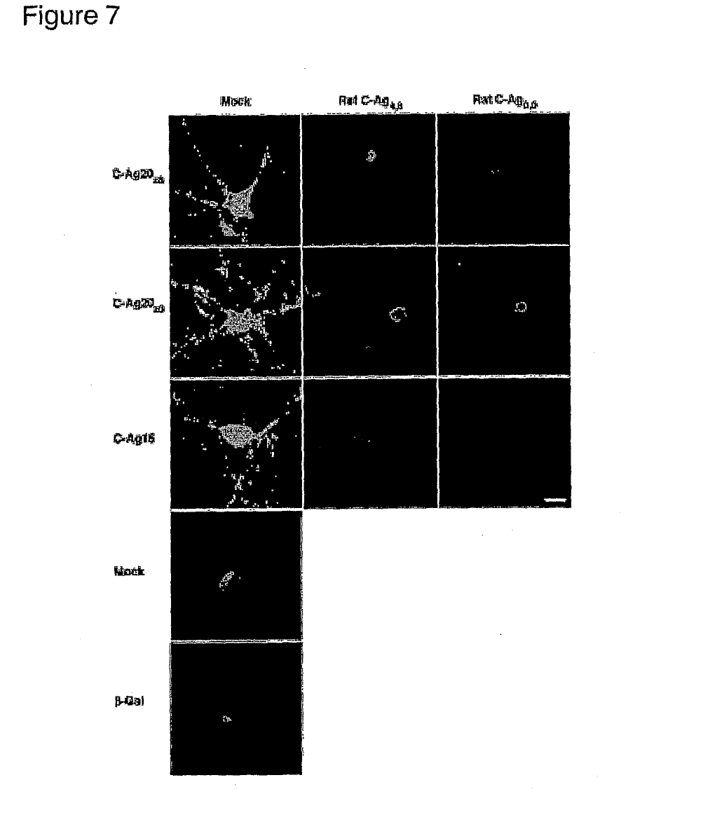

The works described so far provide evidence for a neuron specific agrin receptor, and identify a minimal fragment of agrin capable of activating it. Next, the cellular distribution of the neuronal receptor for agrin using the short agrin fragments as affinity ligands was investigated. Live cortical cultures were incubated in C-Ag20_{z8}, C-Ag20_{z0}, or C-Ag15, and were then labeled with an antibody against the polyhistidine epitope tag to visualize bound agrin. A similar pattern of labeling was evident for all three agrin fragments and appeared as bright puncta scattered over neuronal somata and neurites (FIG. 7). Labeling was specific in that normeuronal cells were not labeled (unpublished data), and little or no labeling was evident when either the agrin fragments were omitted or a β-galactosidase-myc-6His vector control protein was used in their place (FIG. 7). More significantly, binding of all three agrin fragments was completely blocked in cultures labeled in the presence of a large excess of rC-Ag with both “neural” rC-Agy4_{z8} and “inactive” rC-Ag_{y0z0} appearing equally effective at blocking binding of either C-Ag20_{z} isoform or C-Ag15. Precise estimates of the concentration dependence of the competition between rC-Ag and the short mouse agrin fragments are difficult to obtain using immunofluorescence. However, NIH Image analysis of the mean pixel intensities obtained from fixed exposure photomicrographs of random fields revealed labeling to be reduced by ±30% in cultures coincubated with a 1:1 molar ratio of C-Ag20_{z8} to rC-Ag_{y4z8}, and barely detectable at 1:100 (unpublished data). Together, these results provided strong evidence that the bioactivity of the different agrin isoforms shown herein is mediated through binding to a single population of agrin receptors expressed on cortical neuron cell membranes.

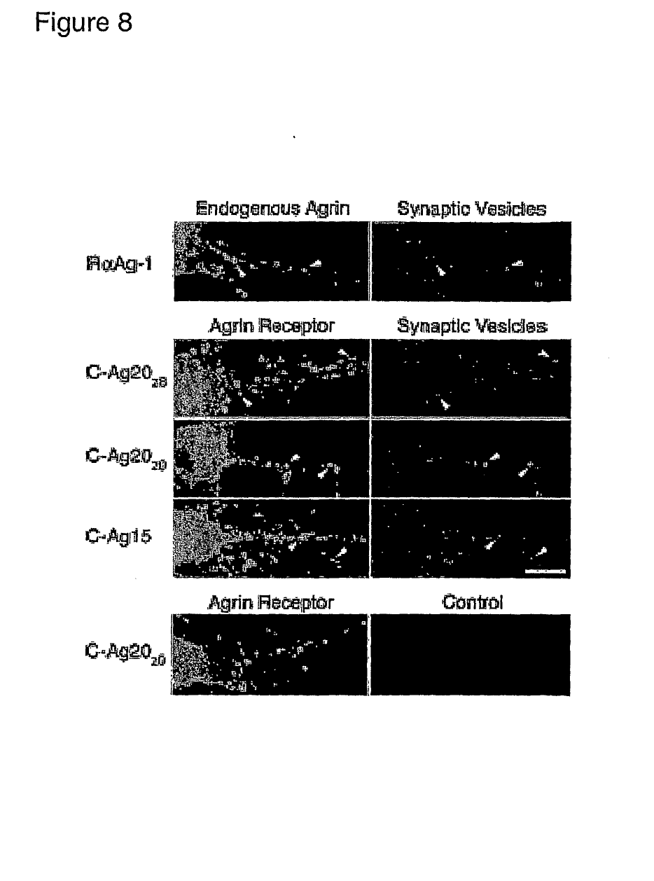

Previous papers have shown that agrin is present at neuron-neuron synapses in peripheral ganglia and retina (Mann and Kroger, 1996; Koulen et al., 1999; Gingras and Ferns, 2001). The punctate labeling observed with the short COOH-terminal agrin fragments suggested that receptors for agrin might also be synaptic. To test this hypothesis, the distribution of endogenous agrin on cultured cortical neurons was first determined by labeling live cultures with RαAg-1, a pan-specific anti-agrin serum, followed by fixation and labeling with a second antibody against the synaptic vesicle protein synaptophysin. Agrin immunostaining was distributed in patches on the neuronal cell bodies and neurites, and colocalized with synaptophysin-positive nerve terminals (FIG. 8). Although some variation in the intensity of the immunostaining was evident, few (if any) agrin-positive/synaptophysin-negative or synaptophysin-positive/agrin-negative patches were observed. Similar results were also obtained using an anti-agrin mAb (MAB5204; CHEMICON International, Inc.) and rabbit anti-synaptophysin antiserum (unpublished data). Therefore, agrin is specifically localized at synapses formed between cultured cortical neurons.

Next, to learn whether agrin receptors also exhibit a synaptic pattern of expression, a parallel work was performed in which live cortical cultures were labeled with C-Ag20_{z8}, C-Ag20_{z0}, or C-Ag15 as before, followed by fixation and staining for synaptophysin. Irrespective of the agrin probe used, agrin receptor clusters exhibited a high degree of colocalization with the synaptic vesicle marker. Staining for synaptophysin was specific; nonneuronal cells were not labeled by the synaptophysin antibody (unpublished data), and nerve terminal staining was not observed in cultures treated with C-Ag20_{z0} or other short fragments in the absence of the anti-synaptophysin antibody (FIG. 8). Together with the agrin immunostaining, the results of these studies show that both agrin and its receptor are concentrated at neuron-neuron synapses.

### Example 6

### α3-Na⁺/K⁺-ATPase as a Neuronal Receptor for Agrin

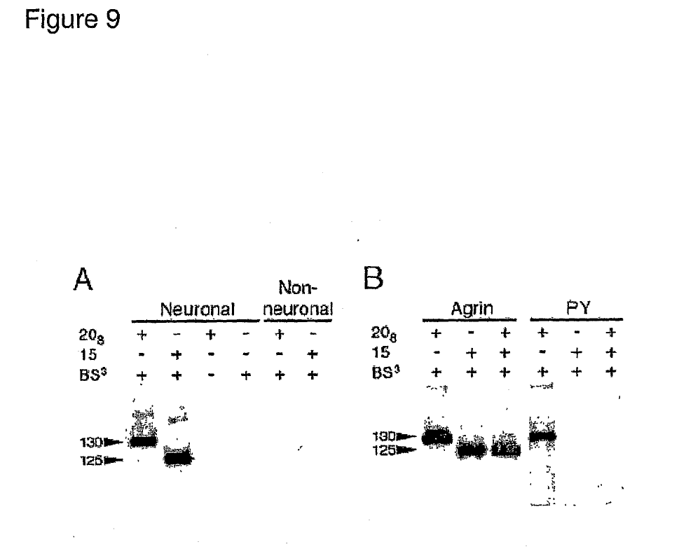

As discussed above, biochemical and Ca²⁺ imaging studies have provided evidence for an agrin receptor in cortical and other central nervous system neurons, distinct from the MuSK/MASC receptor complex responsible for agrin signaling in skeletal muscle (Hilgenberg, et. al., (1999); Hilgenberg et. al., (2004)). Recently, a minimal COON-terminal region of agrin, C-Ag20, has been identified that is sufficient to activate the neuronal receptor; a smaller fragment, C-Ag15, was shown to act as an agrin antagonist (incorporated herein by reference is U.S. Ser. No. 11/011,978). As a first step towards identifying the binding site responsible for agrin activity in neurons, the membrane impermeant bifunctional reagent bis[sulfosuccinimidyl] suberate (BS³; Pierce) was used to chemically cross-link agrin fragments to components present on the surface of cultured cortical neurons and non-neuronal cells. Cell membranes were then isolated and analyzed by immunoblotting with an agrin antiserum, RAg1. Cross-linking C-Ag20₈, or C-Ag15, to neurons resulted in the appearance of clear RAg1 immunoreactive bands with apparent molecular weights of ˜130 kDa and ˜125 kDa, respectively (FIG. 9a). Agrin neither binds to nor activates non-neuronal cells and, consistent with this observation, no specific labeling with RAg1 was observed in blots of non-neuronal cells (FIG. 9a). Similar results were obtained with a second cross-linking agent, dimethyl adipimidate (DMA; Pierce), whose reactive groups are more closely spaced than BS³ (8.6 Å versus 11.4 Å; data not shown). Taking into account the mass of the agrin fragments and assuming a 1:1 stoichiometry, the results suggest that agrin associates with a single class of sites, with an apparent molecular weight of ˜110 kDa present on neuronal cell membranes.

Ligand induced phosphorylation is a common first step in membrane receptor activation and inhibition of tyrosine kinase activity blocks agrin signalling in central nervous system neurons. To determine if agrin induced phosphorylation of the putative agrin receptor, membranes from neurons cross-linked to C-Ag20₈ or C-Ag15, alone or in combination, were dissolved, in a detergent-containing buffer and aliquots immunoprecipitated with RAg1 or the phosphotyrosine antibody mAb4G10 (Upstate). Immunoprecipitated proteins were analyzed by immunoblotting with a monoclonal antibody (9E10.2, Invitrogen) against the COON-terminal myc tag on the agrin fragments. In line with initial results, RAg1 immunoprecipitates probed for myc tagged agrin revealed two adducts of the expected molecular weight (FIG. 9b), but only the 130 kD band cross-linked to C-Ag20₈ was phosphorylated. Even at a 10-fold higher concentration, C-Ag15 did not induce phosphorylation of the cross-linked complex; it was, nonetheless, an effective inhibitor of C-Ag20₈, blocking both binding and phosphorylation by the larger agrin fragment, consistent with C-Ag15's ability to antagonize agrin signalling.

The properties of the agrin adducts suggest that they represent a complex of agrin fragments and a receptor mediates responses to agrin in central nervous system neurons (Hilgenberg, et. al., (2002)). To determine the molecular identity of this putative agrin receptor, C-Ag20₈ adducts, cross-linked with either BS³ or DMA, were affinity purified and the identity of the component proteins determined by mass spectrometry of their tryptic digests (Proteomic Research Services, Inc.). In addition to the expected peptide sequences for agrin, four to twelve peptides were present in each sample that matched the α3 subunit of the Na⁺/K⁺-ATPase. Similar results were also obtained when C-Ag90₈ (R&D Systems), a larger COOH-terminal fragment more commonly used in studies of agrin function, was used in place of C-Ag20₈. Combined, the data for the three samples represented 17% coverage of the α3-subunit amino-acid sequence overall.

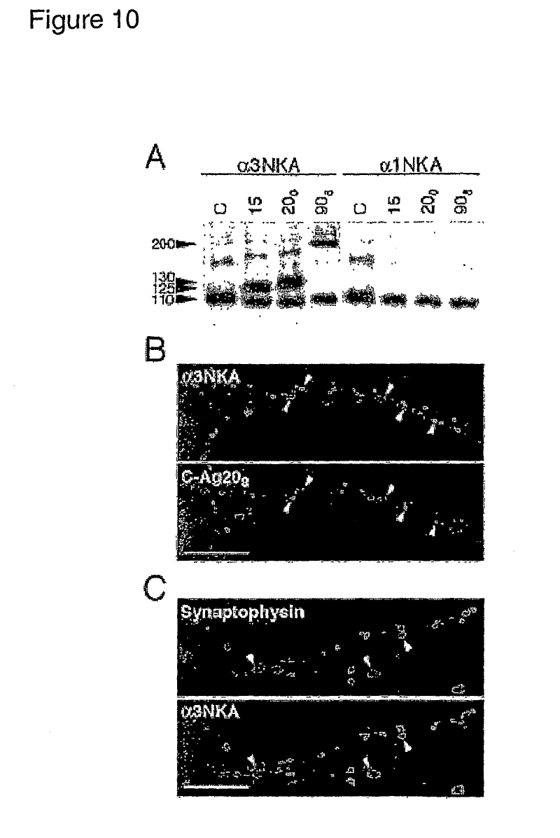

To confirm the results of the mass spectrometry the ability of different α-subunit antibodies to recognize the putative agrin-Na⁺/K⁺-ATPase complex was tested. When probed with the α3 mAb XVIF9-G10 (Novus Biologicals), immunoblots of cultured cortical neurons treated with BS³ alone contained a major 110 kDa band corresponding to the α3 subunit (FIG. 10a). Cross-linking in the presence of C-Ag15, C-Ag20₀, or C-Ag90₈ resulted in a3 positive bands at 125, 130 and 200 kDa, respectively, consistent with agrin binding to the α3-subunit of the Na⁺/K⁺-ATPase. Agrin binding is specific for the α3-subunit, since no molecular weight shift was apparent when the same cell extracts were probed with an antibody to the closely related α1Na⁺/K⁺-ATPase (FIG. 10a).

Previous studies have shown that the α3Na⁺/K⁺-ATPase is distributed in a nonuniform fashion over the soma and processes of cultured hippocampal neurons (Juhaszova, M. et. al., (1997)), reminiscent of the pattern of labelling observed using short agrin fragments as histochemical probes for agrin receptors on cultured cortical neurons. Double labelling with mAb XVIF9-G10 and C-Ag20₈ revealed extensive overlap between the α3Na⁺/K⁺-ATPase and agrin binding sites on cultured cortical neurons (FIG. 10b). Consistent with earlier studies (Hoover et al., 2003), double labeling for synaptophysin and the α3Na+/K+-ATPase shows agrin receptors diffusely distributed over the neuronal soma but concentrated at synapses (FIG. 10c). This observation, together with the results of the biochemical studies, is strong evidence the α3Na⁺/K⁺-ATPase is the neuronal receptor for agrin.

### Example 7

### Agrin Inhibits Activity of the α3Na+/K+-ATPase

Na⁺/K⁺-ATPases are heteromeric proteins composed of α, β, and γ subunits. Multiple isoforms of each subunit are encoded by different genes that exhibit cell-specific patterns of expression. Expression of the α3 subunit in the central nervous system is neuron-specific. Neurons, but not non-neuronal cells, respond to treatment with agrin, suggesting a role for agrin in modulating the function of α3 subunit-containing Na⁺/K⁺ pumps. To test this hypothesis, Na⁺ imaging was used to determine the effect of agrin on cytoplasmic Na⁺ levels in cultured cortical cells.

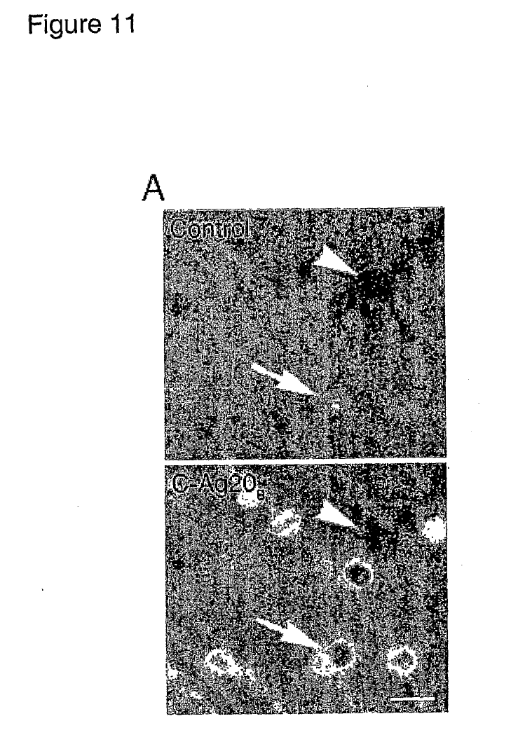

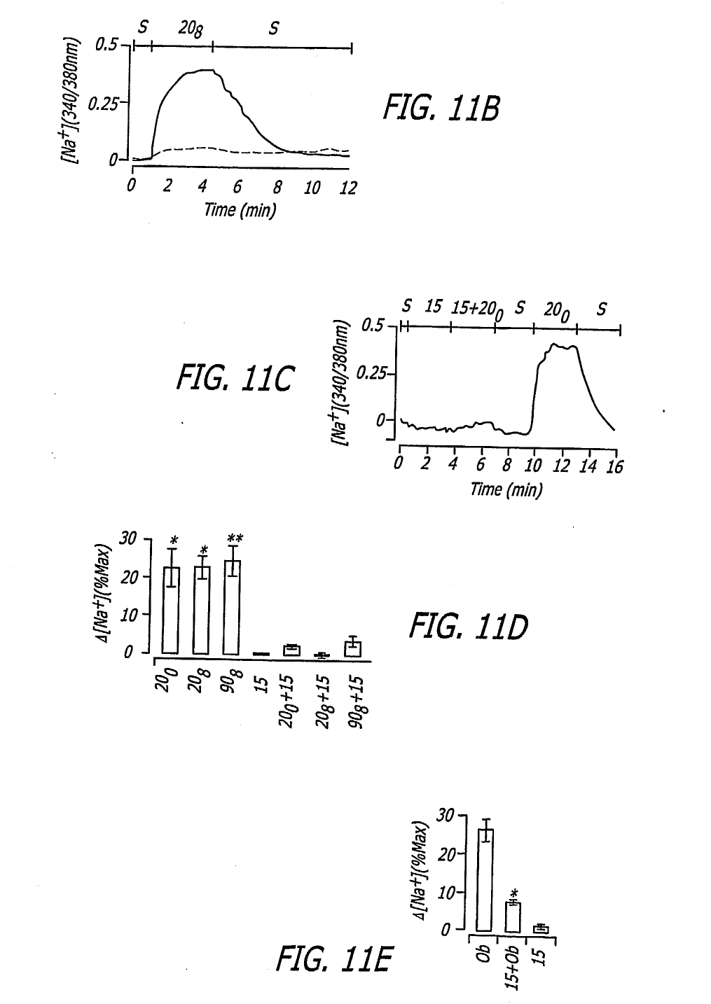

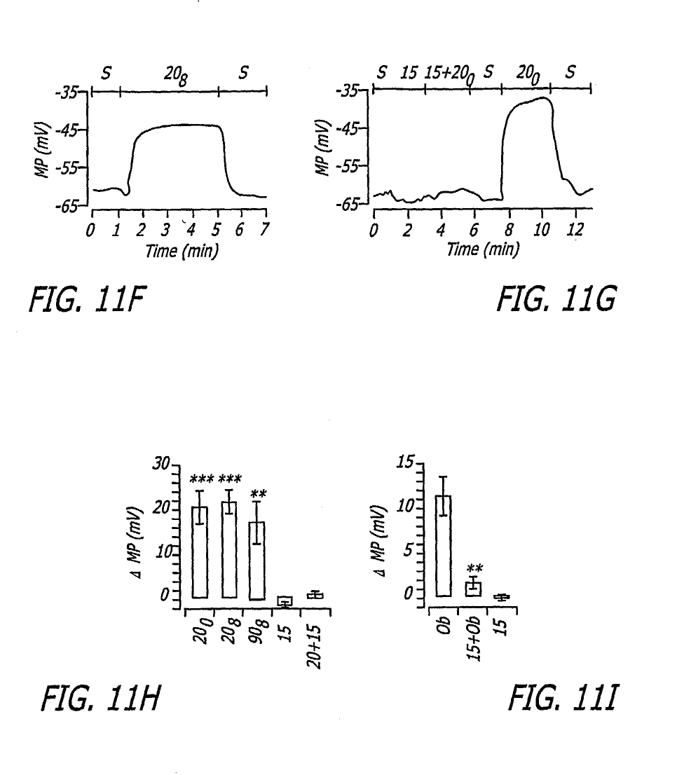

Treatment with C-Ag20₈ caused a rapid increase in neuronal cytoplasmic Na⁺. The response to agrin was reversible and cell specific in that non-neuronal cells were unaffected (FIG. 11a, b). Consistent with its ability to bind but not activate the receptor, C-Ag15 alone had no significant effect on resting Na⁺ levels but blocked the increase induced by the larger agrin fragments (FIG. 11c). Quantitative comparison of the effects of different agrin fragments showed that treatment with either of the alternatively spliced C-Ag20 isoforms or C-Ag90₈ resulted in a significant increase (p<0.01, p<0.05 respectively, Student's t-test) in neuronal intracellular Na⁺ (FIG. 11d). In contrast, C-Ag15 alone had no effect on resting Na⁺ levels but blocked the increase normally induced by each of the active agrin fragments (C-Ag20₀, p<0.05; C-Ag20₈, C-Ag90₈, p<0.01, Student's t-test). Cardiac glycosides, such as ouabain, are specific inhibitors of Na⁺/K⁺-ATPases; the short latency of the agrin response (t_{1/2} to peak=19.7±2.4 s) suggests that agrin might be inhibiting the α3Na⁺/K⁺-ATPase in a similar manner. Consistent with this hypothesis, treatment with a low concentration of ouabain to inhibit the high affinity α3Na⁺/K⁺-ATPase results in an increase in neuronal Na⁺ levels similar to the active agrin fragments (FIG. 11e). Co-incubation with C-Ag15, at the same concentration that blocked the active agrin fragments, blocked the ouabain-induced increase in neuronal Na⁺ (p<0.05, Student's t-test). Based on the ability of C-Ag15 to protect against inhibition of the α3Na⁺/K⁺-ATPase by ouabain, it can be concluded that agrin-induced changes in intracellular Na⁺ levels are mediated directly by inhibition of the α3Na⁺/K⁺ pump.

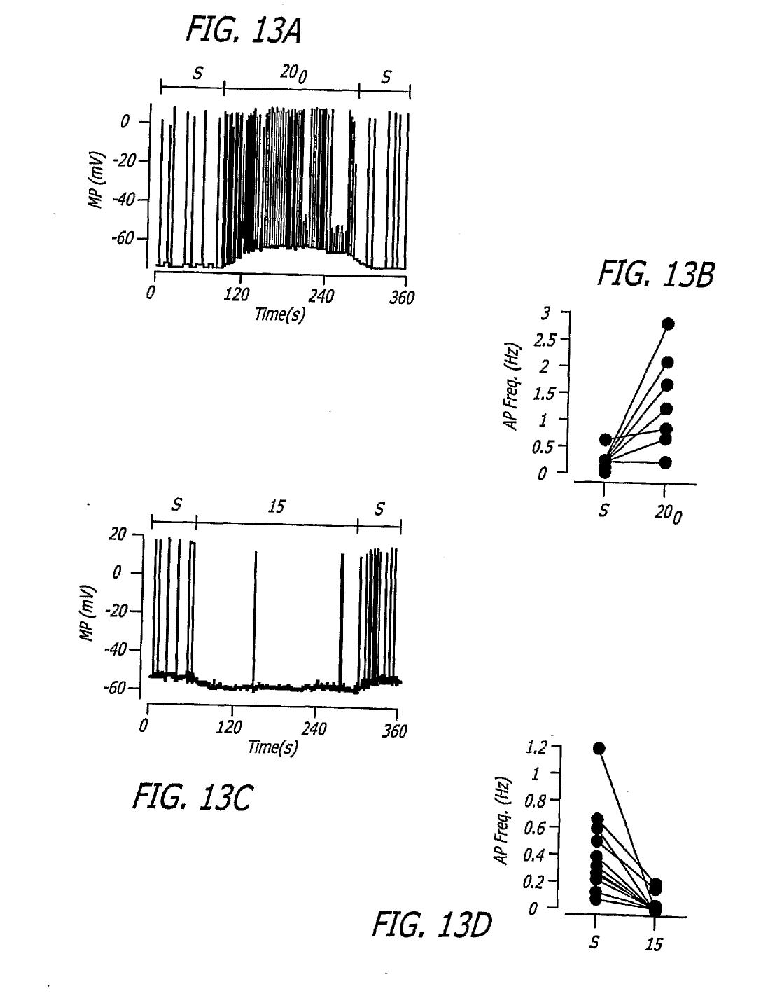

The Na⁺/K⁺-ATPase expels three intracellular Na⁺ ions for every two K⁺ ions taken up, directly affecting the membrane potential of all animal cells. In line with the results of the Na⁺ imaging, whole cell current clamp measurements showed that agrin treatment causes a rapid and reversible depolarization of cultured cortical neurons (FIGS. 13f, h; C-Ag20₀, C-Ag20₈, p<0.001, C-Ag90₈, p<0.01, paired Student's t-test). Moreover, C-Ag15 was also effective in blocking depolarization induced by the active agrin fragments (FIGS. 13g, h; C-Ag20₀, p<0.05; C-Ag20₈, p<0.01, paired Student's t-test). Treatment with C-Ag15 resulted in a small hyperpolarization (−1.0±0.4 mV; p<0.05, paired Student's t-test; FIG. 11g, h), suggesting displacement of endogenous agrin. Consistent with the results of the Na⁺ imaging, ouabain induced membrane depolarization was blocked by C-Ag15 (FIG. 11i; p<0.01, paired Student's t-test), confirmation that agrin's effect on neuron membrane potential is mediated by its interaction with the α3Na⁺/K⁺ pump.

Na⁺/K⁺-ATPases are responsible for maintaining the sodium/potassium ion gradient reflected in the resting membrane potential and necessary for a variety of secondary cellular processes. Thus, many of agrin's effects on neurons are likely to stem directly from its ability to inhibit the α3Na⁺/K⁺-ATPase, resulting in decay of the sodium/potassium ion gradient and membrane depolarization. Particularly noteworthy is that activity of the Na⁺/Ca²⁺ exchanger (NCX) is largely governed by the α3Na⁺/K⁺-ATPase in neurons (Blaustein, et. al., (2002)). The NCX is present on synaptic terminals (Juhaszova, M., et. al., (2000)) and has been shown to regulate neurotransmitter release and synaptic vesicle recycling (Reuter, H. & Porzig, H., (1995); Bouron, A. & Reuter, H., (1996)). The observation that agrin and its receptor, the α3Na⁺/K⁺-ATPase, are also concentrated at synapses, suggests that agrin mediated modulation of synaptic Ca²⁺ levels will have profound effects on synaptic transmission and neuronal excitability. Consistent with this hypothesis, suppression of agrin expression is associated with a decrease in synaptic vesicle cycling in hippocampal neurons (Böse, C. M. et al., (2000)).

### Example 8

### Expression of the α3 Subunit of the Na+/K+-ATPASE is Sufficient for Agrin Binding and Agrin-Evoked Responses

Studies of MuSK, the receptor in skeletal muscle responsible for agrin-induced clustering of AChR, have shown that agrin-MuSK interaction requires a yet to be identified accessory component expressed only in muscle called MASC (Glass et al., 1996). To learn whether agrin interaction with the α3Na+/K+-ATPase might exhibit a similar dependency on cell-context the properties of non-neuronal cells transiently transfected with pRca3, a plasmid expressing the rat α3 subunit under control of the CMV promoter were examined.

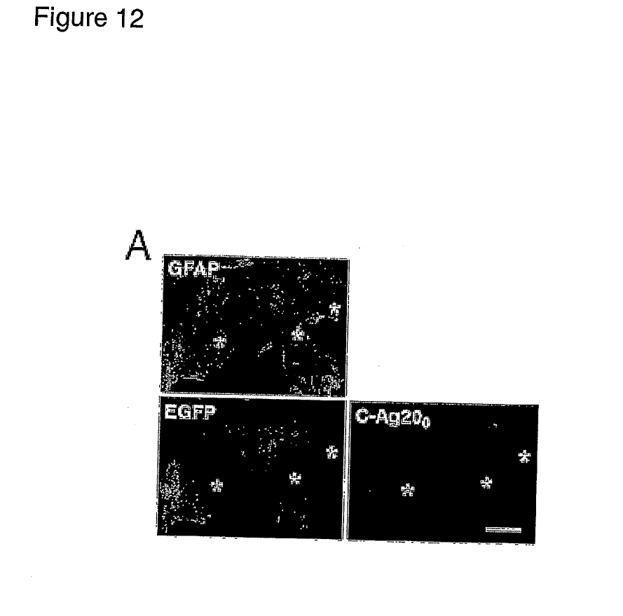

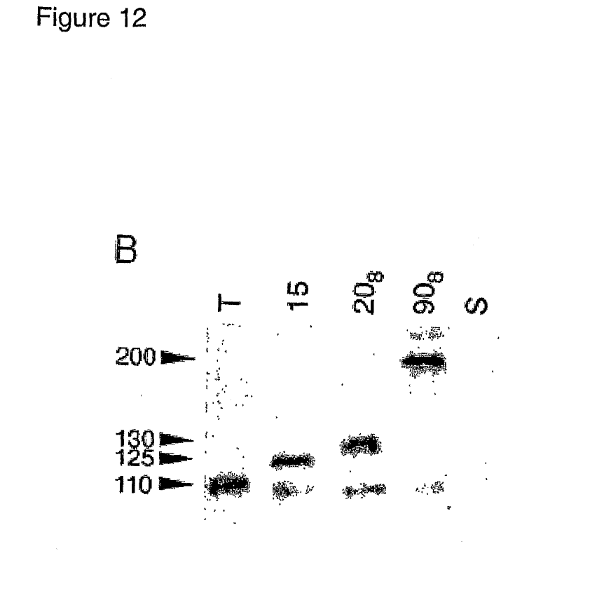

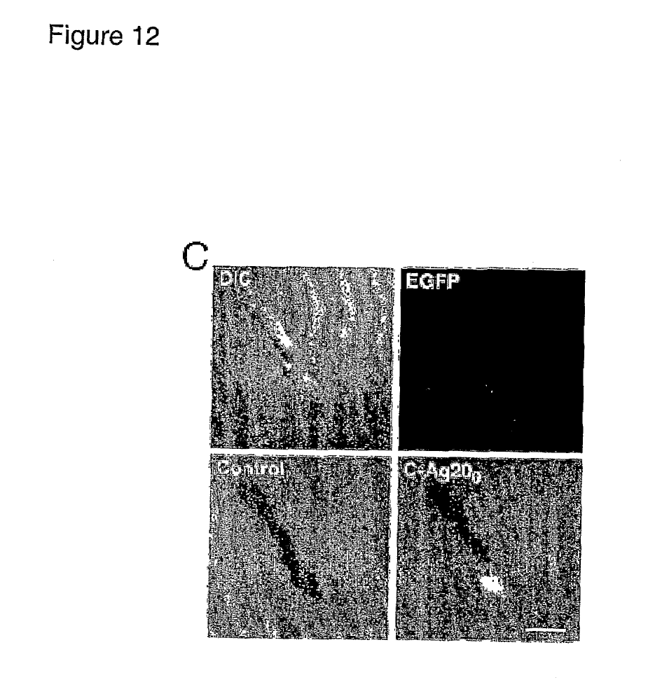

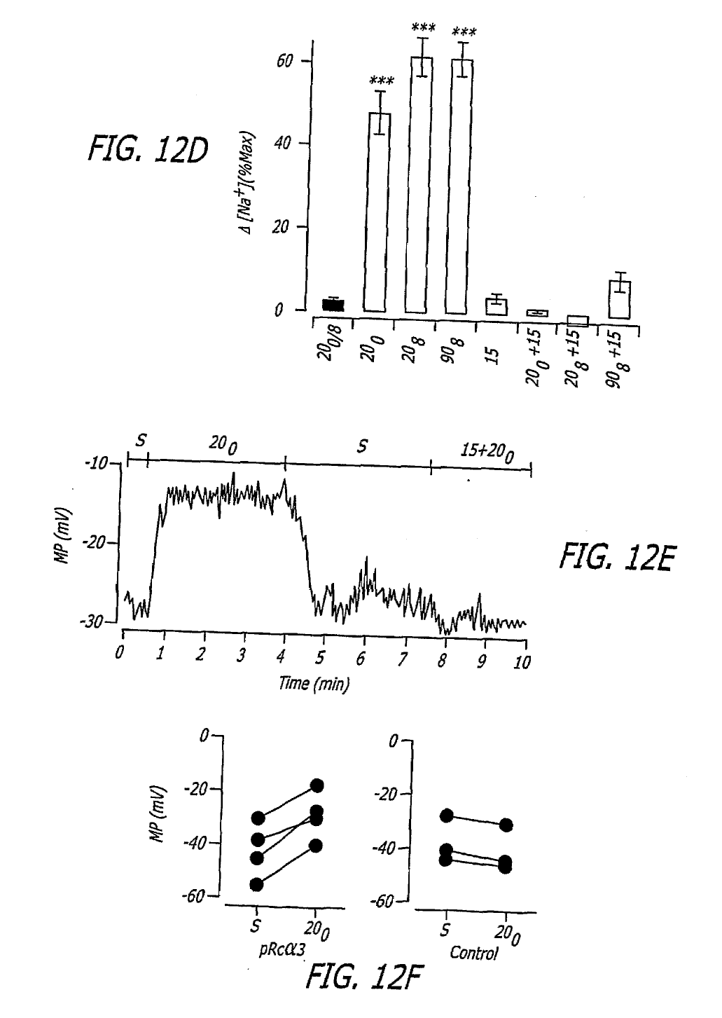

Immunostaining with an antibody to the α3Na+/K+-ATPase showed that non-neuronal cells transfected with pRcα3, but not cells transfected with the enhanced green fluorescent protein marker plasmid pEGFP-C1 alone, expressed the α3 subunit (data not shown). In line with these findings, agrin binding was only observed on the surface membranes of non-neuronal cells expressing pRcα3 (FIG. 12a). Immunoblots of agrin fragments cross-linked to non-neuronal cells confirmed the interaction with the α3 subunit expressed from the transfected plasmid (FIG. 12b). Thus, expression of the α3 subunit of the Na+/K+-ATPASE is sufficient for agrin binding and independent of cell context.

Next the physiological responses of non-neuronal cells transfected with pRcα3 to agrin were examined. Treatment of non-neuronal cells expressing the α3 subunit with either C-Ag20 isoform or C-Ag908 triggered a rapid increase in the concentration of intracellular Na+ ions that was not evident in normal cells or cells transfected with pEGFP-C1 alone (FIG. 12c, d). Consistent with earlier results, Na+ levels were unaffected by treatment with C-Ag15 alone although C-Ag15 proved to be an effective antagonist for the active agrin fragments (FIG. 12d).

Parallel observations were obtained when whole cell current clamp measurements were made to examine the effects of agrin on the electrophysiological properties of non-neuronal cells expressing the α3 construct (FIG. 12e, f). The mean resting potential of non-neuronal cells was higher and more variable than in neurons. Nevertheless, non-neuronal cells expressing the rat α3Na+/K+-ATPase subunit were reversibly depolarized (14.1±1.7 mV) by treatment with either of the C-Ag20 fragments. In contrast, no change in the membrane potential was evident upon agrin treatment of non-transfected non-neuronal cells or cells transfected with pEGFP-C1 alone (FIG. 120, indicating that the response to agrin was specific for the pRcα3 construct. Taken together, these findings provide strong evidence that the α3 subunit of the NA+/K+-ATPASE is the receptor responsible for agrin's effects in cortical neurons.

### Example 9

### Agrin-α3Na+/K+-ATPase Interactions Regulate Neuronal Activity In Situ

The electrogenic activity of the NA+/K+-ATPASE and its role in maintaining gradients of counter ions necessary for the function of other transport proteins suggest that changes in α3Na+/K+-ATPase activity will have profound effects on neuronal function and excitability. To test this hypothesis, the effects of different agrin fragments on the firing properties of cultured cortical neurons bathed in normal external solution was examined.

In line with earlier observations, neurons were rapidly depolarized by treatment with CAg200. However, in the absence of TTX and neurotransmitter receptor antagonists, the agrin induced depolarization was accompanied by a significant and reversible increase in the frequency of spontaneous action potentials (FIG. 13a, b). Similar results were also obtained when neurons were exposed to C-Ag208. Co-incubation with C-Ag15 blocked both the depolarization and increase in action potential frequency induced by either C-Ag20 isoform (data not shown). The response to exogenously applied agrin indicated that the basal level of activity normally present in cultured neurons might be dependent upon endogenous agrin-α3Na+/K+-ATPase interactions. To address this question, the effects of C-Ag15 on spontaneous action potentials in cultured cortical neurons (FIG. 13c, d) were tested. In contrast to C-Ag20, C-Ag15 inhibited spontaneous activity in cortical neurons. The effect of C-Ag15 was reversible in that the frequency of spontaneous action potentials returned to basal levels upon washing with normal external solution (FIG. 13c).

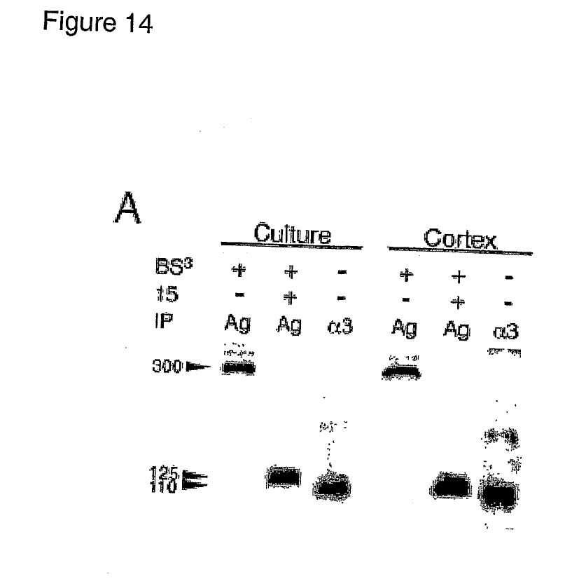

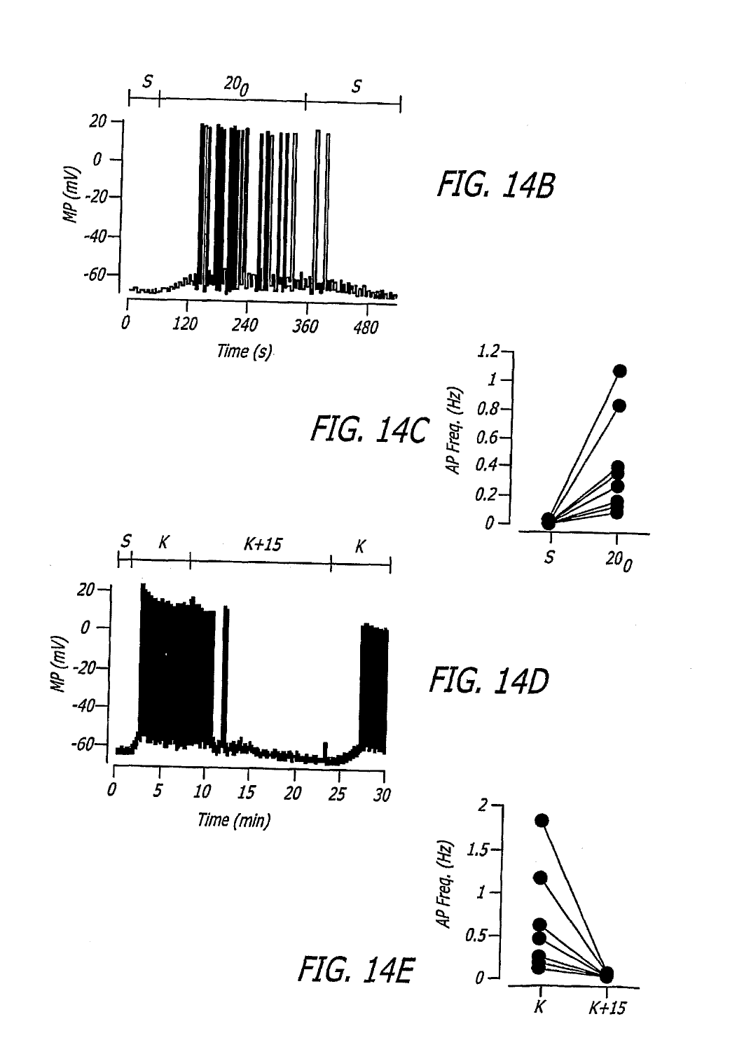

A simple explanation for the effect of C-Ag15 on neuronal activity is that some α3Na+/K+-ATPases are normally inhibited by native agrin; competition by C-Ag15 removes this inhibition, decreasing the probability of firing. Consistent with this hypothesis, pull-down experiments on detergent extracts of cultured neurons cross-linked with BS3 revealed a protein complex with an apparent molecular weight ≧300 kDa recognized by both agrin and α3Na+/K+-ATPase antibodies (FIG. 14a). Moreover, formation of the endogenous agrin-α3Na+/K+-ATPase complex was blocked by cross-linking in the presence of C-Ag15, evidence that endogenous agrin can be displaced from its receptor by C-Ag15. Omission of the cross-linking step in control cultures resulted in the appearance of a 110 kDa band characteristic of the native α3Na+/K+-ATPase. Similar results were obtained when the same experimental paradigm was used to cross-link endogenous agrin-α3Na+/K+-ATPase complexes in cortical slices prepared from 12 day-old mice (FIG. 14a), evidence agrin might be regulating neuronal activity in vivo. To examine this possibility the effects of C-Ag20 and C-Ag15 on the electrophysiological properties of layer V neurons in acute cortical slices prepared from 6-11 day-old mice were tested. Only about 10% of neurons in the slice preparations exhibited spontaneous action potentials. Nevertheless, similar to its effects on cultured neurons, bath application of C-Ag200 resulted in rapid depolarization (Δ9.6±2.22 mV, p<0.001, paired Student's t-test) and appearance of high frequency action potentials (FIG. 14b, c). In line with the results on cultured neurons, the response to C-Ag200 was also blocked by C-Ag15 (data not shown), arguing that the effect of agrin is specifically mediated through inhibition of the α3Na+/K+-ATPase.

The low frequency of spontaneously active neurons in the cortical slices limits the ability to examine the effects of C-Ag15 on ongoing activity, but C-Ag15 reversibly inhibited action potentials in two neurons that were found to be spontaneously active (data not shown). For a more robust test of the role of endogenous agrin in regulating neuronal activity the ability of C-Ag15 to inhibit action potentials induced by the glutamate receptor agonist kainic acid was examined. As expected, treatment with kainic acid (0.5 μM) induced a rapid depolarization accompanied by the appearance of sustained high frequency action potentials in most neurons (FIG. 14d, e). However, within 10 minutes of the addition of C-Ag15, neuron membrane potentials became increasingly more negative to a point where they were comparable to normal resting membrane potentials measured prior to kainic acid/C-Ag15 treatment (−56.2±1.3 mV in kainic acid versus −66.3±1.8 mV in kainic acid+C-Ag15, p<0.01, paired Student's t-test; −70.4±1.6 mV in saline). Action potential frequency also declined over a similar time course, from a mean of 0.6±0.2 Hz in kainic acid to 0.01±0.005 Hz in kainic acid plus CAg15. The fact that C-Ag15's effects on membrane potential and action potential frequency were reversible is evidence of the specificity of C-Ag15 action.

### Example 10

### Parkinson's Disease

Parkinson's disease (PO) is a progressive neurodegenerative disorder that affects approximately 1 million persons in the United States. It is characterized by resting tremor, rigidity, gait disturbance, and postural instability, which result primarily from degeneration of dopaminergic neurons in the substantia nigra pars compacta (SNpc), Although the etiology of PO is incompletely understood, evidence suggests that dopaminergic neurons may be particularly susceptible to oxidative stress and over-production of reactive oxygen species (ROS). Interestingly, SNpc neurons are rich in glutamate receptors that when activated trigger both single and burst firing patterns of action potentials. Over stimulation of excitatory pathways is a common initiator of neurodegeneration. Thus, by virtue of their pattern of neurotransmitter expression and intrinsic membrane properties, SNpc neurons seem predisposed to excitotoxic injury.

The extracellular matrix protein agrin is well known for its role in directing the motor neuron-induced accumulation of acetylcholine receptors at the developing vertebrate neuromuscular junction. Agrin is also expressed in the brain; however, unlike the neuromuscular junction, it is not required for neuron-neuron synapse formation, but appears to play a role in regulating the response of CNS neurons to excitatory stimuli instead. To understand agrin function in the CNS, the signal pathway(s) through which agrin acts have been identified. Initial studies demonstrating that agrin-dependent activation of a receptor on neuron surface membranes triggered a rapid rise in intracellular Ca²⁺ were followed by experiments showing that short fragments of agrin could be used to localize agrin receptors concentrated at neuron-neuron synapses. Using these short agrin fragments as probes, as discussed above, it has been discovered that the neuronal receptor for agrin is the a3 subunit of the Na⁺/K⁺-ATPase. (See FIGS. 11 and 12) Na⁺/K⁺-ATPases play a crucial role in living cells by maintaining ion gradients and resting membrane potential.

Interestingly, expression of the neuron specific α3 subunit has been implicated in regulating the firing pattern of dopamine neurons. Moreover, mutation of the human α3Na⁺/K⁺-ATPase gene has recently been shown to be associated with rapid-onset dystonia Parkinsonism (de Carvalho Aguiar, P. et al., (2004)), an autosomal-dominant movement disorder, suggesting that the agrin/α3Na⁺/K⁺-ATPase pathway regulates neuronal activity. Agrin's interaction with the α3Na⁺/K⁺-ATPase suggests that changes in agrin expression should be correlated with changes in neuronal activity. Consistent with this prediction, heterozygous agrin-deficient mice are less sensitive than their wild type siblings to kainic acid induced seizures. Coupled with the dramatic changes in agrin mRNA levels following seizure (O'Connor, L. T., (1995)), this observation implicates agrin in the etiology of epilepsy. Further, concentration of agrin in amyloid plaques and neurofibrialliary tangles (Verbeek, M. M. et al., (1999)) together with studies showing that inhibition of Na⁺/K⁺ ATPase potentiates α-amyloid induced apoptosis (Xiao, A. Y., (2002)) suggests agrin might also contribute to neuronal dysfunction and cell loss characteristic of Alzheimer's and other neurodegenerative disorders. Dysregulation of intracellular Ca²⁺ is a hallmark of many neurodegenerative disorders and agrin/α3Na⁺/K⁺-ATPase modulation of the plasma membrane NCX is likely to play a critical role in neuronal Ca²⁺ homeostasis. Derivatives of agrin, such as C-Ag15, will be useful starting points for therapeutic agents that might alleviate or reverse the progress of these devastating diseases.

Data from the current invention (see FIGS. 13 and 14) provide strong evidence agrin acts as an endogenous ouabain-like molecule, regulating activity of the α3Na⁺/K⁺-ATPase in neurons. Inhibition of the Na⁺/K⁺-ATPase induces ROS production while agrin-deficient neurons are resistant to excitotoxic injury, suggesting that changes in agrin signaling may contribute to the etiology of idiopathic PD. To test this hypothesis, the effect of agrin on dopamine neuron function and response to excitotoxic injury may be examined in acute brain slice and cell culture. The effects of disrupting agrin signaling on dopamine neurons in vivo may be examined in α3Na⁺/K⁺-ATPase knockout and agrin knockin mice.

Inhibition of the Na⁺/K⁺-ATPase by strophanthidin blocks the NMDA induced burst firing in dopamine neurons (Johnson et al., 1992). Surprisingly, it has been shown that agrin specifically inhibits the α3Na⁺/K⁺-ATPase and provide preliminary evidence this interaction regulates the intrinsic firing rates of cortical neurons (FIGS. 13-16). Therefore, the ability of agrin and its derivatives to modulate firing properties of dompaminergic neurons in acute slices of SNpc are examined in the following examples.

### Example 10a

### Agrin Regulates the Firing Properties of SNpc Neurons (Prophetic)

Preliminary studies show that cultured cortical neurons are depolarized by agrin inhibition of the α3Na⁺/K⁺-ATPase. Others have shown that inhibition of the Na⁺/K⁺-ATPase by strophanthidin blocks NMDA induced burst firing in dopamine neurons. The ability of agrin to modulate firing properties of dopamine neurons in acute slices of SNpc is examined.

Burst firing of action potentials, an important functional adaptation of dopamine neurons, is driven by rhythmic hyperpolarizations generated by the activity of the α3Na⁺/K⁺-ATPase. Understanding mechanisms that regulate dopamine neuron excitability therefore has implications for the development and treatment of PD. Preliminary data show agrin acts as a specific inhibitor of the α3Na⁺/K⁺-ATPase in cortical neurons. Whole cell patch clamp electrophysiology is used to test the ability of agrin to influence the firing properties of SNpc neurons in acute slice preparations.

Horizontal slices of midbrain from adult mice are prepared and whole cell current clamp recordings are made from cells in the SNpc as described (Massengill, J. L., et al., (1997)). Initial studies test the effects of the alternatively spliced minimal active fragments of agrin, C-Ag20₀ and C-Ag20₈, alone or in combination with the shorter agrin antagonist C-Ag15, on the resting potential of SNpc neurons. Experiments are performed in the presence of TTX, CNQX, APV, BMC, dTbC and chlorpromazine, to block voltage and ligand gated channels. Subsequently the effect of the agrin fragments on firing pattern of SNpc in normal ACSF and in ACSF containing 10-301JM NMDA to induce bursting is assessed.

The α3Na⁺/K⁺-ATPase generates a large pump current in midbrain dopamine neurons, suggesting dopamine neurons could be strongly depolarized by treatment with C-Ag20 isoforms. Comparison with results of similar studies on neurons in cortical and hippocampal neurons provides evidence on the relative agrin sensitivity of dopamine neurons. Levels of agrin expression in SNpc and other brain regions is determined by immunohistochemistry and immunoblot, but membrane hyperpolarization following treatment with C-Ag15 is evidence of activation of α3Na⁺/K⁺-ATPase molecules normally inhibited by endogenous agrin.

The study is repeated in the absence of TTX to show whether C-Ag20 treatment alone induces spontaneous action potentials in dopamine neurons. If so, an increase in excitability following a rise in agrin expression in vivo could contribute to excitotoxic injury of dopamine neurons.

NMDA induced burst firing in dopamine neurons is dependent on rhythmic hyperpolarizations generated by the activity of the α3Na⁺/K⁺-ATPase. Preliminary data showing agrin inhibits activity of this pump, suggest burst firing is blocked by either of the C-Ag20 isoforms due to a reduction in ability to hyperpolarize the membrane following a train of action potentials. To learn whether agrin acts upon dopamine neurons directly, experiments are repeated using bathing solutions containing high magnesium/low calcium to block synaptic currents. The ability of exogenous agrin to modulate burst firing is taken as evidence for a role for agrin regulating dopamine neuron excitability. An increase in the frequency of bursts following C-Ag15 treatment provides strong support for this conclusion and suggests possible therapeutic uses of agrin and its derivatives for improving dopamine neuron function.

### Example 10b

### SNpc Neuron Sensitivity to Excitotoxic Injury is Agrin Dependent (Prophetic)

Excitotoxicity has been implicated in the loss of dopamine neurons in PO, but cortical neurons cultured from agrin mutant mice are resistant to excitotoxic injury. To learn whether agrin might regulate vulnerability of dopamine neurons to excitotoxic insult, the effects of agrin and an agrin antagonist on glutamate induced cell death in cultured SNpc neurons is determined.

Ca²⁺ homeostasis is critical for normal neuronal function while uncontrolled increases in intracellular Ca²⁺ result in excitotoxic injury to neurons. Ca²⁺ homeostasis is regulated in part by the plasma membrane Na⁺/Ca²⁺ exchanger (NCX) that is in turn dependent on the electrochemical gradient generated by the Na⁺/K⁺-ATPase. Consistent with this model, agrin mediated inactivation of the pump triggers a rapid increase in intracellular Ca²⁺ while agrin-deficient neurons are resistant to excitotoxic injury, presumably due to the increased activity of the α3Na⁺/K⁺-ATPase in the absence of agrin. The high density of the α3Na⁺/K⁺-ATPase current in dopamine neurons suggests they may be especially sensitive to changes in sodium pump function. Accordingly, the effects of agrin on glutamate excitotoxicity is examined in cultured SNpc neurons.

Dopamine neurons, dissociated from pieces of the SNpc dissected from horizontal slices of the neonatal wildtype, heterozygous, or homozygous agrin knockout mouse midbrain, are grown in cell culture as described (Hilgenberg, L. G. W., et al., (2002)). At 10-14 div, neurons are treated for 5 minutes with different concentrations of glutamate. Four hours after the glutamate treatment, excitotoxic injury to the neurons is assayed by measuring the level of the cytosolic enzyme lactate dehydrogenase (LDH) in the growth medium. To examine the role of agrin in modulating excitotoxic injury, standard curves of glutamate excitotoxicity are compared with plots of LDH release induced by glutamate in the presence of each agrin fragment (C-Ag20₀, C-Ag20₈, C-Ag15). Cell specific differences in response to agrin treatment are assessed by comparison with the results of parallel studies on neurons cultured from other brain regions such as cerebral cortex and hippocampus.

It is shown that the sensitivity of cultured cortical neurons to glutamate excitotoxicity is four-fold higher in wild type versus agrin-mutant neurons. Assuming the high levels of α3Na⁺/K⁺-ATPase expression play a role in protecting dopamine neurons from excitotoxic injury, the difference in sensitivity between agrin^{+/+} and agrin^{-/-} dopamine neurons is proportionately greater that for other types of neurons.

Neuron specific differences in dependence on α3Na⁺/K⁺-ATPase for protection against excitotoxic insult should also be revealed in the response to treatment with different agrin fragments. Agrin appears to inhibit activity of the pump and treatment with either of the active agrin fragments should potentiate excitotoxicity. On the other hand, C-Ag15 is an agrin antagonist, which by blocking the action of endogenous agrin and increasing α3Na⁺/K⁺-ATPase activity, should prove to be neuroprotective.

### Example 10c

### Disruption of Agrin Signaling in Dopamine Neurons In Vivo Results in a Parkinson's-Like Syndrome (Prophetic)

Agrin's role in the function and survival of dopamine neurons may be assessed by the generation and analysis of transgenic mice carrying mutations that alter agrin signaling in dopamine neurons.

By functioning as an endogenous ouabain-like molecule, an agrin mediated decrease in α3Na⁺/K⁺-ATPase activity in dopamine neurons will increase their susceptibility to excitotoxic injury. Thus, knockout of the α3Na⁺/K⁺-ATPase should result in damage or loss of midbrain dopaminergic neurons. Molecular genetic studies of α1- and α2Na⁺/K⁺-ATPase function have been hampered by the fact that mice homozygous for the mutant alleles exhibit an embryonic or perinatal lethal phenotype. Therefore, to ensure the viability of α3Na⁺/K⁺-ATPase mutant mice, the Cre recombinase system is used to generate mice in which loss of target gene function is restricted to dopamine neurons. An ATP1a3^{loxP}/^{loxP} mouse is used for studies of agrin's role in CNS development. To introduce the mutation of ATP1a3 into catacholaminergic neurons, ATP1a3^{loxP}/^{loxP} mice are crossed with Th^{cre}/^{cre} mice (Gelman, D. M., et al., (2003); Lindeberg, J., et al., (2004)), in which expression of Cre recombinase is driven by the promoter for tyrosine hydroxylase. Loss of α3Na⁺/K⁺-ATPase expression in SNpc of ATP1a3^{loxP}/^{loxP} Th^{cre}/^{cre} mice is confirmed by immunocytochemical staining. Initial characterization of the ATP1a3^{loxP}/^{loxP} Th^{cre}/^{cre} phenotype includes behavioral measurement of locomotor responses to systemic administration of amphetamine and nonbiased stereological assessment using the optical dissector method of the number of TH-immunoreactive and Nissl-positive cells in SNpc.

The ability of exogenous agrin to inhibit sodium pump activity suggests only a fraction of the α3Na⁺/K⁺-ATPase molecules in neuron plasma membranes are normally bound by endogenous agrin. Thus, a dopamine neuron specific α3Na⁺/K⁺-ATPase knockout represents an extreme form of functional silencing that could be triggered by agrin. Accordingly, the effects of agrin overexpression is examined in a transgenic mouse line in which agrin is expressed in a neuron-specific and doxycycline-dependent manner. These double-transgenic mice express the tetracycline-sensitive transactivator (Tet-Off system) under the control of the prion protein promoter to regulate expression of agrin fused to a tetracycline response element (TRE; Peters, H. C., et al., 51-60 (2005)). A COOH-terminal polyhistidine tag facilitates monitoring levels of agrin transgene expression by immunohistochemistry and western blot. Expression of the agrin transgene is initiated by withdrawal of doxycyline and animals monitored for changes in locomotor responses and numbers of dopamine neurons in SNpc as in the ATP1a3 knockout mice.

Human ATP1a3 mutants exhibit rapid-onset dystonia Parkinsonism but the cellular mechanism remains unknown. Motor disturbances in ATP1a3^{loxP}/^{loxP} Th^{cre}/^{cre} mice would suggest that ATP1a3 expression in dopaminergic neurons is required for normal locomotor activity. Given this outcome; it will be interesting to learn whether behavioral changes precede overt loss or damage to TH-positive neurons and whether SNpc neurons are affected disproportionately. Electrophysiological studies of slice preparations of ATP1a3⁺/⁺ and ⁻/⁻ midbrains may be performed to validate the role of α3Na⁺/K⁺-ATPase in shaping the firing properties of SNpc neurons.

Where the efficacy of the Cre induced ATP1a3 knockout is confirmed, absence of an observable phenotype is evidence the α3Na⁺/K⁺-ATPase is not required for dopamine neuron function and would suggest further that disturbances of locomotor activity in human ATP1a3 mutants is due to a loss of α3Na⁺/K⁺-ATPase function by neurons outside of the dopamine system. Since the α2Na⁺/K⁺-ATPase serves functions in nonnervous tissue similar to the α3Na⁺/K⁺-ATPase, the possibility that changes in expression of other Na⁺/K⁺-ATPases might compensate for mutation of ATP1a3 should be examined.

The finding that over-expression of agrin alters locomotion or other behavior such as susceptibility to seizures is strong evidence for a role for agrin in regulating neuronal activity in vivo. As with the studies of the ATP1a3 knockouts, neuronal counts should be made to determine if changes in behavior are correlated with changes in SNpc neuron number and whether SNpc neurons exhibit enhanced susceptibility to increased agrin levels. An advantage of the Tet-Off system is that when doxycline treatment is reinstated expression of the agrin transgene should be suppressed, allowing reversibility of the behavioral and cellular changes to be assessed. Since it is not currently known if agrin acts in an autocrine and/or paracrine agent, a system can be chosen that will drive high levels of agrin transgene expression in SNpc neurons as well as the inputs to them. A positive result suggests the generation of new mice in which the effects of agrin expression in more restricted neuronal populations could be examined, for example, by crossing the TRE-agrin mice with mice in which expression of tTA was regulated by the TH promoter.

Accordingly, it has been determined the α3Na⁺/K⁺-ATPase is a neuronal receptor for agrin and further shown that agrin blocks α3Na⁺/K⁺-ATPase function. Excessive activity is toxic to neurons and one hypothesis of PO is that functional adaptations of SNpc neurons make them particularly susceptible to excitotoxic damage. These findings place agrin directly on a pathway that controls neuronal excitability suggesting that agrin might serve as an important regulator of the firing patterns of dopamine neurons and/or their response to excitatory neurotransmitters. Thus, changes in agrin expression could trigger or exacerbate PD, and drugs able to potentiate α3Na⁺/K⁺-ATPase activity are likely to have therapeutic value in the treatment of PD. The agrin antagonist, C-Ag15, appears to be an excellent starting point for the development of such drugs.

### Example 11

### NA+/K+-ATPASE Role in Treating Ion-Pump Disorders

Sodium, potassium ATPases (NA+/K+-ATPASEs) are responsible for generating the electrochemical gradient of sodium and potassium ions required for the function of other transporters and ion channels in animal cell the plasma membranes. NA+/K+-ATPASEs are heterodimers composed of a catalytically active α subunit and smaller β subunit. Four α subunit genes and three β subunit genes have been identified. Whereas all combinations of α and β subunits form functional pumps, tissue specific patterns of subunit gene expression suggest important functional differences between pump isomers. Classical pharmacological inhibitors of the NA+/K+-ATPASE include cardiac glycosides such as ouabain and digitoxin, valued for their inotropic properties in the treatment of congestive heart failure. The current invention has identified an endogenous ligand, agrin, that binds to and inhibits a3 subunit-containing NA+/K+-ATPASEs. Accordingly, short (20 kDa; C-Ag200 and C-Ag208) fragments of agrin have been identified that, like the full-length protein, inhibit a3NA+/K+-ATPASE activity; a shorter (15 kDa; C-Ag15) fragment acts as an agrin antagonist, disrupting endogenous agrin-a3NA+/K+-ATPASE interactions. The ubiquitous expression of NA+/K+-ATPASEs, their fundamental role in intracellular ion homeostasis, and implication in a variety of human diseases suggests a broad therapeutic potential for agrin derivatives and small molecules modeled on agrin-NA+/K+-ATPASE interactions. Specific areas include, but are not limited to treatment of ion pump disorders.

### Example 11a

### Agrin Effect on Excitotoxic Injury

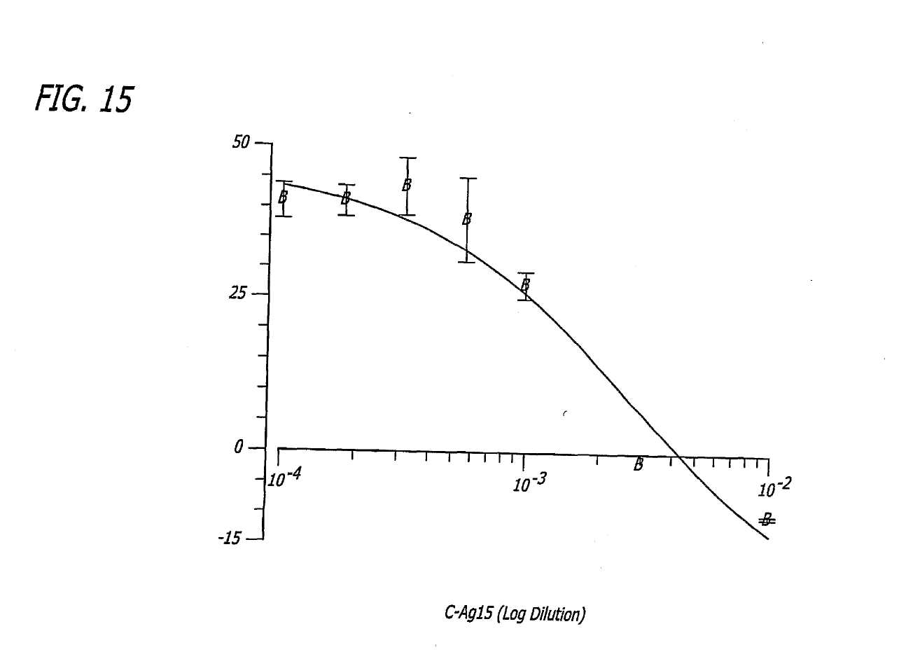

Agrin and the a3NA+/K+-ATPASE are highly expressed in CNS neurons. Treatment with C-Ag20 increases firing frequencies whereas C-Ag15 blocks spontaneous action potentials. Increased or decreased signaling through the agrin-α3Na+/K+-ATPase pathway is likely to be pathological and agrin derivatives will be useful agents for normalizing this activity. The ability of C-Ag15 to block spontaneous neuronal activity has clear applications for treatment of epilepsy. Consistent with this observation, agrin mutant mice are resistant to seizure-inducing stimuli. Preliminary data indicate that C-Ag15 is neuroprotective in an in vitro model of excitotoxic injury (FIG. 15), which is relevant to the treatment of traumatic injury. Consistent with this finding, agrin deficient neurons are less sensitive to excitotoxic injury.

### Example 11b

### Congestive Heart Failure

It has been almost 20 years since immunhistochemical studies (Godfrey et al., 1988), subsequently supported by in situ and PCR analysis (Biroc et al., 1993; Hoch et al., 1993), demonstrated agrin expression in vertebrate cardiac myocytes. Despite the length of time that the scientific community has been aware of this observation, agrin function in the heart has remained a mystery. Much of what is known about agrin function at the neuromuscular junction has come from studies of its interaction with MuSK. In comparison, the lack of knowledge concerning receptors in cardiac muscle that might mediate agrin's effects has been a major stumbling block to understanding agrin function in heart. The recent demonstration that, by regulating the function of the α3Na+/K+-ATPase, agrin modulates excitability of CNS neurons (Hilgenberg et al., 2006) suggests a related role in other excitable tissues where agrin and the α3Na+/K+-ATPase are co-expressed. Here it is demonstrated that agrin, through its interaction with the α3Na+/K+-ATPase, regulates cardiac myocyte function.

According to the American Heart Association nearly 5 million Americans are living with congestive heart failure, with 550,000 new cases diagnosed each year. While not diminishing the impact on quality of life, the estimated direct and indirect costs of treatment and lost productivity currently stand at $29.6 billion, a figure expected to grow as the population ages (Thom et al., 2006). Thus, understanding mechanisms that control cardiac myocyte contraction and characterizing changes in cardiac muscle physiology that accompany normal development and aging are key steps towards identifying genetic and environmental factors responsible for heart disease. The current invention is provides evidence for a previously unsuspected role for agrin and open up a novel line of enquiry into the basic physiology of E-C coupling and regulation of cardiac muscle contraction.

Because of their positive inotropic effects, digitalis and its derivatives are a common component in the armamentarium of therapeutics used to treat heart failure. These drugs, however, are not without their side affects, such as nausea or vomiting, impaired kidney function, headache and other CNS disturbances, related to the fact that cardiac glycosides affect function of all Na+/K+-ATPase isoforms. The current invention provides critical insights into the role of different Na+/K+-ATPases in cardiac myocyte function and the development of drugs for treating congestive heart disease with higher specificity and fewer side effects than those currently in use.

For example, digitalis preparations are often prescribed to increase force of heart contraction and control some forms of atrial fibrillation in patients with congestive heart failure. Presumed targets for digitalis are the α1, α2, and a3NA+/K+-ATPASEs, all of which are expressed in human heart Wang et al., 1996). However, the function of different NA+/K+-ATPASEs is not equivalent. Moreover the therapeutic window for digitalis is very narrow and often accompanied by unwanted behavioral side effects, presumably due to cardiac glycoside mediated inhibition of NA+/K+-ATPASEs on CNS neurons. Thus, there is a need for better-behaved drugs that either do not cross the blood-brain barrier and/or exhibit greater specificity than those currently available.

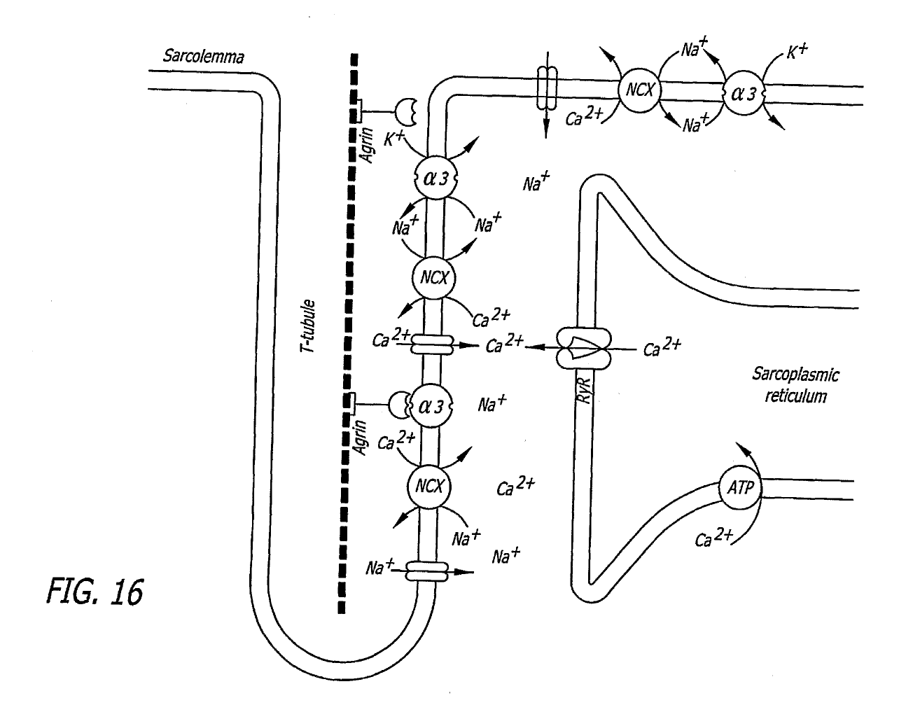

As previously discussed, agrin increases excitability of CNS neurons by specifically inhibiting the function of the α3Na+/K+-ATPase, where the resulting collapse of the Na+ gradient has two main effects. First, depolarization of the plasma membrane increases the open probability of voltage-gated channels, bringing the cell closer to firing threshold and triggering Ca2+ influx through L- and N-type channels driving neurotransmitter release. Second, because of its dependence on the local Na+ gradient generated by the α3Na+/K+-ATPase (Annunziato et al., 2004), Ca2+ efflux through the NCX is slowed or even reversed, potentiating the effects of Ca2+ influx through voltage gated channels. Conversely, neurons become less excitable in the absence of agrin because disinhibition of the α3Na+/K+-ATPase stabilizes the membrane potential and enhances the activity of the NCX (Hilgenberg and Smith, 2004; Hilgenberg et al., 2006). Here a similar model is proposed for agrin regulating cardiac myocyte function (FIG. 16). The data that follows will provide support for this mechanism.

Ca2+ is the primary second messenger responsible for E-C coupling in cardiac muscle. The observation that agrin and the α3Na+/K+-ATPase are co-expressed in cardiac myocytes together with agrin's ability to modulate cytoplasmic Ca2+ levels in neurons suggested a role for agrin-α3Na+/K+-ATPase signaling in regulating cardiac myocyte contractility. To test this hypothesis the effects of mutation of the Agrn gene on cardiac muscle function were examined.

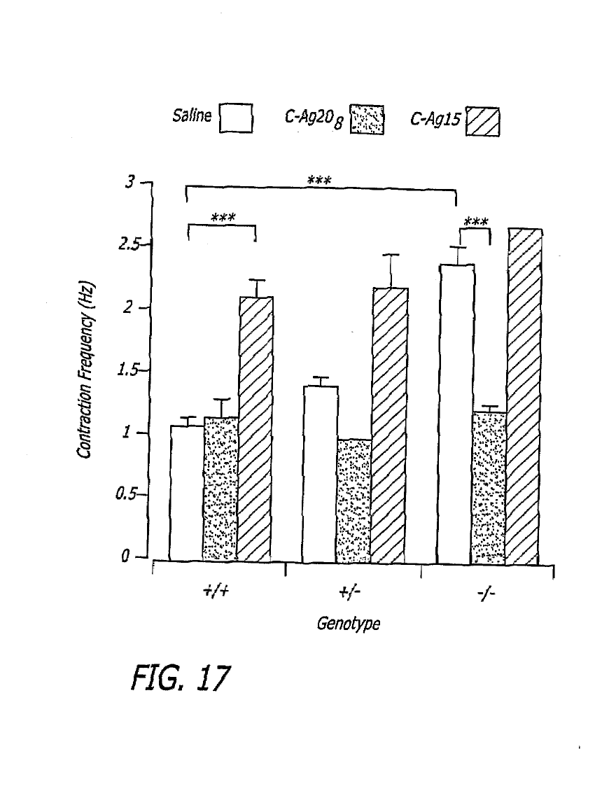

When grown in dissociated cell culture, cardiac myocytes, either in groups or separately, exhibit spontaneous rhythmic contractions. Initial observations suggested that the contraction frequency of myocytes prepared from Agrn−/− embryos is higher than wild type. Quantitative studies confirmed and extended these observations by showing that the frequency of myocyte contraction is dependent on agrin gene dose: Agrn−/− myocyte contraction frequency is about 2-fold higher and Agrn+/− 1.5-fold higher than Agrn+/+ myocytes (FIG. 17). To learn whether the effect of the Agrn mutation on myocyte contraction is a direct result of the loss of agrin expression the effects of growing the cells in media supplemented with C-Ag208, an active agrin fragment, or C-Ag15, the agrin antagonist was next examined.

Growth in the presence of C-Ag208 resulted in a significant decrease in contraction frequency of both Agrn+/− and Agrn−/− myocytes, leading to the conclusion that agrin is required for the development of normal muscle contraction. Interestingly, although growth in C-Ag15 had no effect on Agn−/− cells, it significantly increased the frequency of Agrn+/+ and Agrn+/−myocytes. C-Ag15's ability to phenocopy the Agrn mutation in the wild type cells is strong evidence that endogenous agrin-α3Na+/K+-ATPase signals modulate myocyte contraction. Moreover, the ability of the agrin fragments to modulate the contraction frequency of cultured myocytes demonstrates that the effect of the Agrn mutation is cell autonomous rather than secondary to the loss of agrin function in, for example, autonomic input to the heart.

It may seem counterintuitive to find that Agrn−/− myocytes and myocytes treated with C-Ag15 contract at a higher frequency when the same conditions are associate with a decrease in excitability in neurons. Nevertheless, these observations are consistent with the model. Myocyte contraction frequency is an integral of the velocity of contraction and relaxation; for relaxation, the concentration of cytoplasmic Ca2+ must fall below the threshold for contraction. Removal of Ca2+ takes place by two main routes: sequestration to the SR via the SERCA and efflux through the sarcolemma mediated by NCX1. The current model predicts that suppressing agrin-α3Na+/K+-ATPase interaction, either by knocking out the Agrn gene or treatment with C-Ag15, will enhance the activity of NCX1, thereby decreasing the time to relaxation and increasing the overall frequency of contraction. Effects of agrin on NCX1 mediated Ca2+ clearance are examined as part of Specific Aim 4.

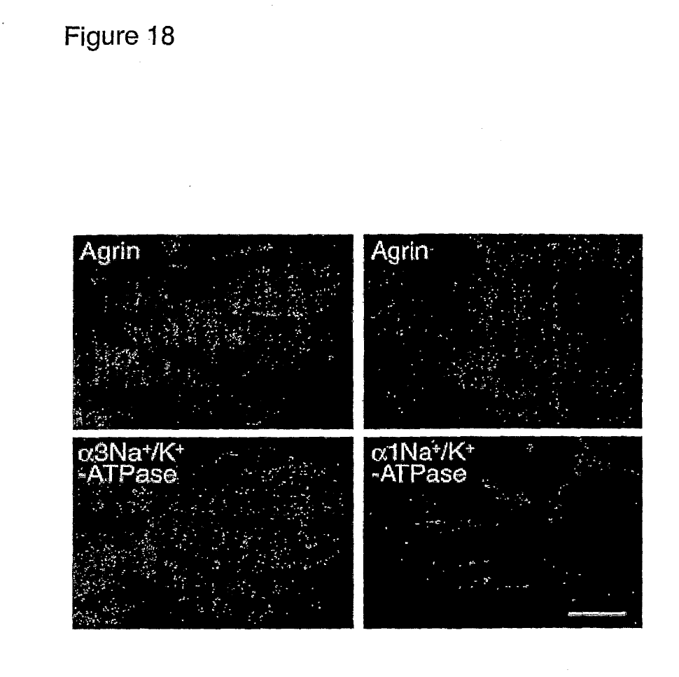

Cardiac myocytes express both agrin and α3Na+/K+-ATPase, but the cellular and subcellular distribution of these two proteins has not been explored. Since agrin action depends on its availability to interact with the α3 subunit of the Na+/K+-ATPase, a double label fluorescence immunohistochemistry can be used to examine the spatial expression of agrin and the α3 subunit of the Na+/K+-ATPase on cultured cardiac myocytes. By 4 days in culture, virtually all myocytes were labeled for both agrin and the α3 subunit. Labeling appeared as fine puncta over surface membranes with a high degree of overlap apparent between the two proteins (FIG. 18). Thus, at the level of resolution afforded by the light microscope, agrin and the α3 subunit of the Na+/K+-ATPase are expressed on cultured cardiac myocytes with a spatial distribution consistent with allowing interaction between them.

Agrin Regulates Intracellular Na+ and Ca2+ Levels in Cardiac Myocytes

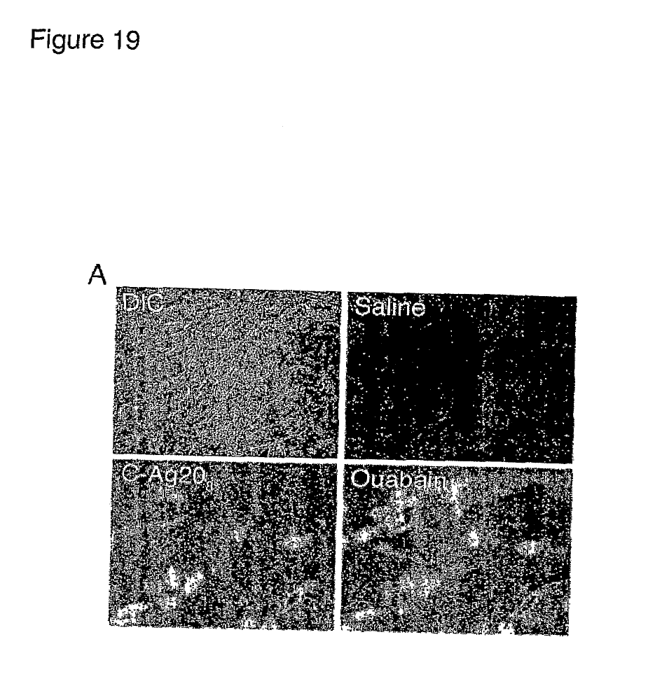

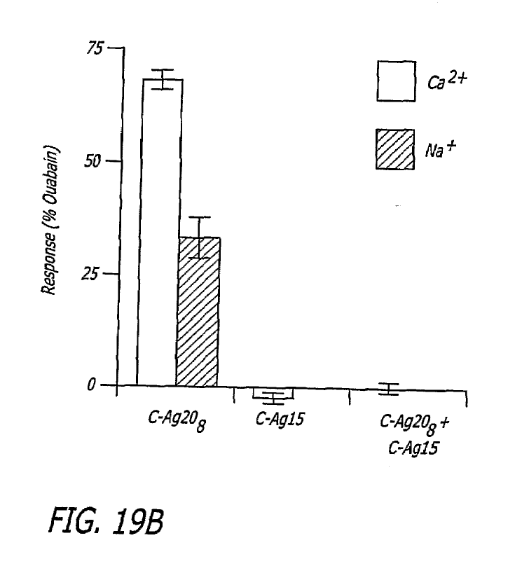

Agrin induced inhibition of the α3Na+/K+-ATPase on CNS neurons leads to a rapid rise in the concentration of cytoplasmic Na₊ and Ca2+ ions (Hilgenberg and Smith, 2004; Hilgenberg et al., 2006). To learn whether a similar agrin signal pathway exists in cardiac muscle, the effects of different agrin fragments on intracellular ion concentrations in cultured cardiac myocytes, loaded with either the Ca2+ sensitive dye Fura-2 or Na+ sensitive dye SBFT, were examined. Consistent with the results in neurons, cardiac myocytes treated with C-Ag208 exhibited a rapid increase in the concentration of intracellular Ca2+ or Na+ ions (FIG. 19). Developmental studies have yet to be performed, but by 4 days in culture the majority of cells (≈90%) of the cells were agrin sensitive. Interestingly, the agrin-induced increase in cytoplasmic Ca2+ was more robust than the Na+ response. While this may be due in part to the higher affinity of Fura-2, influx through voltage-gated Ca2+ channels stimulating Ca2+ release from the SR is also likely to be a contributing factor. Consistent with the current model of agrin-α3Na+/K+-ATPase interaction, Ca2+ imaging experiments show that C-Ag15 is an effective agrin antagonist (FIG. 19B).

Taken together these data provide strong support for a role for agrin in Na+ and Ca2+ homeostasis in cardiac myocytes. Studies in the present application will extend these observations by completing the analysis of the effects of different agrin fragments on the dynamics of intracellular Na+ and Ca2+ and identification of the sources and sinks involved.

Agrin Inhibits a Ouabain-Resistant Mutant α3Na+/K+-ATPase

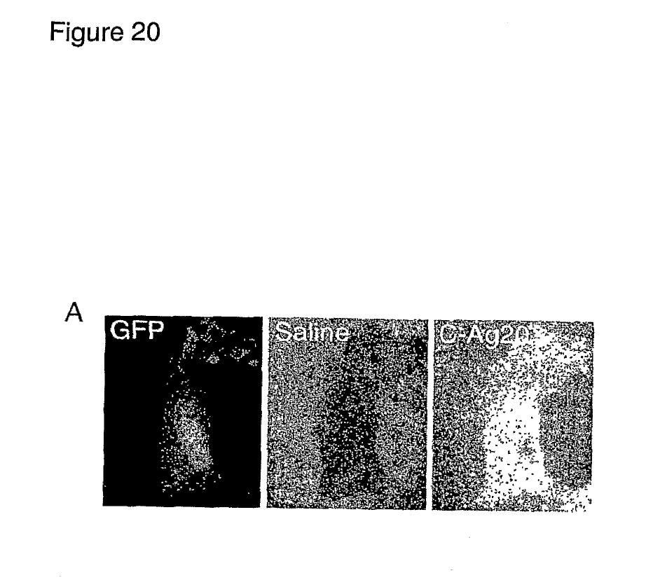

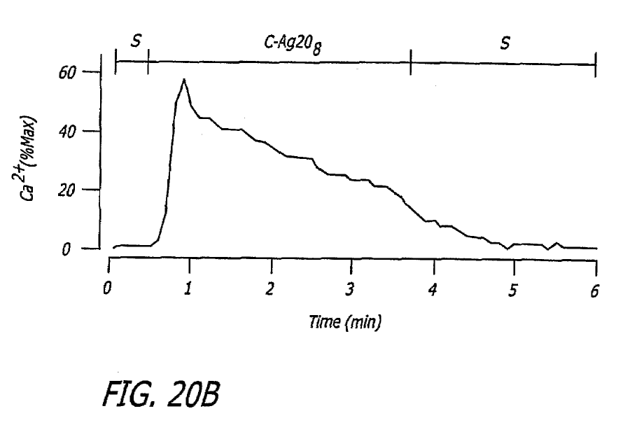

Previous studies have shown that amino acid residues in the first extracellular loop of the Na+/K+-ATPase are critically important for ouabain sensitivity: pumps carrying Gln108Arg and Asn119Asp mutations are insensitive to ouabain. Functional similarity between agrin and ouabain, together with the ability of C-Ag15 to act as a ouabain antagonist (Hilgenberg et al., 2006), raised the possibility that ouabain and agrin bind to a common site. To test this hypothesis, the response of non-neuronal cerebro-cortical cells transfected with an α3Na+/K+-ATPase Gln108Arg/Asn119Asp expression construct (Duran et al., 2004) was examined. Na+ imaging with SBFI showed that treatment with C-Ag20 elicited a robust increase in cytoplasmic Na+ concentration in transfected but not non-transfected cells (FIG. 20). In light of these findings it can be shown that while agrin binding overlaps or occludes a domain required for high affinity binding of ouabain, other sites also play a role in agrin interaction with the α3Na+/K+-ATPase.

In summary, the data provided indicates that the spontaneous contraction of agrin-deficient cardiac myocytes growing in cell culture is 2-fold higher than wild type, which is strong evidence that the agrin-a3NA+/K+-ATPASE pathway is important in cardiac muscle contraction (FIG. 17). Modulation of the contractile properties of cardiac myocytes by treatment with C-Ag20 or C-Ag15 demonstrates the therapeutic potential of these agrin derivatives (FIG. 15).

### Example 11c

### Hypertension

Plasma levels of endogenous cardiac glyosides are elevated in humans with hypertension and in animal models (Blaustein et al., 2006) and binding of cardiac glycosides to high affinity NA+/K+-ATPASEs has been shown to play a role in regulation of blood pressure in mice (Dostanic et al., 2005). Interestingly, although a1NA+/K+-ATPASE is the major isoform in kidney, a3NA+/K+-ATPASE is highly expressed in human kidney collecting duct epithelium (Barlet-Bas et al., 1993). Agrin is also expressed in kidney (Raats et al., 1998) suggesting agrin modulation of NA+/K+-ATPASE activity is important for kidney function.

### Example 11d

### Cataract

By age 75 as many as 70% of Americans have cataracts that significantly impair vision. Studies of mouse mutant that develop cataracts suggest that NA+/K+-ATPASE functions is critical for normal lens transparency (Delamere and Tamiya, 2004). Human lens epithelial cells express α2 and α3 NA+/K+-ATPASEs, suggesting drugs based on knowledge of agrin-NA+/K+-ATPASE interactions may prove useful in treatment and prevention of cataract.

### Example 11e

### Glaucoma

Primary glaucoma is leading cause of blindness affecting about 3 million people in the U.S. Blindness results from damage to the ocular nerve due to the pathological rise in intraocular pressure. Intraocular pressure is determined by balance between aqueous humour production by ciliary body and drainage from Canal of Schlemm. The primary energy source driving fluid production by ciliary body is the sodium/potassium gradient set up by activity of NA+/K+-ATPASEs in ciliary epithelium. a1, a2, and a3 NA+/K+-ATPASE are expressed in the ciliary body (Ghosh et al., 1990; Ghosh et al., 1991) and the cardiac glycoside digoxin has been shown to induce a marked decrease in intraocular pressure (Ferraiolo and Pace, 1979), suggesting another area in which agrin and/or its derivatives might prove useful therapeutic agents.

### Example 11f

### Cancer

Changes in NA+/K+-ATPASE expression and/or function have been observed in many cancers and may therefore be linked to tumorigenesis. Clinical and epidemiological evidence suggest that ouabain and related cardiac glycosides are potentially important anti-cancer drugs. For example, patients with cardiac diseases treated with digitalis preparations suffer from less cancer and have a lower recurrence of pre-existing cancers than other patients. Some data to suggest that drugs influencing NA+/K+-ATPASE function may be particularly useful in breast cancer. Two mechanisms of action have been suggested: modulation of NA+/K+-ATPASE activity impacts tight junction and adhesive interactions that control cell growth and metastasis; ouabain and other cardiac glycosides may act as antagonists for membrane estrogen receptors and may therefore inhibit growth of tumors like breast cancers that are stimulated through this pathway (reviewed in Chen et al., 2005). Potential tie-in with agrin is possibility that misregulation of agrin might influence tumor development directly. Agrin derivatives may be useful inhibitor s of tumor growth.

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. In particular, it is to be understood that this invention is not limited to the particular methodology, protocols, cell lines, animal species or genera, constructs, and reagents described as such may vary, as will be appreciated by one of skill in the art. The scope of the invention is, therefore, indicated by the following claims rather than by the foregoing description. All changes, modifications, and variations coming within the meaning and range of equivalency of the claims are to be considered within their scope.

What is claimed is:

1. A method for screening therapeutic agents useful for treating disorders related to the function of the Na⁺/K^{+ pump} without neurological side effects, comprising the steps of:
contacting a potential therapeutic agent to an α3Na⁺/K⁺-ATPase receptor; and
evaluating the ability of the potential therapeutic agent to regulate α3Na⁺/K⁺-ATPase activity.

2. The method of claim 1, wherein the disorder is seizures, and the potential therapeutic agent is evaluated on its ability to potentiate α3Na⁺/K⁺-ATPase activity.

3. The method of claim 1, wherein the disorder is congestive heart failure, the potential therapeutic agent is a cardiac glycoside or other small molecule evaluated on its ability to inhibit α3Na⁺/K⁺-ATPase activity, and the method further comprised the steps of:
evaluating the ability of the potential therapeutic agent to inhibit α1Na⁺/K⁺-ATPase and/or α2Na⁺/K⁺-ATPase activity;
identifying those potential therapeutic agents that inhibit α1Na⁺/K⁺-ATPase and/or α2Na⁺/K⁺-ATPase activity but not α3Na⁺/K⁺-ATPase activities.

4. The method of claim 1, wherein the disorder is hypertension, the potential therapeutic agent is evaluated on its ability to inhibit α3Na⁺/K⁺-ATPase activity, and the method further comprised the steps of:
evaluating the ability of the potential therapeutic agent to inhibit α1Na⁺/K⁺-ATPase and/or α2Na⁺/K⁺-ATPase activity;
identifying those potential therapeutic agents that inhibit α1Na⁺/K⁺-ATPase and/or α2Na⁺/K⁺-ATPase activity but not α3Na⁺/K⁺-ATPase activities.

5. The method of claim 1, wherein the disorder is of the central nervous system.

6. The method of claim 5, wherein the disorder is excitotoxic injury, and the potential therapeutic agent is evaluated on its ability to inhibit α3Na⁺/K⁺-ATPase activity.

7. The method of claim 1, wherein the disorder is tumorigenesis.

8. The method of claim 1, wherein the disorder is cataract, and the method further comprised the steps of:
contacting a potential therapeutic agent to an α2Na⁺/K⁺-ATPase receptor; and
evaluating the ability of the potential therapeutic agent to regulate α2Na⁺/K⁺-ATPase activity.

9. The method of claim 1, wherein the disorder is glaucoma, and the method further comprised the steps of:
contacting a potential therapeutic agent to an α1Na⁺/K⁺-ATPase receptor and an α2Na^{÷}/K⁺-ATPase receptor; and
evaluating the ability of the potential therapeutic agent to regulate α1Na⁺/K⁺-ATPase receptor and α2Na⁺/K⁺-ATPase activity.

10. A method of screening biologically active agents that facilitate improvement in motor performance, the method comprising:
providing an ATP1a3^{loxP}/^{loxP} Th^{cre}/^{cre} transgenic mouse in which expression of Cre recombinase results in loss of α3Na⁺/K⁺-ATPase function in dopamine neurons, resulting in abnormal motor performance;
administering a candidate agent to the transgenic mouse; and
determining the effect of said agent upon motor performance.

11. An ATP1a3^{loxP}/^{loxP} Th^{cre}/^{cre} transgenic mouse in which expression of Cre recombinase results in loss of α3Na⁺/K⁺-ATPase function in dopamine neurons, resulting in abnormal motor performance.

12. A method for treating a disorder arising from an Na⁺/K⁺-ion pump dysregulation in an individual in need thereof, comprising administering to the individual a therapeutically effective amount of a polypeptide comprising an agrin fragment capable of regulating α3Na⁺/K⁺-ATPase activity.

13. The method of claim 12, wherein the disorder arises from an Na⁺/K⁺-ion pump deficiency, and comprising administering to the individual a therapeutically effective amount of a polypeptide comprising an approximately 20-kD C-terminal agrin fragment.

14. The method of claim 13, wherein the polypeptide has the amino acid sequence identified as SEQ. ID NO. 1.

15. The method of claim 13, wherein the polypeptide has the amino acid sequence identified as SEQ. ID NO. 2.

16. The method of claim 13, wherein the polypeptide is a homolog of the polypeptide having the amino acid sequence identified as SEQ. ID NO. 1.

17. The method of claim 13, wherein the polypeptide is a derivative of the polypeptide having the amino acid sequence identified as SEQ. ID NO. 1.

18. The method of claim 13, wherein the polypeptide is a homolog of the polypeptide having the amino acid sequence identified as SEQ. ID NO. 2.

19. The method of claim 13, wherein the polypeptide is a derivative of the polypeptide having the amino acid sequence identified as SEQ. ID NO. 2.

20. The method of claim 13, wherein the polypeptide is a peptidomimetic of the polypeptide having the amino acid sequence identified as SEQ. ID NO. 1.

21. The method of claim 13, wherein the polypeptide is a peptidomimetic of the polypeptide having the amino acid sequence identified as SEQ. ID NO. 2.

22. The method of claim 13, wherein the individual is a human.

23. The method of claim 13 wherein the disorder is congestive heart failure.

24. The method of claim 13 wherein the disorder is hypertension.

25. The method of claim 1, wherein the disorder arises from an Na⁺/K⁺-ion pump overexpression, and comprising administering to the individual a therapeutically effective amount of a polypeptide comprising an approximately 15-kD C-terminal agrin fragment.

26. The method of claim 25, wherein the polypeptide has the amino acid sequence identified as SEQ. ID NO. 3.

27. The method of claim 25, wherein the polypeptide is a homolog of the polypeptide having the amino acid sequence identified as SEQ. ID NO. 3.

28. The method of claim 25, wherein the polypeptide is a derivative of the polypeptide having the amino acid sequence identified as SEQ. ID NO. 3.

29. The method of claim 25, wherein the polypeptide is a peptidomimetic of the polypeptide having the amino acid sequence identified as SEQ. ID NO. 3.

30. The method of claim 25, wherein the individual is a human.

31. The method of claim 25, wherein the disorder is an excitotoxic injury to the central nervous system.

32. The method of claim 25, wherein the disorder is a disturbance of locomotor function.

33. The method of claim 25, wherein the disorder is Parkinson's disease.

34. The method of claim 25 wherein the disorder is congestive heart failure.

35. The method of claim 25 wherein the disorder is hypertension.

36. A method of manufacturing a medicament for use in treating ion pump overexpression disorders in a mammal, the method comprising:
(a) providing a composition in dosage form, which comprises a synthetic polypeptide comprising an approximately 15-kD C-terminal agrin fragment;
(b) packaging the composition; and
(c) providing the package with a label instructing a user to administer the composition as a medicament for use in treating seizures in a mammal.

37. The method of claim 36, wherein the polypeptide has the amino acid sequence identified as SEQ. ID NO. 3.

38. The method of claim 36, wherein the polypeptide is a homolog of the polypeptide having the amino acid sequence identified as SEQ. ID NO. 3.

39. The method of claim 36, wherein the polypeptide is a derivative of the polypeptide having the amino acid sequence identified as SEQ. ID NO. 3.

40. The method of claim 36, wherein the polypeptide is a peptidomimetic of the polypeptide having the amino acid sequence identified as SEQ. ID NO. 3.

41. The method of claim 36, wherein the mammal is a human.

42. A method of manufacturing a medicament for use in treating an ion pump deficiency disorder in a mammal, the method comprising:
(a) providing a composition in dosage form, which comprises a synthetic polypeptide comprising an approximately 20-kD C-terminal agrin fragment;
(b) packaging the composition; and
(c) providing the package with a label instructing a user to administer the composition as a medicament for use in rescuing an agrin-deficient phenotype in a mammal.

43. The method of claim 42, wherein the polypeptide has the amino acid sequence identified as SEQ. ID NO. 1.

44. The method of claim 42, wherein the polypeptide has the amino acid sequence identified as SEQ. ID NO. 2.

45. The method of claim 42, wherein the polypeptide is a homolog of the polypeptide having the amino acid sequence identified as SEQ. ID NO. 1.

46. The method of claim 42, wherein the polypeptide is a derivative of the polypeptide having the amino acid sequence identified as SEQ. ID NO. 1.

47. The method of claim 42, wherein the polypeptide is a homolog of the polypeptide having the amino acid sequence identified as SEQ. ID NO. 2.

48. The method of claim 42, wherein the polypeptide is a derivative of the polypeptide having the amino acid sequence identified as SEQ. ID NO. 2.

49. The method of claim 42, wherein the polypeptide is a peptidomimetic of the polypeptide having the amino acid sequence identified as SEQ. ID NO. 1.

50. The method of claim 42, wherein the polypeptide is a peptidomimetic of the polypeptide having the amino acid sequence identified as SEQ. ID NO. 2.

51. The method of claim 42, wherein the mammal is a human.


SEQUENCE LISTING (9 sequences)

SEQ ID NO: 1 - protein, 204 aa, Artificial sequence
  Synthetically produced sequence
MDPRTYIEYLNAVTESEKALQSNHFELTLRTEATQGWLVLWIGKAAERADYMALAIVDGHLQLSYDLASQPVVLRSTVKVNTNRWLRVRAHREHREGSLQVGNEAPVTSSSPLGATQLDTDGALWLGGLQKLPVGQPLPKAYGTGFVGCLRNVVGHRQLHLLEDAVTKPELRPCPTLNSKLGPEQKLISEEDLNSAVDHHHHHH

SEQ ID NO: 2 - protein, 212 aa, Artificial sequence
  Synthetically produced sequence
MDPRTYIEYLNAVTESELTNEIPAEKALQSNHFELTLRTEATQGWLVLWIGKAAERADYMALAIVDGHLQLSYDLASQPVVLRSTVKVNTNRWLRVRAHREHREGSLQVGNEAPVTSSSPLGATQLDTDGALWLGGLQKLPVGQPLPKAYGTGFVGCLRNVVGHRQLHLLEDAVTKPELRPCPTLNSKLGPEQKLISEEDLNSAVDHHHHHH

SEQ ID NO: 3 - protein, 167 aa, Artificial sequence
  Synthetically produced sequence
MDPWIGKAAERADYMALAIVDGHLQLSYDLASQPVVLRSTVKVNTNRWLRVRAHREHREGSLQVGNEAPVTSSSPLGATQLDTDGALWLGGLQKLPVGQPLPKAYGTGFVGCLRNVVGHRQLHLLEDAVTKPELRPCPTLNSKLGPEQKLISEEDLNSAVDHHHHHH

SEQ ID NO: 4 - protein, 23 aa, Artificial sequence
  Synthetically produced sequence
AATGGATCCTCGGACCTACATCG

SEQ ID NO: 5 - protein, 23 aa, Artificial sequence
  Synthetically produced sequence
TTCGAATTCAGAGTGGGGCAGGG

SEQ ID NO: 6 - protein, 24 aa, Artificial sequence
  Synthetically produced sequence
AATGGATCCGTGGATTGGCAAGGC

SEQ ID NO: 7 - protein, 26 aa, Artificial sequence
  Syntheetically produced sequence
TAGGATCCACCGCCAGTATTGACCGA

SEQ ID NO: 8 - protein, 26 aa, Artificial sequence
  Synthetically produced sequence
TAGATATCAGAGTGGGGCAGGGTCTT

SEQ ID NO: 9 - protein, 26 aa, Artificial sequence
  Synthetically produced sequence
TAGATATCGTCCGCCCATCAAAGGCC